(19) United States
(12) Patent Application Publication
(10) Pub. No.: US 2008/0020148 A1
(43) Pub. Date: Jan. 24, 2008

(54) Chromane Derivatives Method for Production and the Use Thereof

(76) Inventors: Markus Klein, Weiterstadt (DE); Peer Kirsch, Kanagawa (JP); Eike Poetsch, Muehltal (DE); Michael Heckmeier, Hemsbach (DE); Peter Best, Darmstadt (DE); Andreas Taugerbeck, Darmstadt (DE); Melanie Klasen-Memmer, Heuchelheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/576,859

(22) PCT Filed: Sep. 28, 2005
(86) PCT No.: PCT/EP05/10458

§ 371 Date: Apr. 6, 2007

(30) Foreign Application Priority Data
Oct. 7, 2004 (DE) 10 2004 048853.3

(51) Int. Cl. C09K 19/34; C07C 33/38; C07C 47/00; C07D 305/02; C07D 311/04; C07D 311/58; C07D 311/60; C07D 407/00

(52) U.S. Cl. 428/1.1; 252/299.01; 549/370; 549/398; 549/510; 568/442; 568/807

(57) ABSTRACT

The present invention relates to chromane derivatives of the general formula I 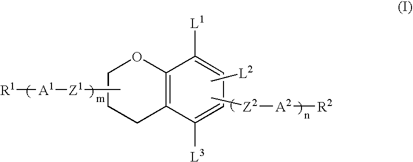
and of the general formula (II) 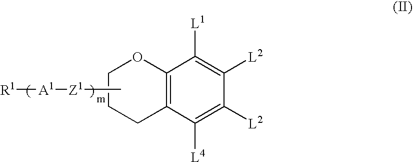
and chromene derivatives of the general formulae (III) and (IV) 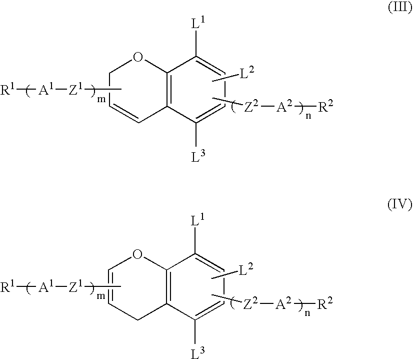
and chromene derivatives of the general formulae (V) and (VI) 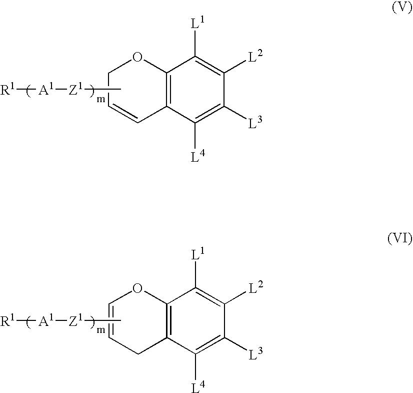
in which R¹, R², A¹, A², Z¹, Z², L¹, L², L³, L⁴, m and n have the meanings indicated in Claim 1 in relation to the respective formulae, to a process for the preparation thereof, to the use thereof as component(s) in liquid-crystalline media, and to liquid-crystal and electro-optical display elements which contain the liquid-crystalline media according to the invention.

The present invention relates to chromane derivatives, to a process for the preparation thereof, and to the use thereof as component(s) in liquid-crystalline media. In addition, the present invention relates to liquid-crystal and electro-optical display elements which contain the liquid-crystalline media according to the invention.

The liquid-crystalline compounds according to the invention can be used as component(s) of liquid-crystalline media, in particular for displays based on the principle of the twisted cell, the guest/host effect, the effect of deformation of aligned phases DAP or ECB (electrically controlled birefringence), the IPS (in-plane switching) effect or the effect of dynamic scattering.

Benzo-fused oxygen heterocyclic compounds are suitable components for liquid-crystalline mixtures which can be used in liquid-crystal and electro-optical display elements.

Thus, dihydrobenzofuran and chromane derivatives of the following formula 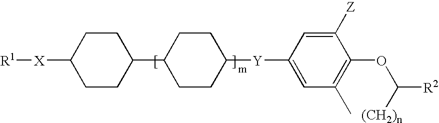
as components of liquid-crystalline mixtures are disclosed in JP 06/256337, where R¹, R², X, Y, Z, m and n have the meanings indicated in this document.

Chromane derivatives of the following formula 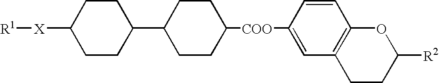
as components of liquid-crystalline mixtures are disclosed in JP 06/256339, where R¹, R² and X have the meanings indicated in this document.

In addition, the above-mentioned documents also disclose processes for the preparation of benzofuran and chromane derivatives.

The invention had the object of finding novel, stable, liquid-crystalline or mesogenic compounds which are suitable as component(s) of liquid-crystalline media, in particular for TN, STN, IPS, TFT and VA displays.

In addition, an object of the present invention was to provide liquid-crystalline compounds which have high dielectric anisotropy Δ∈, either positive or negative depending on the substitution. In addition, the compounds according to the invention should be thermally, chemically and photo-chemically stable. Furthermore, the compounds according to the invention should have the broadest possible nematic phase and be highly miscible with nematic base mixtures, in particular at low temperatures.

Surprisingly, it has been found that the chromane derivatives according to the invention are eminently suitable as component(s) of liquid-crystalline media. They can be used to obtain stable, liquid-crystalline media, suitable in particular for TFT or STN displays. The compounds according to the invention are both thermally and UV stable. They are also distinguished by high dielectric anisotropies Δ∈, owing to which lower threshold voltages are necessary on use. In addition, the compounds according to the invention have a broad nematic phase range and a high voltage holding ratio. Also advantageous is the good solubility of the compounds according to the invention, owing to which they are particularly suitable for increasing the low-temperature stability of polar liquid-crystal mixtures.

Through a suitable choice of the ring members and/or the terminal substituents, the physical properties of the liquid crystals according to the invention can be varied in broad ranges.

Since the chromane unit has a length between that of the conventional six-membered monocyclic and bicyclic rings, the derivatives according to the invention are in addition distinguished by positive elastic properties.

Liquid-crystalline media having very small values of the optical anisotropy are of particular importance for reflective and transflective applications, i.e. applications in which the respective LCD experiences no or only supporting backlighting.

The provision of the chromane derivatives according to the invention very generally considerably broadens the range of liquid-crystalline substances which are suitable, from various applicational points of view, for the preparation of liquid-crystalline mixtures.

The chromane derivatives according to the invention have a broad range of applications. Depending on the choice of substituents, these compounds can serve as base materials of which liquid-crystalline media are predominantly composed. However, it is also possible to add liquid-crystalline base materials from other classes of compound to the compounds according to the invention in order, for example, to modify the dielectric and/or optical anisotropy of a dielectric of this type and/or to optimise its threshold voltage and/or its viscosity.

In the pure state, the chromane derivatives according to the invention are colourless and form liquid-crystalline mesophases in a temperature range which is favourably located for electro-optical use. They are stable chemically, thermally and to light.

The present invention thus relates to chromane derivatives of the general formula (I) 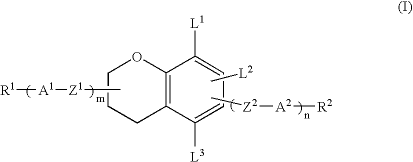
in which
R¹ denotes H, halogen (F, Cl, Br, I), or a linear or branched, optionally chiral alkyl radical having 1 to 15 C atoms or alkenyl radical having 2 to 15 C atoms which is unsubstituted, mono-substituted by CN or CF₃ or at least monosubstituted by halogen, in which, in addition, one or more CH₂ groups may each, independently of one another, be replaced by —O—, —S—, —CO—, —CO—O—, —O—CO—, —O—CO—O—, —CH═CH—, —CH═CF—, —CF═CH—, —CF═CF—, —C≡C— or [symbol] in such a way that hetero atoms are not linked directly to one another,
R² denotes H, F, Cl, NCS, CN, SF₅, an alkyl or alkoxy radical having 1 to 15 C atoms, an alkenyl or alkenyloxy radical having 2 to 15 C atoms, an alkyl or alkoxy radical having 1 to 15 C atoms which is substituted by one or more fluorine atoms, or an alkenyl or alkenyloxy radical having 2 to 15 C atoms which is substituted by one or more fluorine atoms,
A¹, A² each, independently of one another, identically or differently, denote
   a) trans-1,4-cyclohexylene, in which, in addition, one or more non-adjacent CH₂ groups may be replaced by —O— and/or —S—,
   b) 1,4-phenylene, in which one or two CH groups may be replaced by N and in which, in addition, one or more H atoms may be replaced by F,
   c) a radical from the group 1,4-bicyclo(2,2,2)octylene, piperidine-1,4-diyl, naphthalene-2,6-diyl, decahydronaphthalene-2,6-diyl and 1,2,3,4-tetrahydronaphthalene-2,6-diyl, or
   d) 1,4-cyclohexenylene,
Z¹, Z² each, independently of one another, identically or differently, denote —O—, —CH₂O—, —OCH₂—, —CO—O—, —O—CO—, —CF₂O—, —OCF₂—, —CF₂CF₂—, —CH₂CF₂—, —CF₂CH₂—, —CH₂CF₂O—, —OCF₂CH₂—, —CH₂CH₂—, —CH═CH—, —CH—CF—, —CF═CH—, —CF═CF—, —CF═CF—CO—O—, —O—CO—CF═CF—, —C≡C— or a single bond,
L¹, L², L³ each, independently of one another, identically or differently, denote H, F, CO, NCS, CN, SF₅, an alkyl or alkoxy radical having 1 to 15 C atoms which is substituted by one or more fluorine atoms, or an alkenyl or alkenyloxy radical having 2 to 15 C atoms which is substituted by one or more fluorine atoms, preferably H, F, Cl or CN, and particularly preferably H or F,
m denotes 0, 1, 2, 3 or 4, preferably 0, 1, 2 or 3 and particularly preferably 0, 1 or 2, and
n denotes 1, 2, 3 or 4, preferably 1, 2 or 3 and particularly preferably 1 or 2,
   but with the proviso that the sum (m+n)=1, 2, 3 or 4, preferably 1, 2 or 3 and particularly preferably 1 or 2,
   and of the general formula (II) 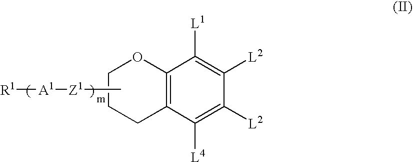
   in which R¹, A¹ and Z¹ have the meanings indicated in relation to the formula (I),
L¹, L², L³ and L⁴ each, independently of one another, identically or differently, denote H, F, Cl, NCS, CN, SF₅, an alkyl or alkoxy radical having 1 to 15 C atoms which is substituted by one or more fluorine atoms, or an alkenyl or alkenyloxy radical having 2 to 15 C atoms which is substituted by one or more fluorine atoms, preferably H, F, Cl or CN, and particularly preferably H or F, where
   one of the two radicals L² and L³ may additionally also adopt the meaning of R² in relation to the formula (I) and
   L² and L³ together may also denote 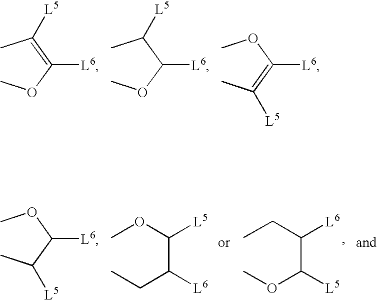
L¹ and L⁶ each, independently of one another, identically or differently, denote H, F, Cl or CN, and one of the two radicals additionally also denotes -(Z²-A²-)ₙR²,
   but with the proviso that, if L⁵ and L⁶ each, independently of one another, identically or differently, denote H, F, Cl or CN, m=1, 2, 3 or 4, preferably 1, 2 or 3 and particularly preferably 1 or 2, and that, if one of the two radicals denotes -(Z²-A²-)ₙR², m and n each, independently of one another, identically or differently, are 0, 1, 2, 3 or 4, where the sum (m+n) 1, 2, 3 or 4, preferably 1, 2 or 3,
   and chromene derivatives of the general formulae (III) and (IV) 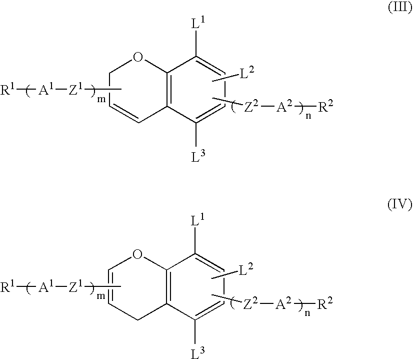
   in which R¹, R², A¹, A², Z¹, Z², L¹, L², L³ m and n have the meanings indicated in relation to the formula (I),
   and chromene derivatives of the general formulae (V) and (VI) 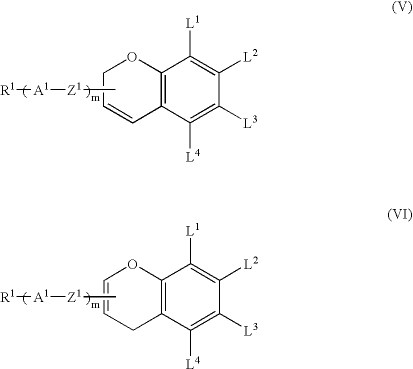
   in which R¹, A¹, Z¹, L¹, L², L³, L⁴ and m have the meanings indicated in relation to the formula (II).

Preference is given to the chromane derivatives of the general formulae (I) and (II).

The present invention furthermore relates to the use of chromane derivatives of the formulae (I) and (II) and chromene derivatives of the formulae (III) to (VI) as component(s) in liquid-crystalline media.

The present invention likewise relates to liquid-crystalline media having at least two liquid-crystalline components which comprise at least one chromane and/or chromene derivative of the formulae (I) to (VI).

The present invention also relates to liquid-crystal display elements, in particular electro-optical display elements, which contain, as dielectric, a liquid-crystalline medium according to the invention.

In a preferred embodiment, the compounds of the formulae (I) to (VI) according to the invention have a negative Δ∈. Owing to the negative Δ∈, these compounds are particularly suitable for use in VA displays.

The present invention thus also relates, in particular, to VA-TFT displays having dielectrics which comprise at least one chromane and/or chromene derivative of the formulae (I) to (VI) of negative Δ∈.

In a further preferred embodiment, the compounds of the formulae (I) to (VI) according to the invention have a positive Δ∈. Owing to the positive Δ∈, these compounds are particularly suitable for use in high-polarity mixtures.

The present invention thus also relates, in particular, to TFT displays having a low threshold voltage (so-called “low Vₜₕ TFT displays”) and IPS displays (so-called “in-plane switching displays”) having dielectrics which comprise at least one chromane and/or chromene derivative of the formulae (I) to (VI) of positive Δ∈.

If the compounds of the formulae (I) to (VI) according to the invention additionally, besides a positive Δ∈, also have a low birefringence Δn, these compounds are particularly suitable for use in reflective and transflective liquid-crystal display elements and other liquid-crystal displays having low birefringence Δn, so-called “low Δn mode displays”, such as, for example, reflective and transflective TN displays.

The present invention thus also relates, in particular, to reflective and transflective TN displays having dielectrics which comprise at least one chromane and/or chromene derivative of the formulae (I) to (VI) of positive Δ∈.

In addition, the chromane and chromene derivatives of the formulae (I) to (VI) according to the invention of positive Δ∈ are used as polar high-temperature clearing agents in displays operated at a temperature at which the control media are in the isotropic phase or in an optically isotropic phase. Such displays are described, for example, in DE-A-102 17 273, DE-A-102 53 325, DE-A-102 53 606 and DE-A-103 13 979.

The meaning of the formulae (I) to (VI) encompasses all isotopes of the chemical elements bound in the compounds of the formulae (I) to (VI). In enantiomerically pure or enriched form, the compounds of the formulae (I) to (VI) are also suitable as chiral dopants and in general for achieving chiral mesophases.

Above and below, R¹, R², A¹, A², Z¹, Z², L¹, L², L³, L⁴, L⁵, L⁶, m and n have the meanings indicated, unless expressly stated otherwise. If the radicals A¹ and Z¹ as well as A² and Z² occur more than once, they may, independently of one another, adopt identical or different meanings.

For reasons of simplicity, Cyc below denotes a 1,4-cyclohexylene radical, Che denotes a 1,4-cyclohexenylene radical, Dio denotes a 1,3-dioxane-2,5-diyl radical, Thp denotes a tetrahydropyran-2,5-diyl radical, Dit denotes a 1,3-dithiane-2,5-diyl radical, Phe denotes a 1,4-phenylene radical, Pyd denotes a pyridine-2,5-diyl radical, Pyr denotes a pyrimidine-2,5-diyl radical, Bco denotes a bicyclo(2,2,2)octylene radical and Dec denotes a decahydronaphthalene radical, where Cyc and/or Phe may be unsubstituted or mono- or polysubstituted by —CH₃, —Cl, —F and/or —CN.

Preference is given to compounds of the formulae (I) to (VI) in which R¹ denotes H, a linear alkyl or alkoxy radical having 1 to 10 C atoms or a linear alkenyl or alkenyloxy radical having 2 to 10 C atoms.

If R¹ is halogen, it preferably denotes F or Cl, particularly preferably F.

Preference is given to compounds of the formulae (I) to (VI) in which R² denotes F, Cl, ON, SF₅, CF₃, OCF₃ or OCHF₂, particularly preferably F, CN, CF₃ or OCF₃ and in particular F.

A¹ and A² preferably denote Phe, Cyc, Che, Pyd, Pyr or Dio and particularly preferably Phe or Cyc. Preference is furthermore given to compounds of the formulae (I) to (VI) which contain not more than one of the radicals Dio, flit, Pyd, Pyr or Bco.

Phe is preferably

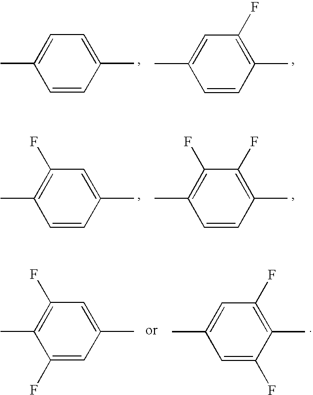

Phe is particularly preferably

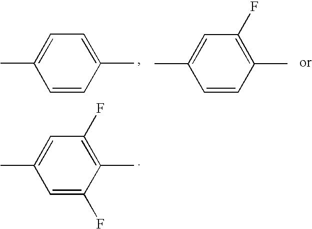

The terms 1,3-dioxane-2,5-diyl and Dio each encompass the two positional isomers

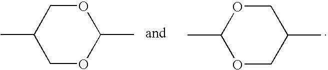

The cyclohexene-1,4-diyl group preferably has the following structures:

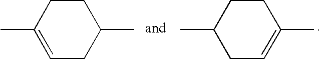

Z¹ and Z² preferably denote —CH₂CH₂—, —CH═CH—, —C≡C—, —CF₂CF₂—, —CF═CF—, —COO—, —OCO—, —CF₂O—, —OCF₂— or a single bond, particularly preferably —CF₂O—, —COO— or a single bond.

L¹, L², L³, L⁴, L⁵ and L⁶ preferably denote H or F.

Preferred chromane derivatives of the general formula (I) are represented by the following formulae (Ia) to (Id): 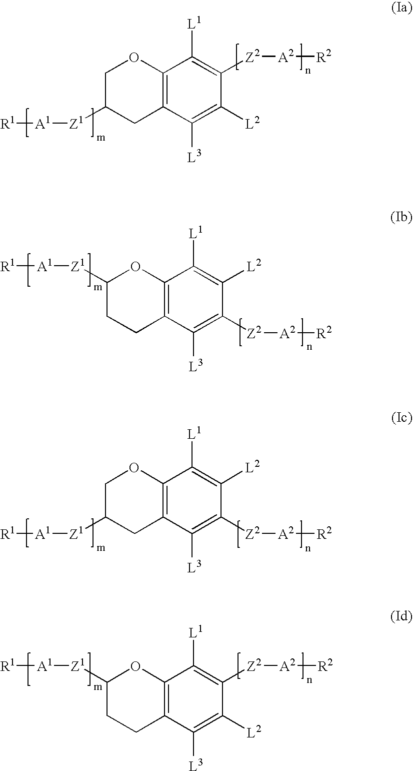
in which R¹, R², A¹, A², Z¹, Z², L¹, L², L³, m and n have the meanings indicated in relation to the formula (I).

Particular preference is given here to the chromane derivatives of the general formulae (Ia) and (Ib).

Preferred chromane derivatives of the general formula (Ia) are represented by the following formulae (Ia1) to (Ia6): 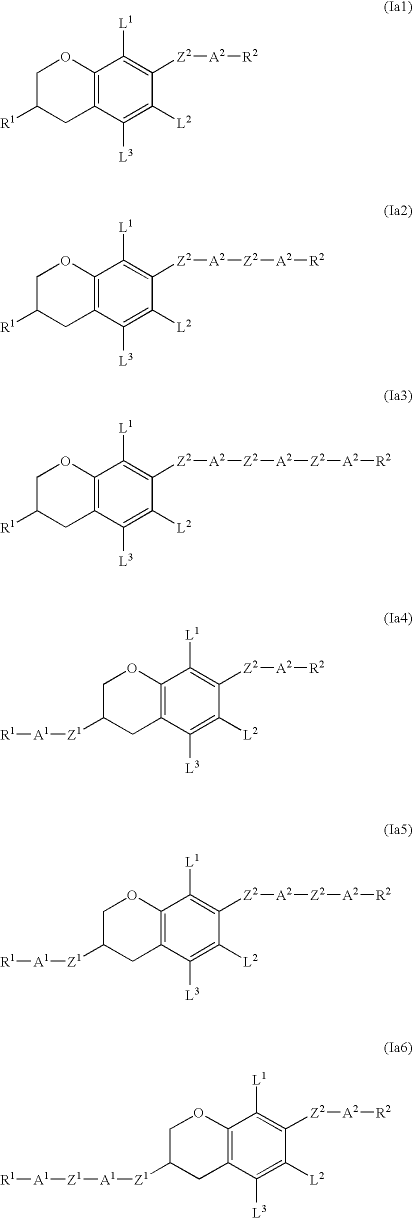
in which R¹, R², A¹, A², Z¹, Z², L¹, L² and L³ have the meanings indicated in relation to the formula (I).

Particular preference is given here to the chromane derivatives of the general formulae (Ia1) to (Ia5), i.e. chromane derivatives of the general formula (Ia) in which m=0 or 1.

Preference is furthermore given to chromane derivatives of the general formulae (Ia1) to (Ia6) in which L³=H and L¹ and L², independently of one another, identically or differently, denote H or F, it being particularly preferred if L¹=L²=F, L=H and L²=F or L¹=L²=H.

A particularly preferred compound of the sub-formula (Ia1) is that of the sub-formula (Ia1a): 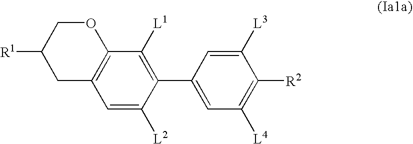
in which R¹ and R² have the meanings indicated in relation to the formula (I), and L¹, L², L³ and L⁴, independently of one another, identically or differently, denote H or F.

Particularly preferred compounds of the sub-formula (Ia2) are those of the sub-formulae (Ia2a) to (Ia2c): 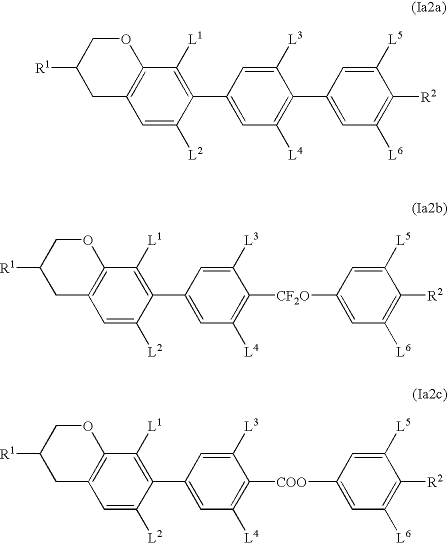
in which R¹ and R² have the meanings indicated in relation to the formula (I) and L¹, L², L³, L⁴, L⁵ and L⁶, independently of one another, identically or differently, denote H or F.

A particularly preferred compound of the sub-formula (Ia3) is that of the sub-formula (Ia3a): 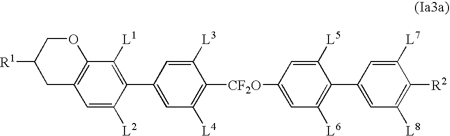
in which R¹ and R² have the meanings indicated in relation to the formula (I) and L¹, L², L³, L⁴, L⁵, L⁶, L⁷ and L⁸, independently of one another, identically or differently, denote H or F.

Particularly preferred compounds of the sub-formula (Ia4) are those of the sub-formulae (Ia4a) to (Ia4c): 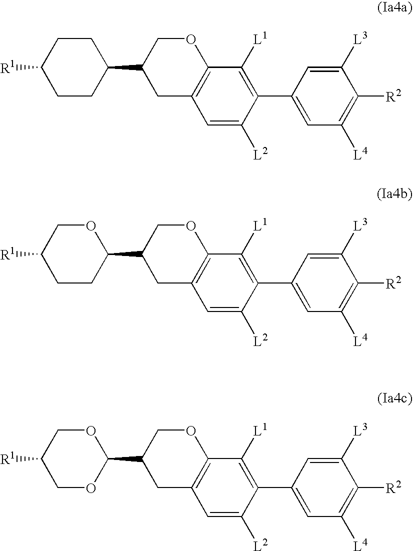
in which R¹ and R² have the meanings indicated in relation to the formula (I) and L¹, L², L³ and L⁴, independently of one another, identically or differently, denote H or F.

Particularly preferred compounds of the sub-formula (Ia5) are those of the sub-formulae (Ia5a) to (Ia5i), in particular those of the sub-formulae (Ia5a) to (Ia5c): 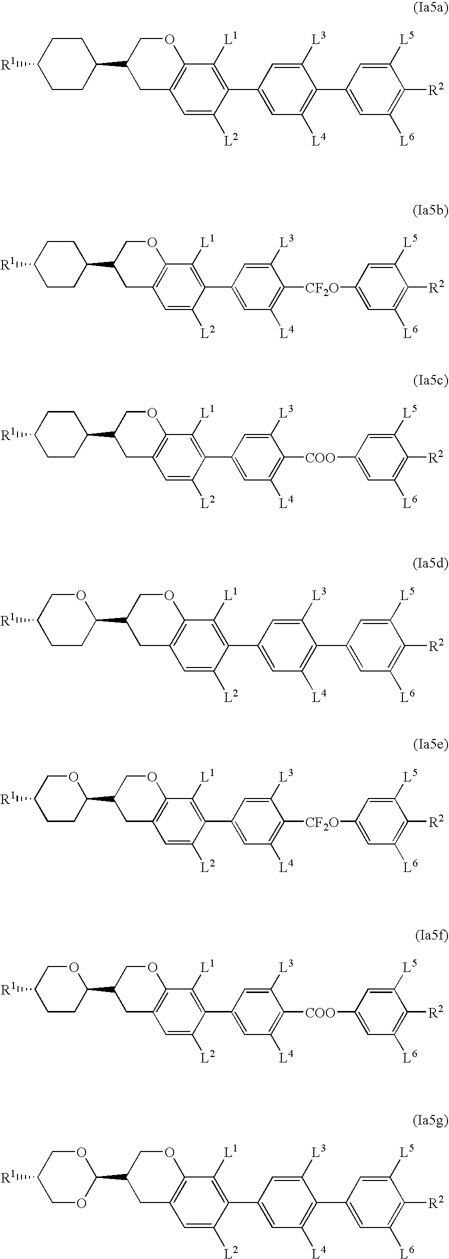 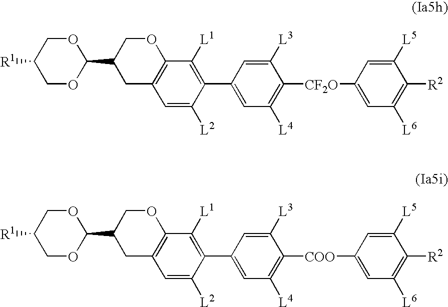
in which R¹ and R² have the meanings indicated in relation to the formula (I) and L¹, L², L³, L⁴, L⁵ and L⁶, independently of one another, identically or differently, denote H or F.

Preferred chromane derivatives of the general formula (Ib) are the following formulae (Ib1) to (Ib6): 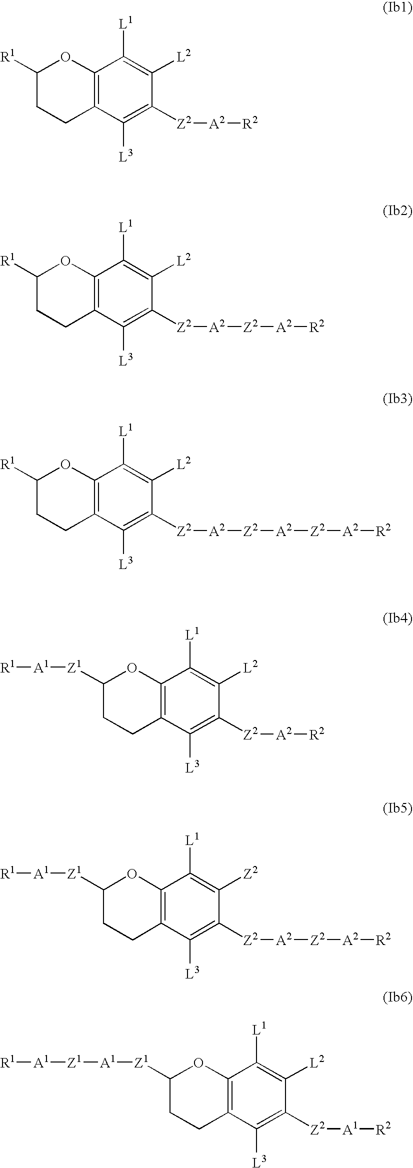
in which R¹, R², A¹, A², Z¹, Z², L¹, L² and L³ have the meanings indicated in relation to the formula (I).

Particular preference is given here to the chromane derivatives of the general formulae (Ib1), (Ib2) and (Ib4), i.e. chromane derivatives of the general formula (Ib) in which m=0 or 1 and the sum (m+n) is 1 or 2.

Preference is furthermore given to chromane derivatives of the general formulae (Ib1) to (Ib6) in which L³=H and L¹ and L², independently of one another, identically or differently, denote H or F, it being particularly preferred if at least one of the radicals L¹ and L² denotes F. In particular, L¹=L²=F.

Particularly preferred compounds of the sub-formula (Ib1) are those of the sub-formulae (Ib1a) to (Ib1c): 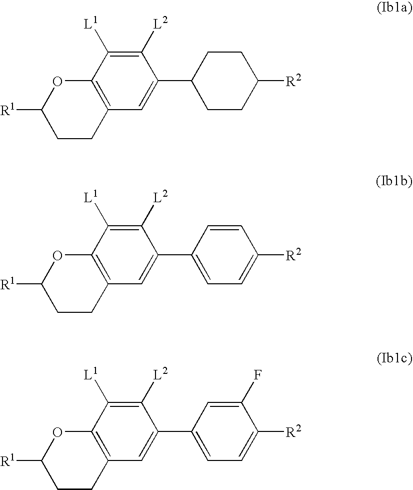
in which R¹ and R² have the meanings indicated in relation to the formula (I), and L¹ and L²; independently of one another, identically or differently, denote H or F, it being particularly preferred for at least one of the radicals L¹ and L² to denote F, but in particular both of the radicals.

Particularly preferred compounds of the sub-formula (Ib2) are those of the sub-formulae (Ib2a) to (Ib2c). 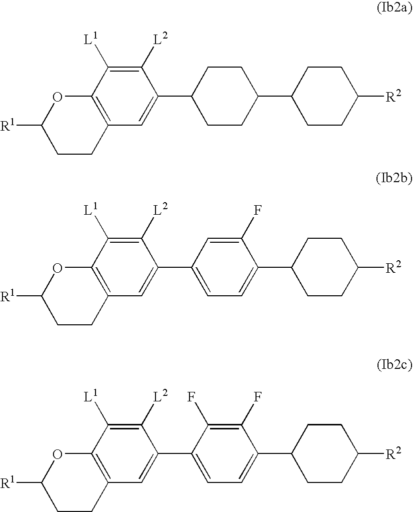
in which R¹ and R² have the meanings indicated in relation to the formula (I), and L¹ and L², independently of one another, identically or differently, denote H or F, it being particularly preferred for at least one of the radicals L¹ and L² to denote F, but in particular both of the radicals.

Particularly preferred compounds of the sub-formula (Ib4) are those of the sub-formulae (Ib4a) and (Ib4b): 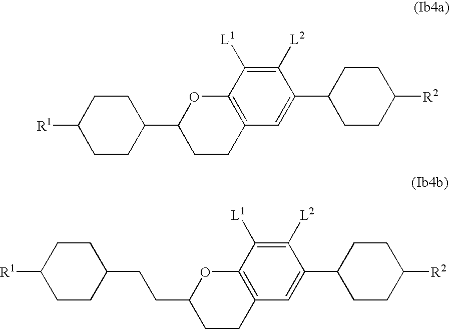
in which R¹ and R² have the meanings indicated in relation to the formula (I), and L¹ and L², independently of one another, identically or differently, denote H or F, it being particularly preferred for at least one of the radicals L¹ and L² to denote F, but in particular both of the radicals.

Preferred chromane derivatives of the general formula (II) are the following formulae (IIa) to (IId): 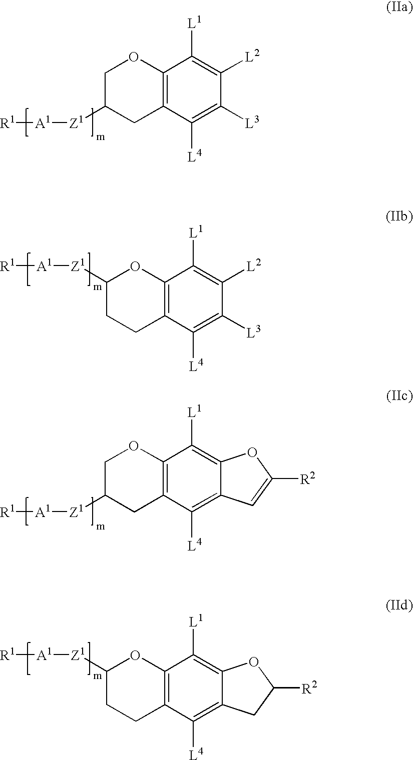
in which R¹, A¹, Z¹, L¹, L², L³ L⁴ and m have the meanings indicated in relation to the formula (II) and R² has the meanings indicated in relation to the formula (I).

Particular preference is given here to the chromane derivatives of the general formulae (IIa) and (IIb).

Preferred chromane derivatives of the general formula (IIa) are the following formulae (IIa1) to (IIa3): 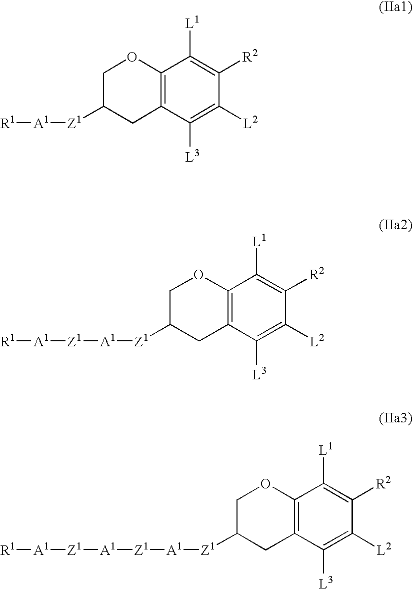
in which R¹, A¹, Z¹, L¹, L² and L³ have the meanings indicated in relation to the formula (II) and R² has the meanings indicated in relation to the formula (I).

Particularly preferred compounds of the sub-formula (IIa1) are those of the sub-formulae (IIa1a) and (IIa1b): 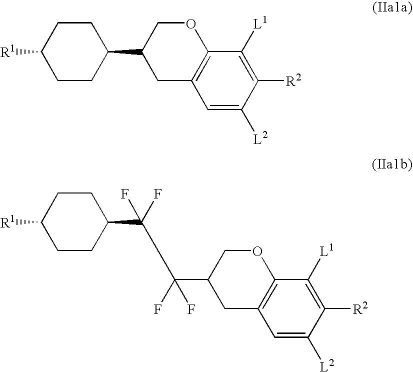
in which R¹ and R² have the meanings indicated in relation to the formula (I) and L¹ and L², independently of one another, identically or differently, denote H or F.

Preferred chromane derivatives of the general formula (IIb) are the following formulae (IIb1) to (IIb3): 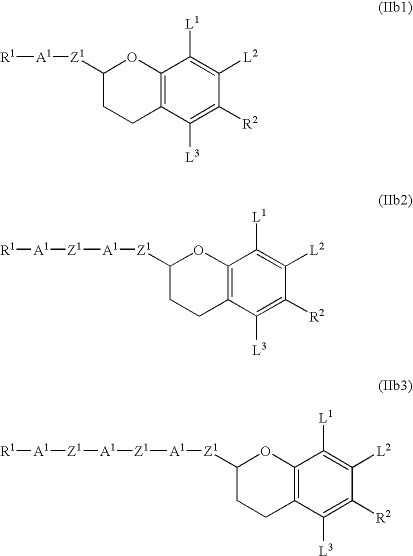
in which R¹, A¹, Z¹, L¹, L² and L³ have the meanings indicated in relation to the formula (II) and R² has the meanings indicated in relation to the formula (I).

Particularly preferred compounds of the sub-formula (IIb1) are those of the sub-formulae (IIb1a) and (IIb1b): 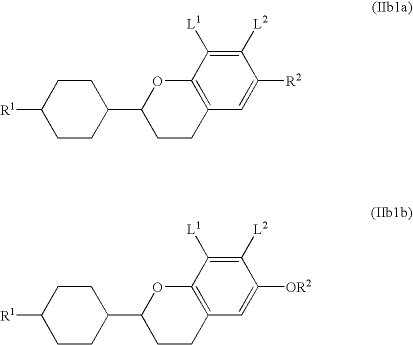
in which R¹ and R² have the meanings indicated in relation to the formula (I) and L¹ and L², independently of one another, identically or differently, denote H or F, it being particularly preferred for at least one of the radicals L¹ and L² to denote F, but in particular both of the radicals.

The chromane derivatives of the general formulae (IIa1) to (IIa3) preferably have the following structures: 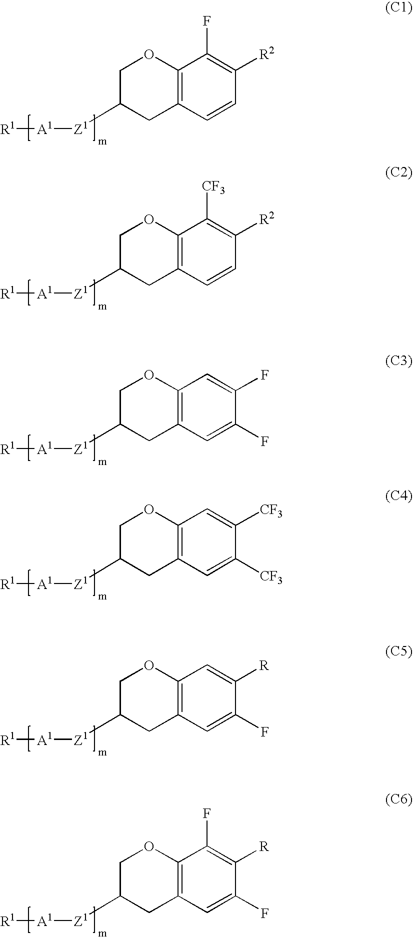
in which R¹, A¹ and Z¹ adopt the meanings indicated in relation to the formula (II) R² adopts the meanings indicated in relation to the formula (I), and m=1, 2 or 3.

The chromane derivatives of the general formulae (IIb1) to (IIb3) preferably have the following structures: 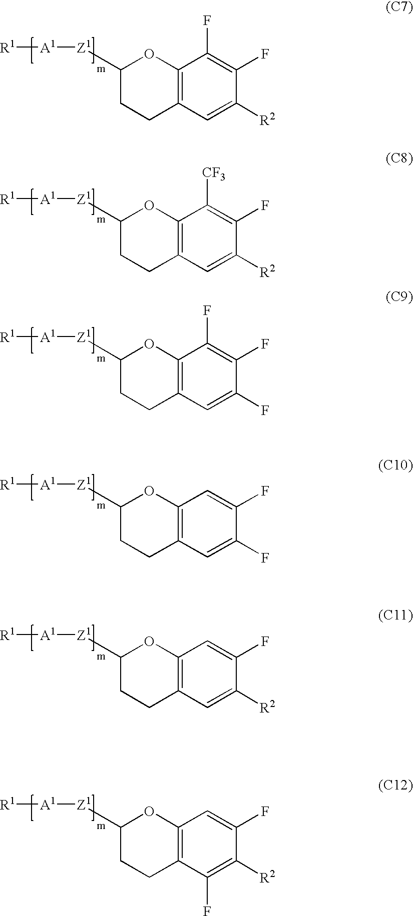
in which R¹, A¹ and Z¹ adopt the meanings indicated in relation to the formula (II), R² adopts the meanings indicated in relation to the formula (I), and m=1, 2 or 3.

A preferred chromane derivative of the general formula (IIc) is represented by the following formula (IIc1): 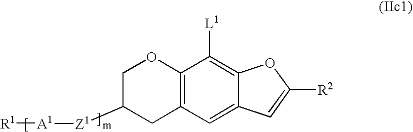
in which R¹, A¹, Z¹, m and L¹ have the meanings indicated in relation to the formula (II). L¹ preferably denotes F or CF₃, R² adopts the meanings indicated in relation to the formula (I).

A preferred chromane derivative of the general formula (IId) is the following formula (IId1): 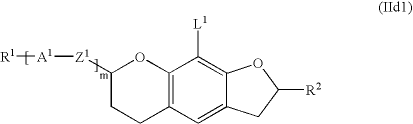
in which R¹, A¹, Z¹) m and L¹ have the meanings indicated in relation to the formula (II). L¹ preferably denotes F or CF₃. R² adopts the meanings indicated in relation to the formula (I).

The compounds of the formulae (II), (IIa) to (IId) and the sub-formulae thereof encompass compounds having one ring in the mesogenic group R¹(-A¹-Z¹)ₘ- of the sub-formulae a and b:
R¹-A a
R¹-A¹-Z¹- b
compounds having two rings in the mesogenic group R¹(-A¹-Z¹)ₘ- of the sub-formulae c to f:
R¹-A¹-A¹- c
R¹-A¹-A¹-Z¹- d
R¹-A¹-Z¹-A¹- e
R¹-A¹-Z¹-A¹-Z¹- f
and compounds having three rings in the mesogenic group R¹(-A¹-Z¹)ₘ- of the sub-formulae g to o:
R¹-A¹-A¹-A¹- g
R¹-A¹-Z¹-A¹-A¹- h
R¹-A¹-A¹-Z¹-A¹- i
R¹-A¹-A¹-A¹-Z¹- j
R¹-A¹-Z¹-A¹-Z¹-A¹- k
R¹-A¹-Z¹-A¹-A¹-Z¹- m
R¹-A¹-A¹-Z¹-A¹-Z¹- n
R¹-A¹-Z¹-A¹-Z¹-A¹-Z¹- o

Of these, particular preference is given to those of the sub-formulae a, b, c, d, e, g, h and i.

The preferred compounds of the sub-formula a encompass those of the sub-formulae aa to ad:
R¹-Phe- aa
R¹-Cyc- ab
R¹-Thp- ac
R¹-Dio- ad

Of these particular preference is given to those of the following sub-formulae:

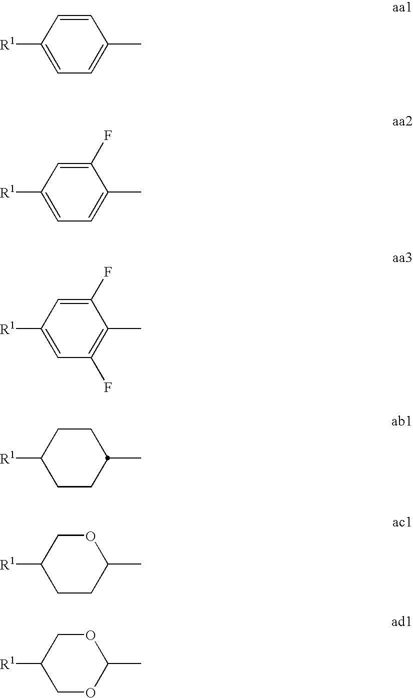

The preferred compounds of the sub-formula b encompass those of the sub-formulae ba and bb:
R¹-Phe-Z¹- ba
R¹-Cyc-Z¹- bb

The preferred compounds of the sub-formula Ic encompass those of the sub-formulae ca to cm:
R¹-Cyc-Cyc- ca
R¹-Cyc-Thp- cb
R¹-Cyc-Dio- cc
R¹-Cyc-Phe- cd
R¹-Thp-Cyc- ce
R¹-Dio-Cyc- cf
R¹-Phe-Cyc- cg
R¹-Thp-Phe- ch
R¹-Dio-Phe- ci
R¹-Phe-Phe- cj
R¹-Pyr-Phe- ck
R¹-Pyd-Phe- cm

Of these, particular preference is given to those of the following sub-formulae:

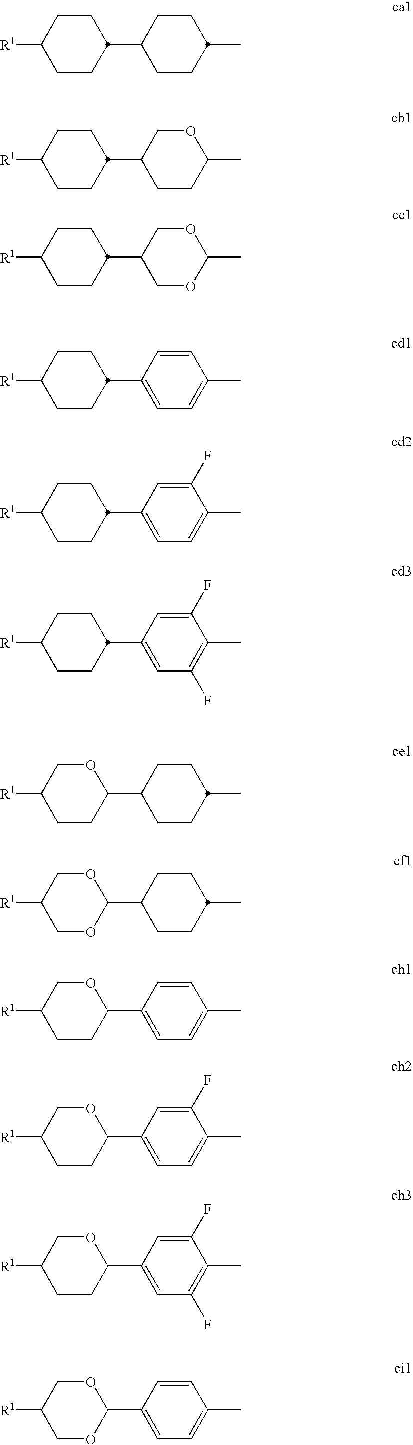

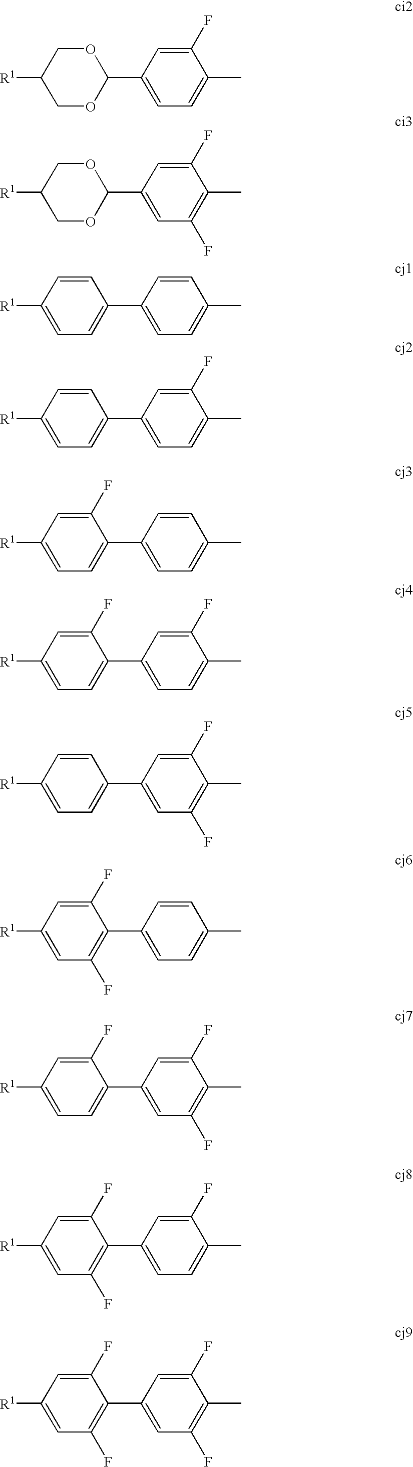

The preferred compounds of the sub-formula d encompass those of the sub-formulae da to dn:
R¹-Cyc-CYC-Z¹- da
R¹-Cyc-Thp-Z¹- db
R¹-Cyc-Dio-Z¹- dc
R¹-Cyc-Phe-Z¹- dd
R¹-Thp-Cyc-Z¹- de
R¹-Dio-Cyc-Z¹- df
R¹-Thp-Phe-Z¹- dg
R¹-Dio-Phe-Z¹- dh
R¹-Phe-Phe-Z¹- di
R¹-Pyr-Phe-Z¹- dj
R¹-Pyd-Phe-Z¹- dk
R¹-Cyc-Phe-CH₂CH₂— dm
R¹-A¹-Phe-CH₂CH₂— dn

Of these, particular preference is given to those of the following sub-formulae:

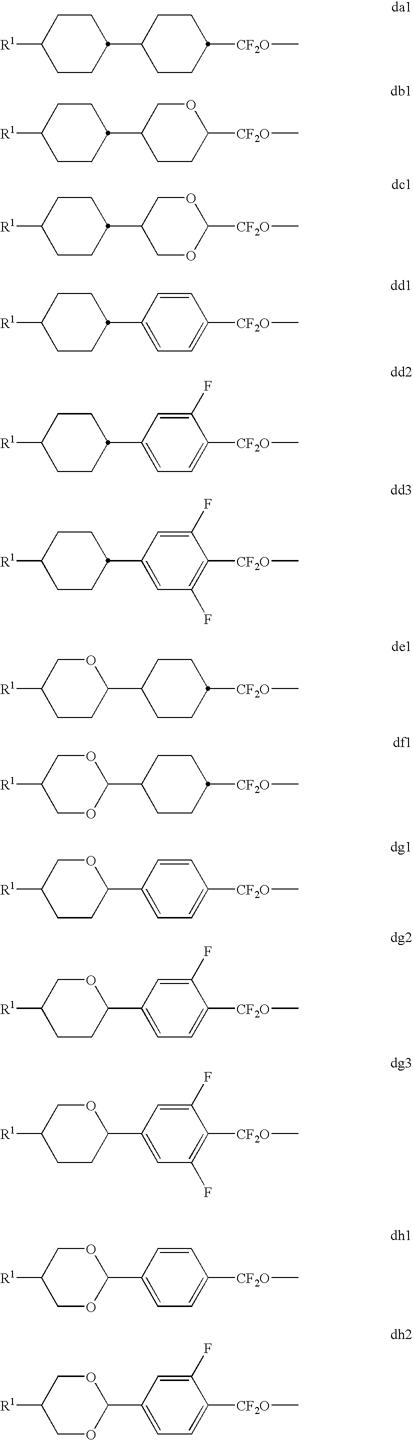

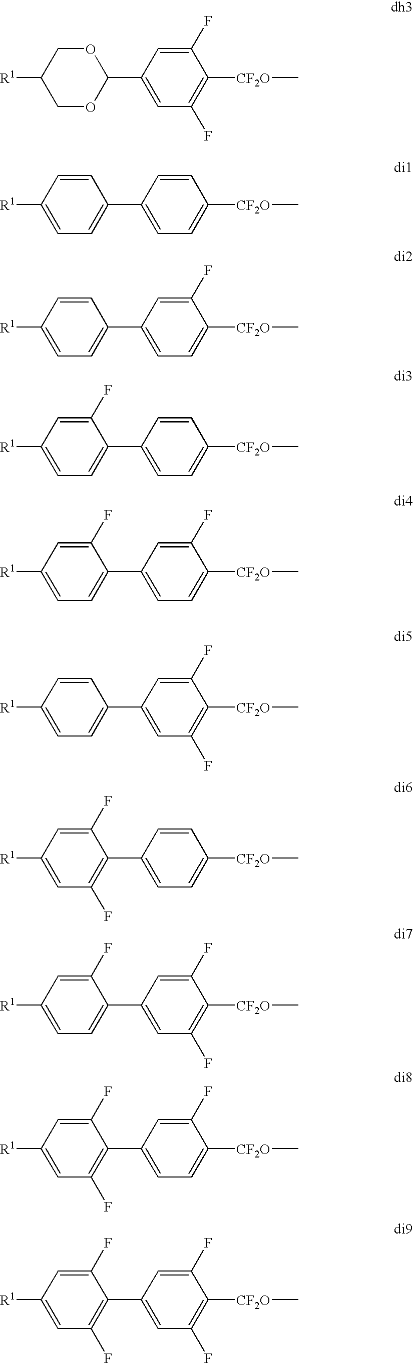

The preferred compounds of the sub-formula e encompass those of the sub-formulae ea to ej:
R¹-Cyc-Z¹-Cyc- ea
R¹-Thp-Z¹-Cyc- eb
R¹-A¹-CH₂CH₂-A¹- ec
R¹-Cyc-Z¹-Phe- ed
R¹-Thp-Z¹-Phe- ee
R¹-A¹-OCO-Phe- ef
R¹-Phe-Z¹-Phe- eg
R¹-Pyr-Z¹-A¹- eh
R¹-Pyd-Z¹-A¹- ei
R¹-Dio-Z¹-A¹- ej

Of these, particular preference is given to those of the following sub-formulae:

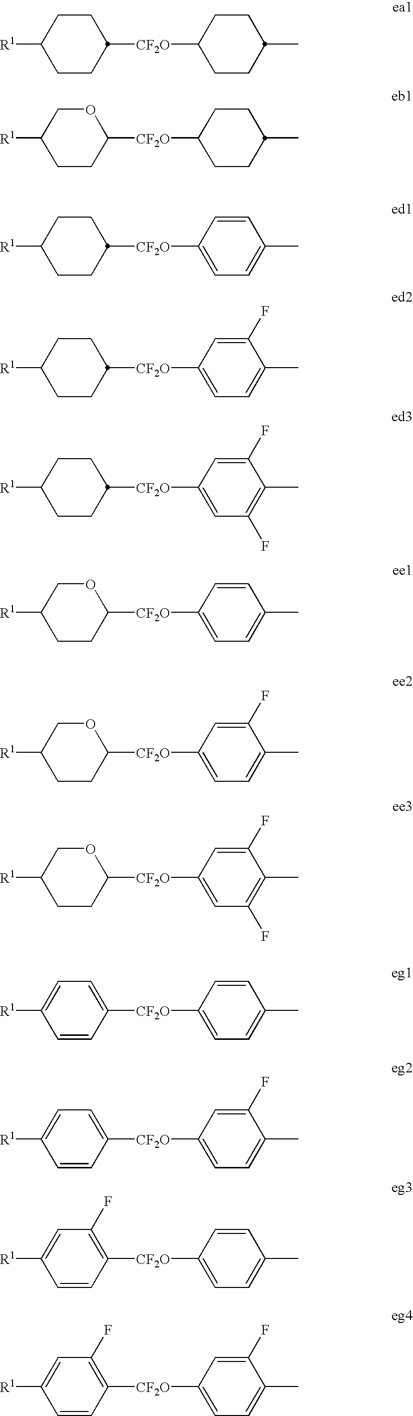

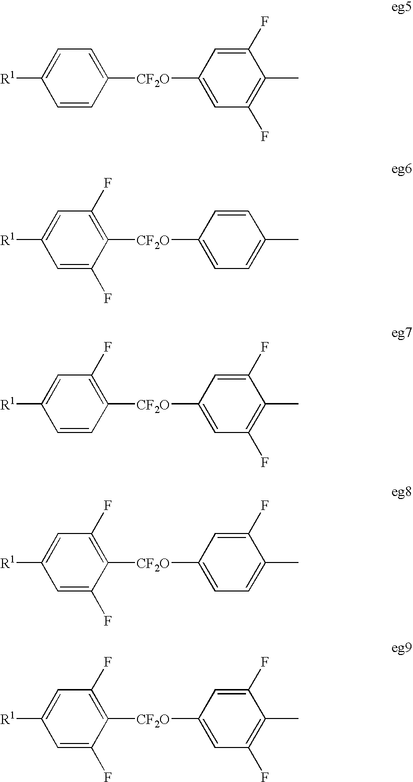

The preferred compounds of the sub-formula f encompass those of the sub-formulae fa to fe:
R¹-Phe-CH₂CH₂-A¹-Z¹- fa
R¹-A¹-COO-Phe-Z¹- fb
R¹-Cyc-Z¹-Cyc-Z¹- fc
R¹-Phe-Z¹-Phe-Z¹- fd
R¹-Cyc-CH₂CH₂-Phe-Z¹- fe

The preferred compounds of the sub-formulae g to n encompass those of the following sub-formulae ga to ma:
R¹-A¹-Cyc-Cyc- ga
R¹-A¹-Cyc-Phe- gb
R¹-Phe-Phe-Phe- gc
R¹-A¹-CH₂CH₂-A¹-Phe- ha
R¹-Phe-Z¹-A¹-Phe- hb
R¹-A¹-Phe-Z¹-Phe- ia
R¹-Cyc-Z¹-A¹-Z¹-Phe- ka
R¹-A¹-Z¹-Cyc-Phe-Z¹- ma

In the above preferred formulae, R¹, A¹ and Z¹ have the meanings indicated above. If A¹ and/or Z¹ occur more than once in one of the sub-formulae, they may, independently of one another, be identical or different.

In the above preferred formulae, A¹ preferably denotes a linear alkyl or alkoxy radical having 1 to 7 C atoms or a linear alkenyl or alkenyloxy radical having 2 to 7 C atoms and particularly preferably a linear alkyl radical having 1 to 7 C atoms or a linear alkenyl radical having 2 to 7 C atoms.

In the above preferred formulae, Z¹ preferably denotes —CH₂CH₂—, —C≡C—, —CF₂CF₂—, —COO—, —OCO—, —CF₂O— or —OCF₂—.

If R¹ or R² in the formulae above and below denotes an alkyl radical this may be straight-chain or branched. It is particularly preferably straight-chain, has 1, 2, 3, 4, 5, 6 or 7 C atoms and accordingly denotes methyl, ethyl, propyl, butyl, pentyl, hexyl or heptyl, furthermore octyl, nonyl, decyl, undecyl, dodecyl, tridecyl, tetradecyl or pentadecyl.

If R¹ or R² denotes an alkyl radical in which one CH₂ group has been replaced by —O—, this may be straight-chain or branched. It is preferably straight-chain and has 1 to 10 C atoms. The first CH₂ group in this alkyl radical has particularly preferably been replaced by —O—, so that the radical R¹ attains the meaning alkoxy and denotes methoxy, ethoxy, propoxy, butoxy, pentyloxy, hexyloxy, heptyloxy, octyloxy or nonyloxy.

Furthermore, a CH₂ group elsewhere may also have been replaced by —O—, so that the radical R¹ preferably denotes straight-chain 2-oxapropyl (=methoxymethyl), 2-(=ethoxymethyl) or 3-oxabutyl (=2-methoxyethyl), 2-, 3- or 4-oxapentyl, 2-, 3-, 4- or 5-oxahexyl, 2-, 3-, 4-, 5- or 6-oxaheptyl, 2-, 3-, 4-, 5-, 6- or 7-oxaoctyl, 2-, 3-, 4-, 5-, 6-, 7- or 8-oxanonyl, or 2-, 3-, 4-, 5-, 6-, 7-, 8- or 9-oxadecyl.

If R¹ or R² denotes an alkyl radical in which one CH₂ group has been replaced by —CH═CH—, this may be straight-chain or branched. It is preferably straight-chain and has 2 to 10 C atoms. Accordingly, it denotes vinyl, prop-1- or -2-enyl, but-1-, -2- or -3-enyl, pent-1-, -2-, -3- or -4-enyl, hex-1-, -2-, -3-, -4- or -5-enyl, hept-1-, -2-, -3-, -4-, -5- or -6-enyl, oct-1-, -2-, -3-, -4-, -5-, -6- or -7-enyl, non-1-, -2-, -3-, -4-, -5-, -6-, -7- or -8-enyl, or dec-1-, -2-, -3-, -4-, -5-, -6-, -7-, -8- or -9-enyl.

Preferred alkenyl groups are C₂-C₇-1E-alkenyl, C₄-C₇-3E-alkenyl, C₅-C₇-4-alkenyl, C₆-C₇-5-alkenyl and C₇-6-alkenyl, particularly preferably C₂-C₇-1E-alkenyl, C₄-C₇-3E-alkenyl and C₅-C₇-4-alkenyl.

Examples of particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl and 6-heptenyl. Groups having up to 5 carbon atoms are particularly preferred.

If R¹ denotes an alkyl radical in which one CH₂ group has been replaced by —O— and one has been replaced by —CO—, these are preferably adjacent. These thus contain an acyloxy group —CO—O— or an oxycarbonyl group —O—CO—. These are particularly preferably straight-chain and have 2 to 6 C atoms.

Accordingly, they denote in particular acetoxy, propionyloxy, butyryloxy, pentanoyloxy, hexanoyloxy, acetoxymethyl, propionyloxymethyl, butyryloxymethyl, pentanoyloxymethyl, 2-acetoxyethyl, 2-propionyloxyethyl, 2-butyryloxyethyl, 3-acetoxypropyl, 3-propionyloxypropyl, 4-acetoxybutyl, methoxycarbonyl, ethoxycarbonyl, propoxycarbonyl, butoxycarbonyl, pentoxycarbonyl, methoxycarbonylmethyl, ethoxycarbonylmethyl, propoxycarbonylmethyl, butoxycarbonylmethyl, 2-(methoxycarbonyl)ethyl, 2-(ethoxycarbonyl)ethyl, 2-(propoxycarbonyl)ethyl, 3-(methoxycarbonyl)propyl, 3-(ethoxycarbonyl)propyl or 4-(methoxycarbonyl)butyl.

If R¹ denotes an alkyl radical in which one CH₂ group has been replaced by unsubstituted or substituted —CH═CH— and an adjacent CH₂ group has been replaced by —CO—, CO—O— or —O—CO—, this may be straight-chain or branched. It is preferably straight-chain and has 4 to 13 C atoms. Accordingly, it particularly preferably denotes acryloyloxymethyl, 2-acryloyloxy-ethyl, 3-acryloyloxypropyl, 4-acryloyloxybutyl, 5-acryloyloxypentyl, 6-acryloyloxyhexyl, 7-acryloyloxyheptyl, 8-acryloyloxyoctyl, 9-acryloyloxynonyl, 10-acryloyloxydecyl, methacryloyloxymethyl, 2-methacryloyloxyethyl, 3-ethacryloyloxypropyl, 4-methacryloyloxybutyl, 5-methacryloyloxypentyl, 6-methacryloyloxyhexyl, 7-methacryloyloxyheptyl, 8-methacryloyloxyoctyl or 9-methacryloyloxynonyl.

If R¹ denotes an alkyl or alkenyl radical which is monosubstituted by CN or CF₃, this radical is preferably straight-chain and the substitution by CN or CF₃ is in the ω-position.

If R¹ or R² denotes an alkyl or alkenyl radical which is at least mono-substituted by halogen, this radical is preferably straight-chain. Halogen is preferably F or Cl. In the case of polysubstitution, halogen is preferably F. The resultant radicals also include perfluorinated radicals. In the case of monosubstitution, the fluorine or chlorine substituent may be in any desired position, but preferably in the ω-position.

Compounds of the formulae (I) to (VI) containing a branched wing group R¹ or R² may occasionally be of importance owing to better solubility in the conventional liquid-crystalline base materials, but in particular as chiral dopants if they are optically active. Smectic compounds of this type are suitable as component(s) of ferroelectric materials.

Branched groups of this type preferably contain not more than one chain branch. Preferred branched radicals R¹ or R² are isopropyl, 2-butyl (=1-methylpropyl), isobutyl (=2-methylpropyl), 2-methylbutyl, isopentyl (=3-methylbutyl), 2-methylpentyl, 3-methylpentyl, 2-ethylhexyl, 2-propylpentyl, isopropoxy, 2-methylpropoxy, 2-methylbutoxy, 3-methylbutoxy, 2-methylpentyloxy, 3-methylpentyloxy, 2-ethylhexyloxy, 1-methylhexyloxy and 1-methylheptyloxy.

The formulae (I) to (VI) encompass the racemates of these compounds and also the optical antipodes, and mixtures thereof.

Of the compounds of the formulae (I) to (VI) and the sub-formulae, preference is given to those in which at least one of the radicals present therein has one of the preferred meanings indicated.

In the compounds of the formulae (I) to (VI), preference is given to those stereoisomers in which the rings Cyc and piperidine are trans-1,4-disubstituted. Those of the above-mentioned formulae which contain one or more groups Pyd, Pyr and/or Dio in each case encompass the two 2,5-positional isomers.

The compounds of the general formulae (I) to (VI) can be prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can be made here of variants known per se, which are not mentioned here in greater detail.

The starting materials for the above processes are either known or can be prepared analogously to known compounds. They can thus be obtained by generally accessible literature procedures or commercially.

The starting materials can also, if desired, be formed in situ by not isolating them from the reaction mixture, but instead immediately converting them further into the compounds of the general formulae (I) to (VI).

A preferred synthesis of the compounds of the general formulae (Ib) and (III) can be carried out by the processes described in the literature, for example in Houben Weyl, Methoden der organischen Chemie [Methods of Organic Chemistry], Georg Thieme Vertag, Stuttgart, New York, 4th Edn. 1993.

A preferred process is the preparation of compounds of the general formula (Ib) by ring-closure metathesis of the correspondingly substituted dienes 3, which are accessible as described by S. Chang, R. H. Grubbs, J. Org. Chem. 1998, 63, 864-866. The chromenes of the general formula (III) obtained in this way can be converted into the chromanes of the general formula (Ib) by catalytic hydrogenation, as shown in scheme 1.

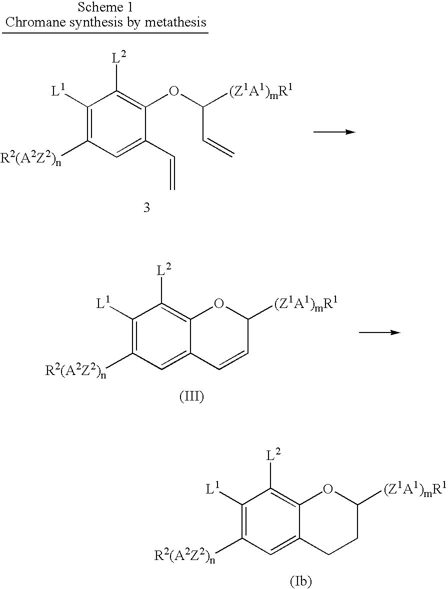

Alternatively, the compounds of the general formula (Ib) according to the invention can also be obtained by intramolecular cyclisation of diols, as described, for example, by S. Kelly, B. C. Vanderplas, in J. Org, Chem. 1991, 56, 1325-1327, and shown in Scheme 2.

Aldol condensation of the salicylaldehyde derivatives 4 with methyl ketones followed by hydrogenation and removal of the protecting group gives the ketones 5, which, after reduction to the alcohol 6, for example using sodium borohydride, cyclise to give the compounds of the formula (Ib) by subsequent treatment with sulfuric acid in glacial acetic acid.

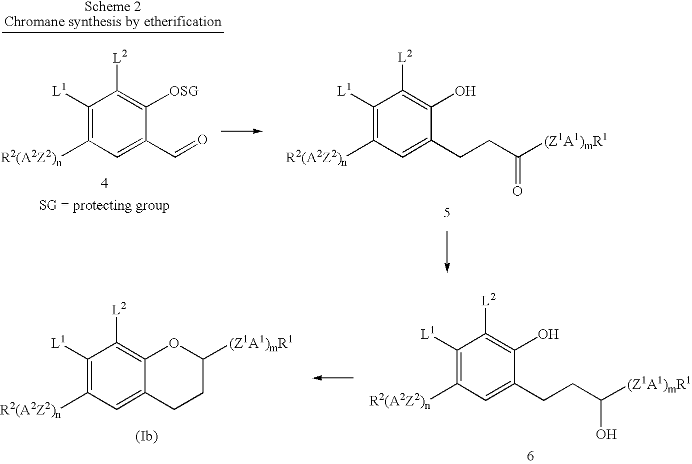

The starting material used for the compounds 3 and 4 can be salicylaldehydes. A possible process for the preparation of these salicylaldehydes is the reaction of commercial liquid-crystal precursors 7 in accordance with scheme 3 below.

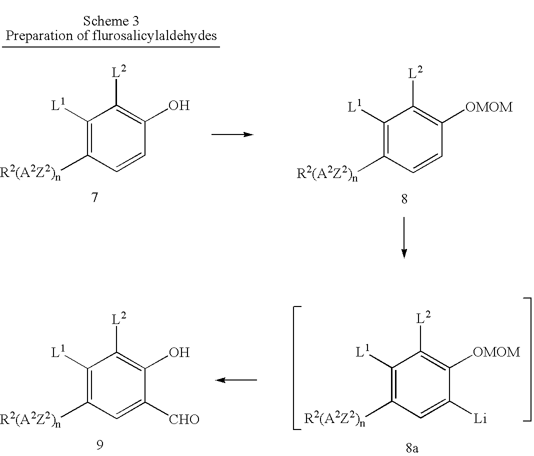

After conversion of the phenols 7 into a suitable derivative, for example MOM ether 8, the salicylaldehydes 9 can be obtained directly by ortho-metallation, scavenging using a formamide derivative, such as, for example, DMF, and subsequent deprotection, as described, for example, by I. R. Hardcastle, P. Quayle, E. L. M. Ward in Tetrahedron Lett. 1994, 35, 1747-1748.

Alternatively, the phenols 7 can also be firstly halogenated and subsequently, after protection of the hydroxyl group, metallated by halogen-lithium exchange and converted into salicylaldehydes analogously to scheme 4, as described, for example, by G. C. Finger, M. J. Gortakowski, R. H. Shiley, R. H. White in J. Amer. Chem. Soc. 1959, 81, 94-101 and shown in scheme 4.

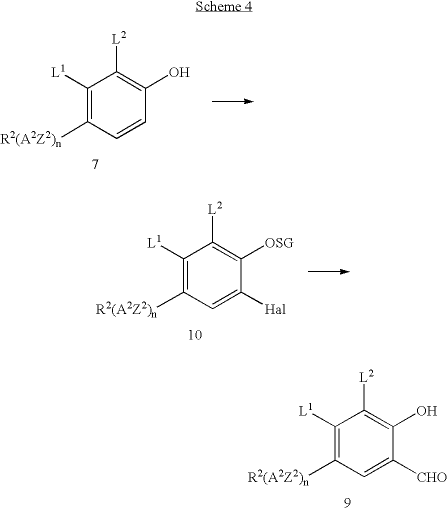

The chromane derivatives of the general formula (II) according to the invention are preferably prepared by
a) reacting an oxetane of the general formula (VIIa) or (VIIb) 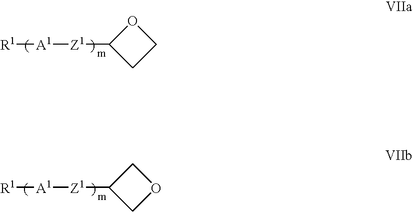
   in which R¹, A¹, Z¹ and m have the meanings indicated in relation to the formula (II) with an ortho-metallated fluoroaromatic compound in an organic solvent and at low temperatures to give the corresponding propanol derivative of the general formula (VIIIa) or (VIIIb) 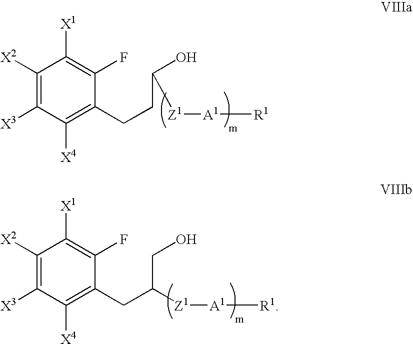
   in which R¹, A¹, X¹, X², X³, X⁴, Z¹ and m have the meanings indicated in relation to the formula (II), and
b) cyclising the resultant propanol derivative of the general formula (VIIIa) or (VIIIb) through the action of a strong, non-nucleophilic base to give the corresponding chromane derivative of the general formula II.

The chromane derivative obtained in this way can optionally be converted into the corresponding chromene derivative by dehydrogenation.

The reaction in step a) is preferably carried out in the presence of a Lewis acid. Lewis acids which can be employed here are in principle all compounds known to the person skilled in the art so long as they do not have acidic protons, Particular preference is given to strong Lewis acids, in particular BF₃ etherate. In the case of particularly reactive compounds, the reaction can also be carried out without the addition of a Lewis acid.

Organic solvents which can be employed in step a) are all solvents known for this purpose to the person skilled in the art. However, preferred solvents are diethyl ether, tetrahydrofuran (THF) and dimethoxyethane (DME), and mixtures thereof.

The term “low temperature” in the present application is taken to mean a temperature in the range from −40° C. to −100° C., preferably from −65° C. to −85° C.

The oxetanes can be prepared here by all processes known to the person skilled in the art. However, the starting materials are preferably diols of the following formulae, which are either commercially available or can be prepared easily. A process for their preparation is described, for example, in EP 0 967 261 B1. These diols can then be converted into oxetanes, for example by the process described by Picard et al., in: Synthesis, 1981, 550-552, as shown in scheme 5 below.

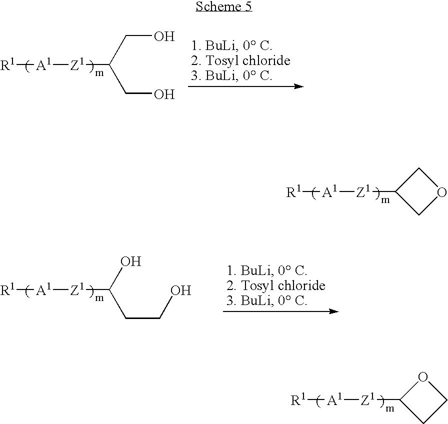

The ortho-metallated fluoroaromatic compounds can also be prepared by all processes known to the person skilled in the art. However, preferred processes are the ortho-metallation of fluoroaromatic compounds using butyllithium (BuLi), optionally with addition of TMEDA or similar compounds for increasing the reactivity of the aggregated butyllithium, Schlosser-Lochmann base or lithium diisopropylamide (LDA), in each case at low temperatures, or the halogen-metal exchange of iodofluoroaromatic compounds or bromofluoroaromatic compounds using BuLi at low temperatures (for example in accordance with Org. React. 6, 1951, 339-366) or using isopropylmagnesium chloride at temperatures in the range from −50° C. to −10° C. (Knochel et al., Angewandte Chemie, Int Ed. 42, 2003, 4302-4320).

If desired, this step can also be followed by a transmetallation. Thus, lithium aromatic compounds can easily be converted into the corresponding zinc aromatic compounds by reaction with a ZnCl₂ solution.

The ortho-metallated fluoroaromatic compound is then reacted with the oxetane in an organic solvent at low temperature, preferably in the presence of a Lewis acid, as shown in the two schemes 6a and 6b.

Depending on the oxetane used, the structurally isomeric alcohols can also be obtained in this way.

The oxetane is opened here with high regioselectivity on the less highly substituted side.

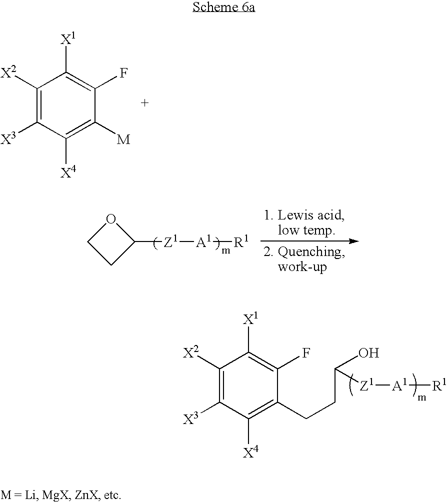

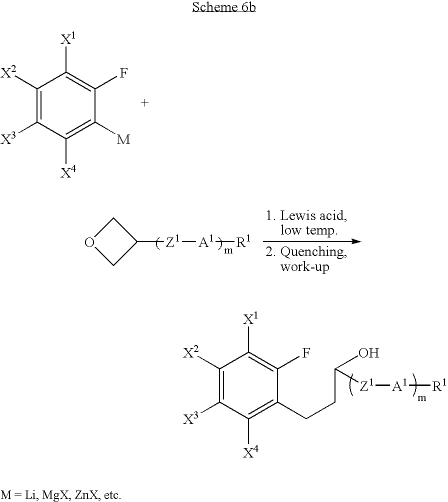

The propanol derivative formed from the ortho-metallated fluoroaromatic compound and the oxetane is subsequently subjected to intramolecular cyclisation in the presence of about 1 equivalent of a strong, non-nucleophilic base, for example alkali metal hydride, selected from NaH, KH, RbH or CsH, and potassium hexamethyldisilazane (KHMDS), preferably alkali metal hydride, particularly preferably KH, in an organic solvent. The reaction is shown in scheme 7 below. This cyclisation is preferably carried out at a temperature in the range between 0° C. and 78° C. Particular preference is given to the use of from 1 to 1.5 equivalents of potassium hydride (KH) in tetrahydrofuran (THF).

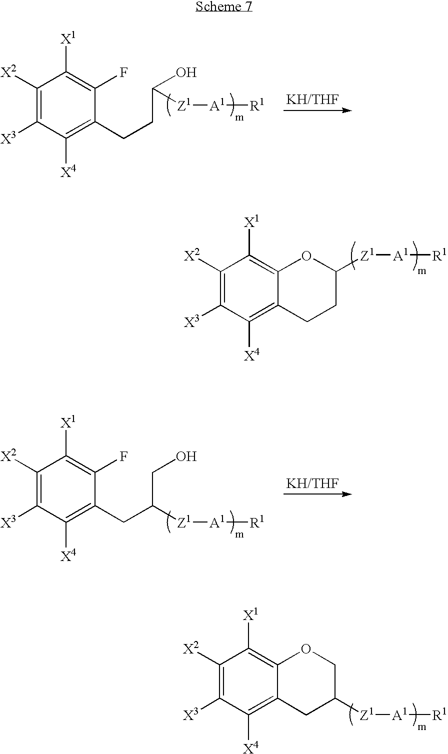

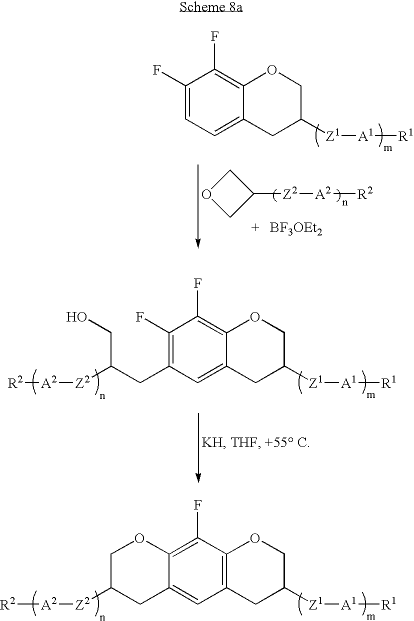

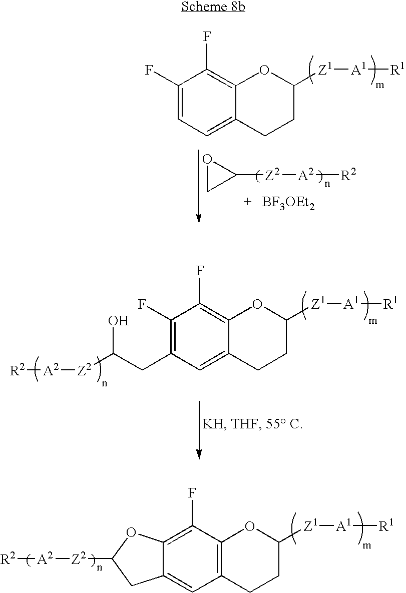

The products obtained in this way can, if desired, be re-employed as starting materials. In this way, compounds according to the invention having two heterocyclic rings can also be constructed given suitable fluorine substitution, as shown in the two reaction schemes 8a and 8b above.

The cyclisation reactions can be followed by further reactions, for example the functionalisation of the aromatic radical by introduction of further halogen substituents, such as, for example, chlorine, bromine or iodine, or by introduction of boronic acid groups by processes known from the literature.

Corresponding reaction examples are shown by scheme 9 below:

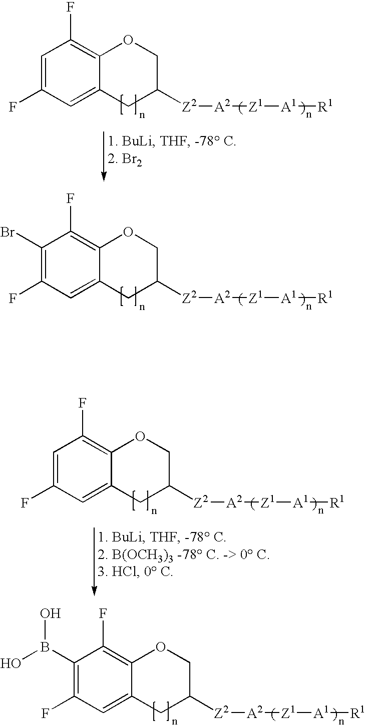

A preferred synthesis for the construction of aryl-substituted fluorobenzo-chromane derivatives of the general formula (Ia) is carried out by Suzuki coupling of corresponding boronic acids or boronic acid esters with 7-bromo-8-fluorochromanes or 7-bromo-6,8-difluorochromanes in accordance with scheme 10 below. The requisite boronic acid derivatives are prepared from bromene-substituted precursors by known methods, as disclosed, for example, in J. Org. Chem. 1995, 60, 7508-7510. The synthesis can be adapted to the compounds of the general formula (Ia) desired in each case through the choice of suitable starting materials. In this way, the particularly preferred compounds of the sub-formulae (Ia1a) and (Ia2b), inter alia, can be prepared.

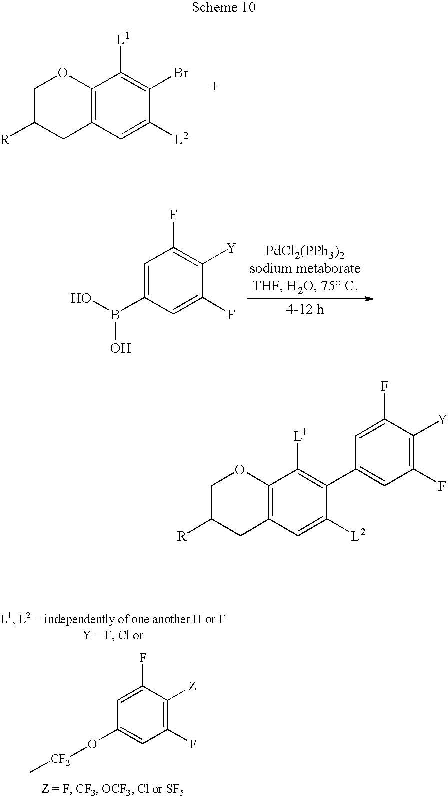

The reactions shown should only be regarded as illustrative. The person skilled in the art will be able to carry out corresponding variants of the syntheses presented and also to carry out other suitable synthetic methods in order to obtain the compounds of the formulae (I) to (VI) according to the invention.

The syntheses of various chromane derivatives according to the invention are, in addition, described by way of example in the examples.

The present invention also relates to liquid-crystalline media comprising from 2 to 40, preferably from 4 to 30, components as further constituents besides one or more compounds of the formulae (I) to (VI) according to the invention. These media particularly preferably comprise from 7 to 25 components besides one or more compounds according to the invention. These further constituents are preferably selected from nematic or nematogenic (monotropic or isotropic) substances, in particular substances from the classes of the azoxybenzenes, benzylideneanilines, biphenyls, terphenyls, 1,3-dioxanes, 2,5-tetrahydropyrans, phenyl or cyclohexyl benzoates, phenyl or cyclohexyl esters of cyclohexanecarboxylic acid, phenyl or cyclohexyl esters of cyclohexylbenzoic acid, phenyl or cyclohexyl esters of cyclohexylcyclohexanecarboxylic acid, cyclohexylphenyl esters of benzoic acid, of cyclohexanecarboxylic acid or of cyclohexylcyclohexanecarboxylic acid, phenylcyclohexanes, cyclohexylbiphenyls, phenylcyclohexylcyclohexanes, cyclohexylcyclohexanes, cyclohexylcyclohexylcyclohexenes, 1,4-biscyclohexylbenzenes, 4,4′-biscyclohexylbiphenyls, phenyl- or cyclohexylpyrimidines, phenyl- or cyclohexylpyridines, phenyl- or cyclohexyldioxanes, phenyl- or cyclohexyl-1,3-dithianes, 1,2-diphenylethanes, 1,2-dicyclohexylethanes, 1-phenyl-2-cyclohexylethanes, 1-cyclohexyl-2-(4-phenylcyclohexyl)ethanes, 1-cyclohexyl-2-biphenylethanes, 1-phenyl-2-cyclohexylphenylethanes, optionally halogenated stilbenes, benzyl phenyl ethers, tolans and substituted cinnamic acids. The 1,4-phenylene groups in these compounds may also be mono- or polyfluorinated.

The most important compounds suitable as further constituents of the media according to the invention can be characterised by the formulae 1, 2, 3, 4, 5 and 6:
R′-L-E-R″ 1
R′-L-COO-E-R″ 2
R′-L-OOC-E-R″ 3
R′-L-CH₂CH₂-E-R″ 4
R′-L-C≡C-E-R″ 5
R′-L-CF₂O-E-R″ 6

In the formulae 1, 2, 3, 4, 5 and 6, L and E, which may be identical or different, each, independently of one another, denote a divalent radical from the group formed by -Phe-, -Cyc-, -Phe-Phe-, -Phe-Cyc-, -Cyc-Cyc-, -Pyr-, -Dio-, -Thp-, -G-Phe- and -G-Cyc- and their mirror images, where Phe denotes unsubstituted or fluorine-substituted 1,4-phenylene, Cyc denotes trans-1,4-cyclohexylene or 1,4-cyclohexenylene, Pyr denotes pyrimidine-2,5-diyl or pyridine-2,5-diyl, Dio denotes 1,3-dioxane-2,5-diyl, Thp denotes tetrahydropyran-2,5-diyl and G denotes 2-(trans-1,4-cyclohexyl)ethyl, pyrimidine-2,5-diyl, pyridine-2,5-diyl, 1,3-dioxane-2,5-diyl or tetrahydropyran-2,5-diyl.

One of the radicals L and E is preferably Cyc or Phe. E is preferably Cyc, Phe or Phe-Cyc. The media according to the invention preferably comprise one or more components selected from the compounds of the formulae 1, 2, 3, 4, 5 and 6 in which L and E are selected from the group consisting of Cyc and Phe and simultaneously one or more components selected from the compounds of the formulae 1, 2, 3, 4, 5 and 6 in which one of the radicals L and E is selected from the group consisting of Cyc and Phe and the other radical is selected from the group consisting of -Phe-Phe-, -Phe-Cyc-, -Cyc-Cyc-, -G-Phe- and -G-Cyc-, and optionally one or more components selected from the compounds of the formulae 1, 2, 3, 4, 5 and 6 in which the radicals L and E are selected from the group consisting of -Phe-Cyc-, -Cyc-Cyc-, -G-Phe- and -G-Cyc-.

R′ and/or R″ each, independently of one another, denote alkyl, alkenyl, alkoxy, alkoxyalkyl, alkenyloxy or alkanoyloxy having up to 8 C atoms, —F, —Cl, —CN, —NCS, —(O)ᵢCH₃₋₍ₖ₊₁₎FₖClₗ, where i is 0 or 1, k and l, independently of one another, identically or differently, are 0, 1, 2 or 3, but with the proviso that the sum (k+l) is 1, 2 or 3.

In a smaller sub-group of the compounds of the formulae 1, 2, 3, 4, 5 and 6, R′ and R″ each, independently of one another, denote alkyl, alkenyl, alkoxy, alkoxyalkyl, alkenyloxy or alkanoyloxy having up to 8 C atoms. This smaller sub-group is called group A below, and the compounds are referred to by the sub-formulae 1a, 2a, 3a, 4a, 5a and Ga. In most of these compounds, R′ and R″ are different from one another, one of these radicals usually being alkyl, alkenyl, alkoxy or alkoxyalkyl.

In the smaller sub-group of the compounds of the formulae 1, 2, 3, 4, 5 and 6, which is known as group A, E in a preferred embodiment denotes

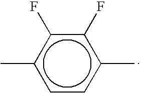

In another smaller sub-group of the compounds of the formulae 1, 2, 3, 4, 5 and 6, which is known as group B, R″ denotes —F, —Cl, —NCS or —(O)ᵢCH₃₋₍ₖ₊ₗ₎FₖClₗ, where i is 0 or 1, k and l, independently of one another, identically or differently, are 0, 1, 2 or 3, but with the proviso that the sum (k+l) is 1, 2 or 3. The compounds in which R″ has this meaning are referred to by the sub-formulae 1b, 2b, 3b, 4b, 5b and 6b. Particular preference is given to those compounds of the sub-formulae 1b, 2b, 3b, 4b, 5b and 6b in which R″ has the meaning —F, —Cl, —NCS, —CF₃, —OCHF₂ or —OCF₃.

In the compounds of the sub-formulae 1b, 2b, 3b, 4b, 5b and 6b, R′ has the meaning indicated for the compounds of the sub-formulae 1a to 6a and is preferably alkyl, alkenyl, alkoxy or alkoxyalkyl.

In a further smaller sub-group of the compounds of the formulae 1, 2, 3, 4, 5 and 6, R″ denotes —CN. This sub-group is referred to below as group C, and the compounds of this sub-group are correspondingly described by sub-formulae 1c, 2c, 3c, 4c, 5c and 6c. In the compounds of the sub-formulae 1c, 2c, 3c, 4c, 5c and 6c, R′ has the meaning indicated for the compounds of the sub-formulae 1a to 6a and is preferably alkyl, alkenyl, alkoxy or alkoxyalkyl.

Besides the preferred compounds of groups A, B and C, other compounds of the formulae 1, 2, 3, 4, 5 and 6 having other variants of the proposed substituents are also customary. All these substances are obtainable by methods which are known from the literature or analogously thereto.

Besides compounds of the formulae (I), (II), (III), (IV), (V) and/or (VI) according to the invention, the media according to the invention preferably comprise one or more compounds selected from groups A, B and/or C. The proportions by weight of the compounds from these groups in the media according to the invention are preferably:
group A: from 0 to 90%, preferably from 20 to 90%, particularly preferably from 30 to 90%;
group B: from 0 to 80%, preferably from 10 to 80%, particularly preferably from 10 to 70%;
group C: from 0 to 80%, preferably from 5 to 80%, particularly preferably from 5 to 50%;
   where the sum of the proportions by weight of the group A, B and/or C compounds present in the respective media according to the invention is preferably from 5 to 90% and particularly preferably from 10 to 90%.

The media according to the invention preferably comprise from 1 to 40%, particularly preferably from 5 to 30%, of the compounds according to the invention. Preference is furthermore given to media comprising more than 40%, particularly preferably from 45 to 90%, of compounds according to the invention. The media preferably comprise one, two, three, four or five compounds according to the invention.

Examples of the compounds of the formulae 1, 2, 3, 4, 5 and 6 are the compounds shown below: 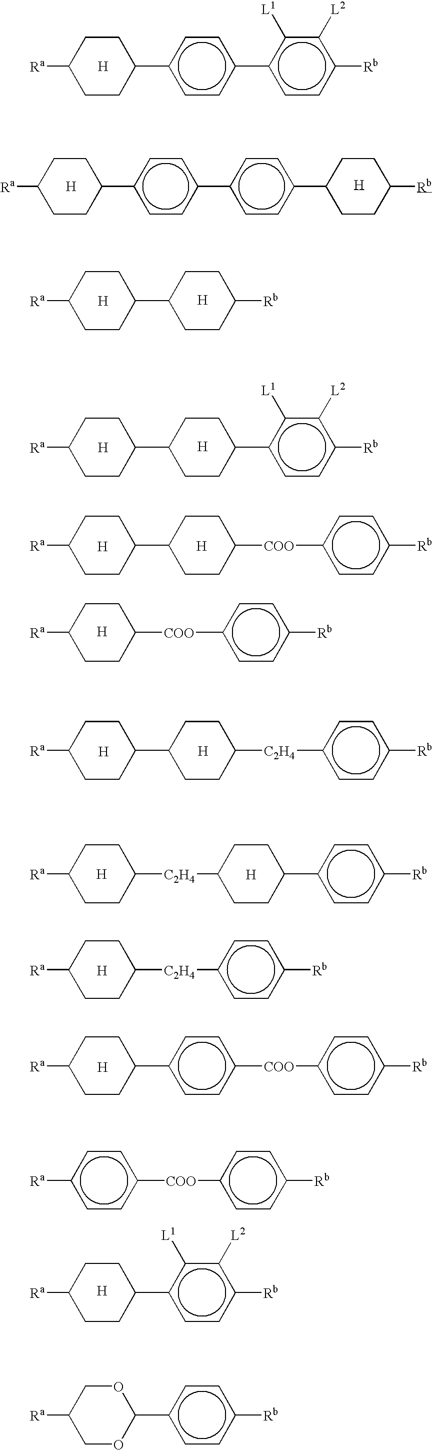 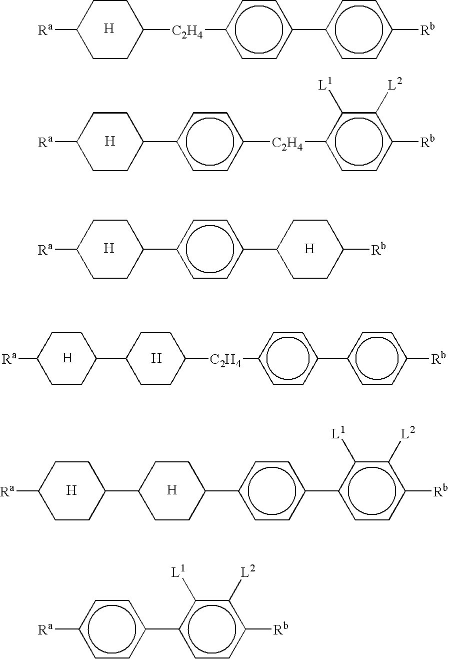
where R^{a}, R^{b}, independently of one another, denote —CₚH₂ₚ₊₁ or —OCₚH₂ₚ₊₁ and p 1, 2, 3, 4, 5, 6, 7 or 8, and L¹, L², independently of one another, denote —H or —F 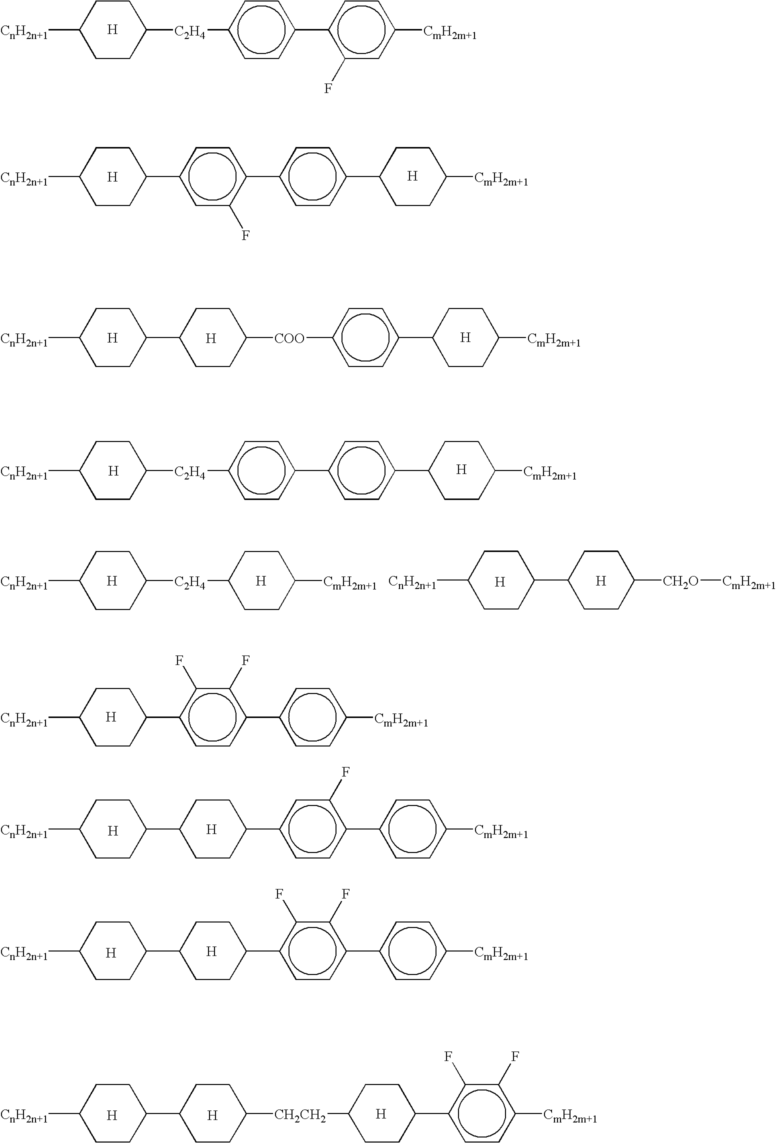 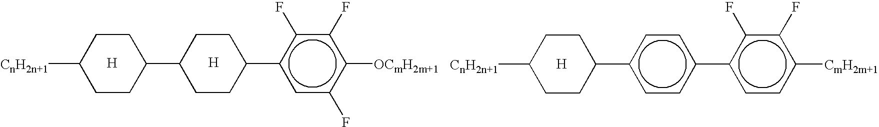
where m, n, independently of one another, denote 1, 2, 3, 4, 5, 6, 7 or 8.

The liquid-crystal mixtures according to the invention are prepared in a manner which is conventional per se. In general, the desired amount of the components used in lesser amount is dissolved in the components making up the principal constituent, preferably at elevated temperature. It is also possible to mix solutions of the components in an organic solvent, for example in acetone, chloroform or methanol, and to remove the solvent again, for example by distillation, after thorough mixing. It is furthermore possible to prepare the mixtures in other conventional manners, for example by using premixes, for example homologue mixtures, or using so-called “multibottle” systems.

The dielectrics may also comprise further additives known to the person skilled in the art and described in the literature. For example, from 0 to 15%, preferably from 0 to 10%, of pleochroic dyes and/or chiral dopants can be added. The individual compounds added are employed in concentrations of from 0.01 to 6%, preferably from 0.1 to 3%. However, the concentration data of the other constituents of the liquid-crystal mixtures, i.e. the liquid-crystalline or mesogenic compounds, are indicated without taking into account the concentration of these additives.

In the present application and in the following examples, the structures of the liquid-crystal compounds are indicated by means of acronyms, the transformation into chemical formulae taking place in accordance with Tables A and B below. All radicals CₙH₂ₙ₊₁ and CₘH₂ₘ₊₁ are straight-chain alkyl radicals having n and m C atoms respectively. n and m denote integers, preferably 1, 2, 3, 4, 5, 6, 7, 8, 9 or 10, where n=m or n≠m. The coding in Table B is self-evident. In Table A, only the acronym for the parent structure is indicated. In individual cases, the acronym for the parent structure is followed, separated by a dash, by a code for the substituents R^{1*}, R^{2*}, L^{1*} and L^{2*}:

| Code for R¹*, | | | | |
|---|---|---|---|---|
| R²*, L¹*, L²* | R¹* | R²* | L¹* | L²* |
| nm | CₙH₂ₙ₊₁ | CₘH₂ₘ₊₁ | H | H |
| nOm | CₙH₂ₙ₊₁ | OCₘH₂ₘ₊₁ | H | H |
| nO•m | OCₙH₂ₙ₊₁ | CₘH₂ₘ₊₁ | H | H |
| n | CₙH₂ₙ₊₁ | CN | H | H |
| nN•F | CₙH₂ₙ₊₁ | CN | F | H |
| nN•F•F | CₙH₂ₙ₊₁ | CN | F | F |
| nF | CₙH₂ₙ₊₁ | F | H | H |
| nOF | OCₙH₂ₙ₊₁ | F | H | H |
| nF•F | CₙH₂ₙ₊₁ | F | F | H |
| nmF | CₙH₂ₙ₊₁ | CₘH₂ₘ₊₁ | F | H |
| nOCF₃ | CₙH₂ₙ₊₁ | OCF₃ | H | H |
| n-Vm | CₙH₂ₙ₊₁ | —CH═CH—CₘH₂ₘ₊₁ | H | H |
| nV-Vm | CₙH₂ₙ₊₁—CH═CH— | —CH═CH—CₘH₂ₘ₊₁ | H | H |

Preferred mixture components are given in Tables A and B.

TABLE A

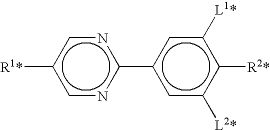

PYP

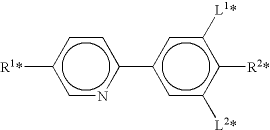

PYRP

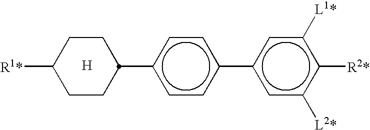

BCH

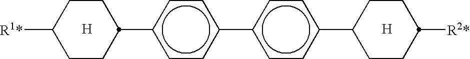

CBC

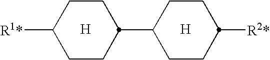

CCH

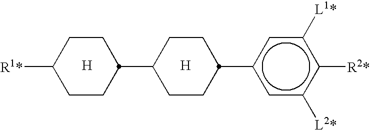

CCP

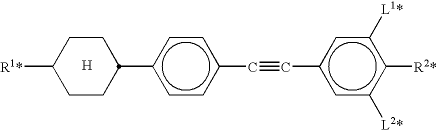

CPTP

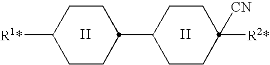

CCN

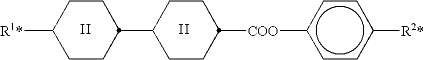

CP

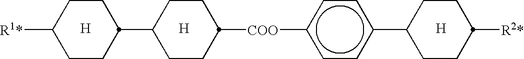

CCPC

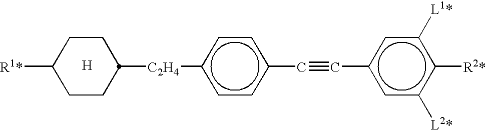

CEPTP

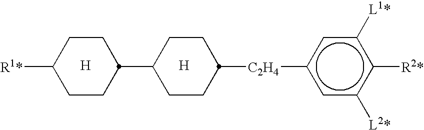

ECCP

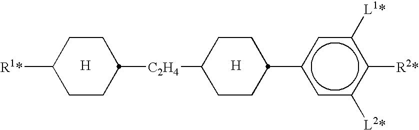

CECP

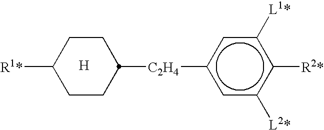

EPCH

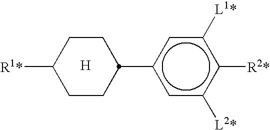

PCH

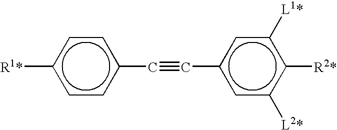

PTP

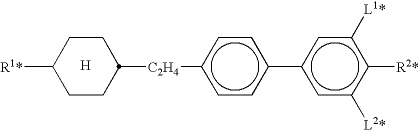

BECH

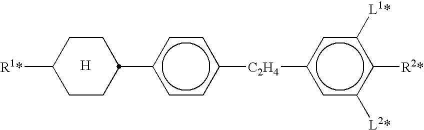

EBCH

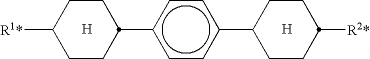

CPC

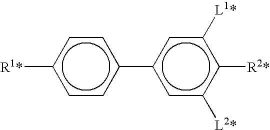

B

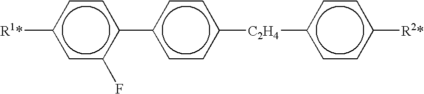

FET-nF

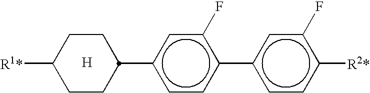

CGG

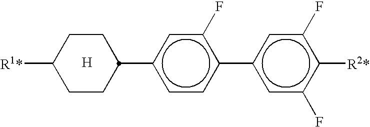

CGU

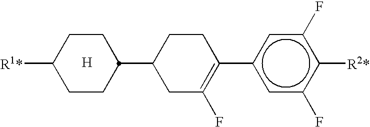

CFU

TABLE B

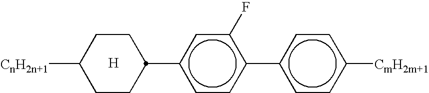

BCH-n.Fm

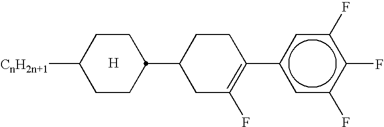

CFU-n-F

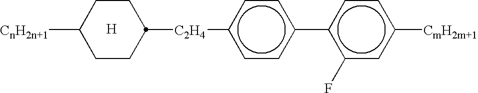

I-nm

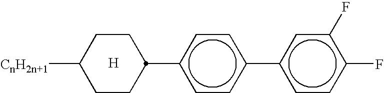

BCH-nF.F

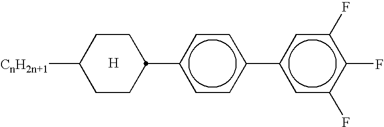

BCH-nF.F.F

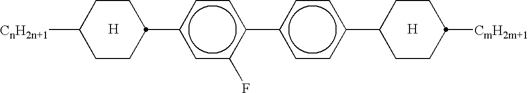

CBC-nmF

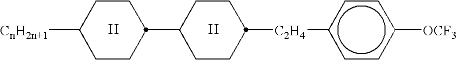

ECCP-nOCF₃

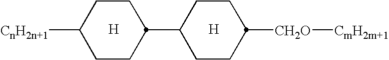

CCH-n1Em

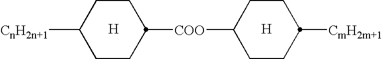

OS-nm

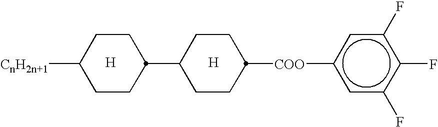

CCZU-n-F

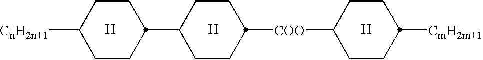

CH-nm

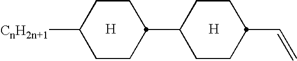

CC-n-V

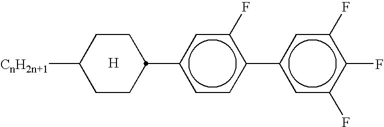

CGU-n-F

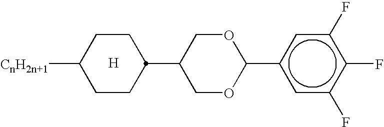

CDU-n-F

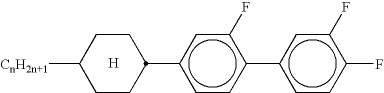

CGG-n-F

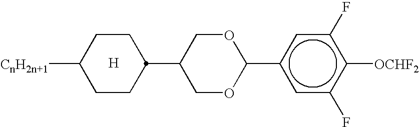

CDU-n-OD

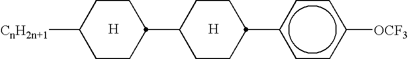

CCP-nOCF₃

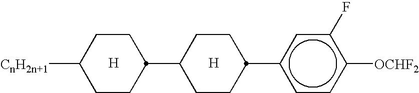

CCP-nOCF₂.F

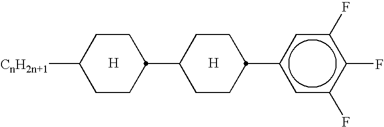

CCP-nF.F.F

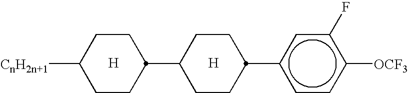

CCP-nOCF₃.F

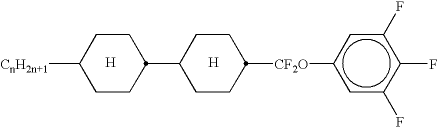

CCQU-n-F

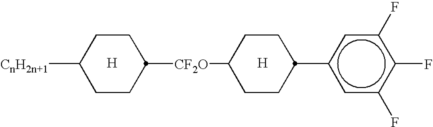

CQCU-n-F

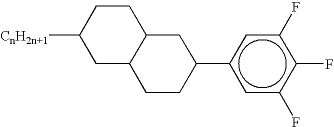

Dec-U-n-F

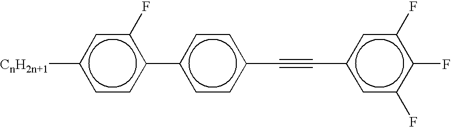

GPTU-n-F

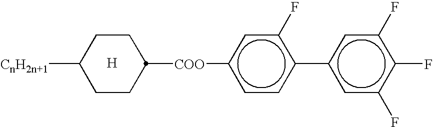

CZGU-n-F

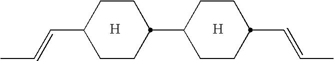

CC-1V-V1

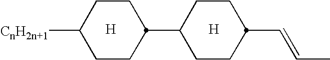

CC-n-V1

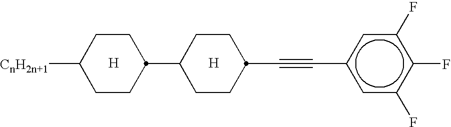

CCTU-n-F

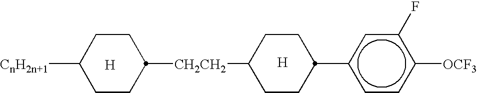

CECG-n-OT

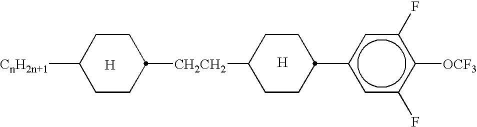

CECU-n-OT

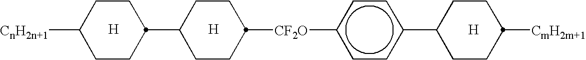

CCQPC-n-m

TABLE C
Table C shows possible dopants which are preferably added to the mix-
tures according to the invention.

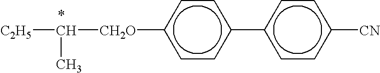

C 15

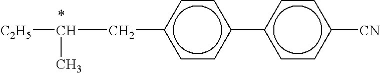

CB 15

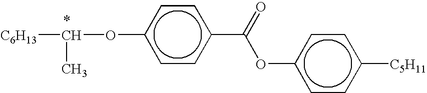

CM 21

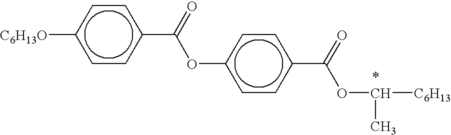

CM 33

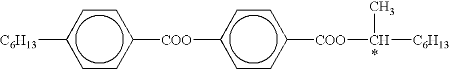

R/S 811

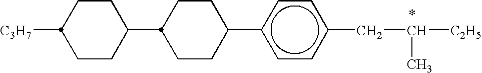

CM 44

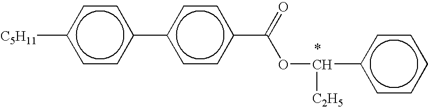

CM 45

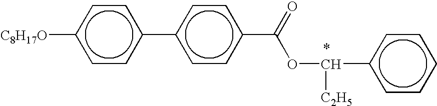

CM 47

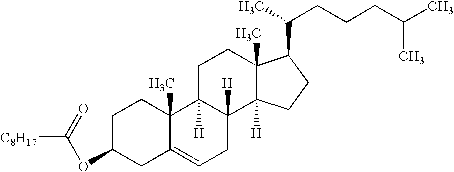

CN

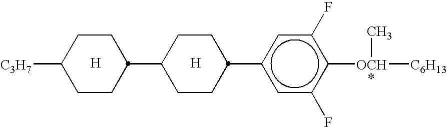

R/S 2011

Particular preference is given to mixtures according to the invention which, besides one or more compounds of the formulae (I), (II), (III), (IV), (V) and/or (VI), comprise two, three or more compounds selected from Tables A and/or B.

The following examples are intended to explain the invention without restricting it. Above and below, percentages denote percent by weight. All temperatures are indicated in degrees Celsius. Tg is the glass transition temperature and cl.p. is the clearing point. Furthermore, C=crystalline state, N=nematic phase, Sm=smectic phase and I=isotropic phase. The data between these symbols represent the transition temperatures. Δn denotes optical anisotropy (589 nm, 20° C.), Δ∈ denotes the dielectric anisotropy (1 kHz, 20° C.) and 71 denotes the rotational viscosity at 20° C. [mPas].

The Δn and Δ∈ values of the compounds according to the invention were obtained by extrapolation from liquid-crystalline mixtures which consisted of 10% of the respective compound according to the invention and 90% either of the commercially available liquid crystal ZLI 4792 (Δn and positive Δ∈ values) or the likewise commercially available liquid crystal ZLI 2857 (negative Δ∈ values), both Merck, Darmstadt.

Above and below, the following abbreviations are used:

AIBN azoisobutyronitrile

BuLi butyllithium

DCM dichloromethane

EA ethyl acetate

KH potassium hydride

KHMDS potassium hexamethyldisilazane

LDA lithium diisopropylamide

MCPBA 3-chloroperoxybenzoic acid

MTBE tert-butyl methyl ether

NBS N-bromosuccinimide

FT room temperature

THF tetrahydrofuran

TMEDA tetramethylethylenediamine

### EXAMPLE 1

### 7,8-Difluoro-6-(4-trans-pentylcyclohexyl)chromane

### 1st Step: 2-Allyloxy-3,4-difluoro-5-(4-trans-pentylcyclohexyl)benzaldehyde

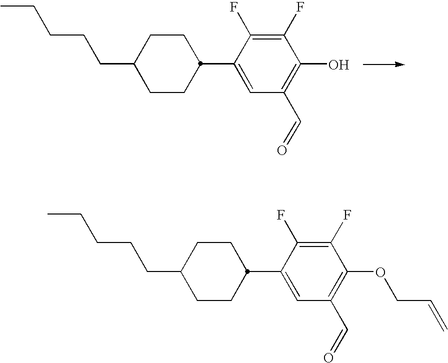

3.00 g (9.67 mmol) of 3,4-difluoro-2-hydroxy-5-(4-pentylcyclohexyl)benzaldehyde are dissolved in 30 ml of acetone and, after addition of 2.1 g (15 mmol) of potassium carbonate and 2.5 ml (30 mmol) of allyl bromide, warmed at 60° C. for 3 hours. After filtration, the filtrate is evaporated, and the residue is purified by chromatography on silica gel using heptane/MTB ether (50:1), giving 3.23 g (95%) of 2-allyloxy-3,4-difluoro-5-(4-pentylcyclohexyl)benzaldehyde as colourless crystals.

### 2nd Step: 2-Allyloxy-3,4-difluoro-5-(4-trans-pentylcyclohexyl)-1-vinylbenzene

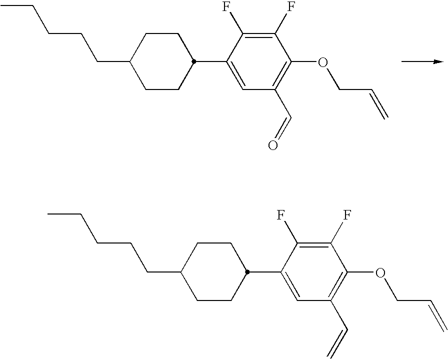

3.66 g (10.0 mmol) of methyltriphenylphosphonium bromide are initially introduced in 20 ml of THF, and 1.15 g (10.0 mmol) of potassium tert-butoxide are added with ice cooling. After 10 minutes, a solution of 3.23 g (9.22 mmol) of 2-allyloxy-3,4-difluoro-5-(4-pentylcyclohexyl)benzaldehyde in 15 ml of THF is added dropwise. The cooling is removed, the batch is left to stir at room temperature for 2 hours and added to ice-water. The aqueous phase is separated off and extracted three times with MTB ether. The combined organic phases are washed with water and saturated sodium chloride solution and evaporated under reduced pressure. The residue is taken up in heptane and filtered through silica gel using heptane/MTB ether (50:1), giving 2.71 g (84%) of 2-allyloxy-3,4-difluoro-5-(4-pentylcyclohexyl)-1-vinylbenzene as colourless oil.

### 3rd Step: 7,8-Difluoro-6-(4-trans-pentylcyclohexyl)-2H-chromene

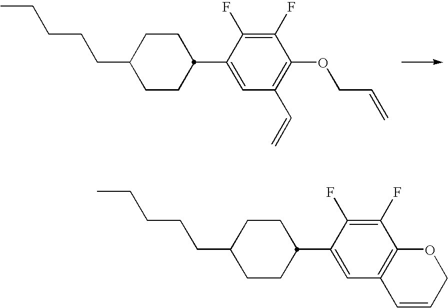

2.68 g of 2-allyloxy-3,4-difluoro-5-(4-trans-pentylcyclohexyl)-1-vinylbenzene are dissolved in 40 ml of dichloromethane under nitrogen and, after addition of 63 mg of Grubbs catalyst, left to stir at room temperature for 2 hours. The batch is evaporated, and the residue is chromatographed on silica gel using n-heptane/MTB ether (50:1), giving 930 mg (38%) of 7,8-difluoro-6-(4-pentylcyclohexyl)-2H-chromene as colourless crystals.

### 4th Step: 7,8-Difluoro-6-(4-trans-pentylcyclohexyl)chromane

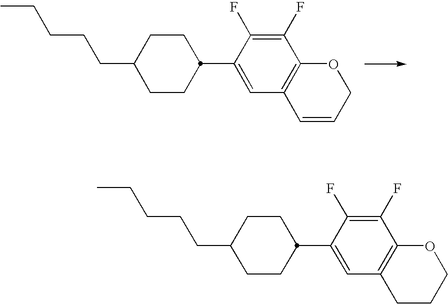

930 mg of 7,8-difluoro-6-(4-pentylcyclohexyl)-2H-chromene are hydrogenated to completion in ethanol on palladium/activated carbon (5%) at 1 bar and room temperature. The catalyst is filtered off, the filtrate is evaporated, and the residue is recrystallised from ethanol, giving 680 mg (79%) of 7,8-difluoro-6-(4-trans-pentylcyclohexyl)chromane as colourless crystals of m.p. 49° C.

Δ∈=−3.2

Δn=0.0685

### EXAMPLE 2

### 2-Ethyl-6-(4-trans-ethylcyclohexyl)-7,8-difluorochromane

### 1st Step: 2,3-Difluoro-4-(4-trans-ethylcyclohexyl)-6-vinylphenol

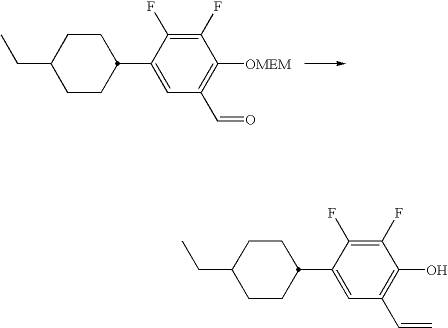

26.5 g (74.4 mmol) of methyltriphenylphosphonium bromide are initially introduced in 150 ml of THF, and a solution of 8.33 g (74.2 mmol) of potassium tert-butoxide in 50 ml of THF is added dropwise with ice cooling. After 2 hours, 30.0 g (67.5 mmol) of 3,4-difluoro-2-(2-methoxyethoxymethoxy)-5-(4-trans-ethylcyclohexyl)benzaldehyde in 50 ml of THF are added, and the mixture is left to stir overnight at room temperature. The batch is hydrolysed using water, 100 ml of conc. hydrochloric acid are added, and the mixture is left to stir vigorously for 3 hours. The mixture is subsequently extracted with MTBE, the combined extracts are washed with saturated sodium chloride solution, dried over sodium sulfate and evaporated, giving 16.0 g (81%) of 2,3-difluoro-4-(4-trans-ethylcyclohexyl)-6-vinylphenol as yellow oil which is sufficiently pure for further reactions.

### 2nd Step: 2-(1-Ethylallyloxy)-5-(4-trans-ethylcyclohexyl)-3,4-difluoro-1-vinylbenzene

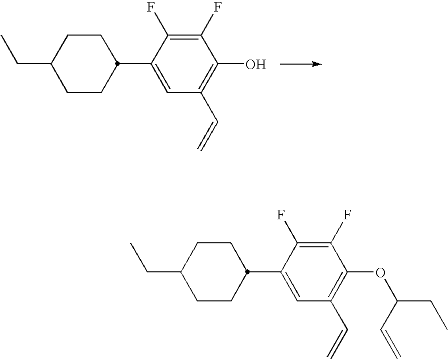

7.84 g (45.0 mmol) of diethyl azodicarboxylate in 50 ml of THF are added dropwise to a solution of 10.0 g (37.5 mmol) of 4-(4-trans-ethylcyclohexyl)-2,3-difluoro-6-vinylphenol, 11.8 g (45.0 mmol) of triphenylphosphine and 3.88 g (45.0 mmol) of 1-penten-3-ol in 150 ml of THF. The mixture is left to stir at room temperature for 5 hours and extracted with ethyl acetate. The combined organic phases are washed with saturated sodium chloride solution and dried over sodium sulfate. The solvent is removed under reduced pressure, and the crude product is purified by chromatography on silica gel using n-heptane/ethyl acetate (20:1), giving 9.91 g (79%) of 2-(1-ethylallyloxy)-5-(4-trans-ethylcyclohexyl)-3,4-difluoro-1-vinylbenzene as colourless oil.

### 3rd Step: 2-Ethyl-6-(4-trans-ethylcyclohexyl)-7,8-difluoro-2H-chromene

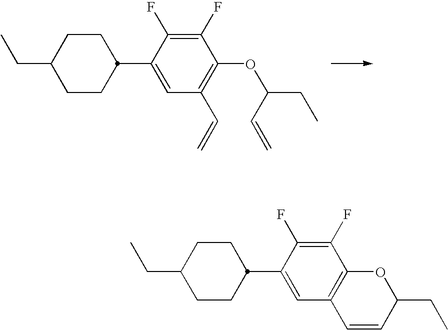

Analogously to the synthesis described in Example 1, in the 3rd step, 7.50 g (22.4 mmol) of 2-(1-ethylallyloxy)-5-(4-trans-ethylcyplohexyl)-3,4-difluoro-1-vinylbenzene give 5.29 g (77%) of 2-ethyl-6-(4-trans-ethylcyclohexyl)-7,8-difluoro-2H-chromene as colourless crystals.

### 4th Step: 2-Ethyl-6-(4-trans-ethylcyclohexyl)-7,8-difluorochromane

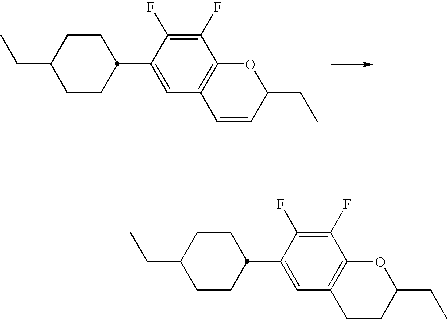

5.00 g (16.3 mmol) of 2-ethyl-6-(4-trans-ethylcyclohexyl)-7,8-difluoro-2H-chromene are dissolved in 20 ml of THF and hydrogenated to completion in the presence of palladium/activated carbon catalyst. The solution is filtered through silica gel, and the solvent is removed under reduced pressure, giving 4.38 g (87%) of 2-ethyl-6-(4-trans-ethylcyclohexyl)-7,8-difluorochromane as colourless crystals.

Δ∈=−5.5

Δn=0.112

### EXAMPLE 3

### 6-(4-Ethylphenyl)-7,8-difluoro-2-methylchromane

### 1 st Step: 4-(4′-Ethyl-5,6-difluoro-4-hydroxybiphenyl-3-yl)but-3-en-2-one

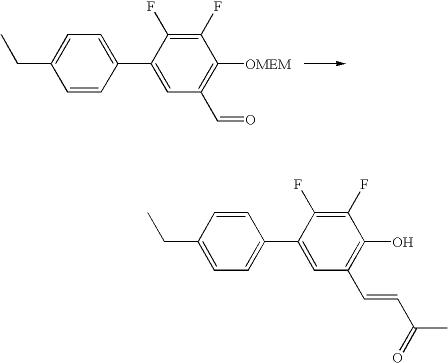

9.44 g (26.9 mmol) of 4′-ethyl-5,6-difluoro-4-(2-methoxyethoxymethoxy)biphenyl-3-carbaldehyde are dissolved in 50 ml of acetone, 8.5 g of 50 percent sodium hydroxide solution and 300 ml of water are added, and the mixture is left to stir at room temperature for 3 days. The batch is extracted with dichloromethane, evaporated and taken up in 100 ml of THF. After addition of 30 ml of conc. hydrochloric acid, the mixture is stirred vigorously overnight and subsequently extracted with MTBE. The combined organic phases are washed with water and dried over sodium sulfate, and the solvent is removed under reduced pressure. Filtration through silica gel using MTBE gives 12.2 g (92%) of 4-(4′-ethyl-5,6-difluoro-4-hydroxybiphenyl-3-yl)but-3-en-2-one as colourless oil.

### 2nd Step: 4-(4′-Ethyl-5,6-difluoro-4-hydroxybiphenyl-3-yl)butan-2-one

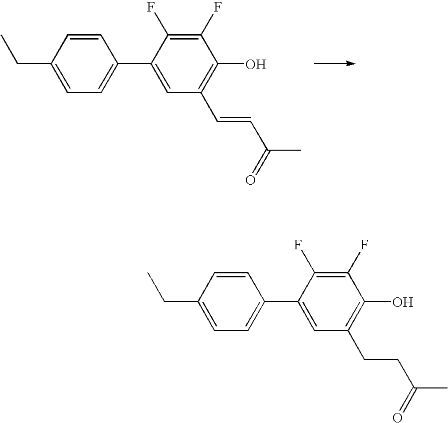

10.1 g (33.3 mmol) of 4-(4′-ethyl-5,6-difluoro-4-hydroxybiphenyl-3-yl)but-3-en-2-one are dissolved in 80 ml of THF and hydrogenated to completion on palladium/activated carbon (5%). The mixture is subsequently filtered and evaporated, and the residue is purified by chromatography on silica gel, giving 7.71 g (76%) of 4-(4′-ethyl-5,6-difluoro-4-hydroxybiphenyl-3-yl)butan-2-one as colourless oil.

### 3rd Step: 4′-Ethyl-2,3-difluoro-5-(3-hydroxybutyl)biphenyl-4-ol

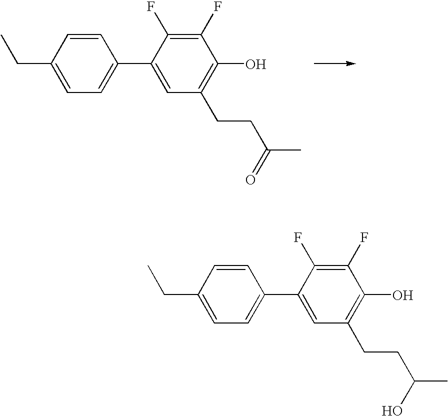

5.8 g (19.1 mmol) of 4-(4′-ethyl-5,6-difluoro-4-hydroxybiphenyl-3-yl)butan-2-one are dissolved in 30 ml of isopropanol, 600 mg (15.9 mmol) of sodium borohydride are added, and the mixture is stirred overnight at room temperature. The batch is carefully acidified, diluted with 50 ml of water and extracted with MTBE. The combined organic phases are washed with water and dried over sodium sulfate. Removal of the solvent under reduced pressure gives 4.91 g (84%) of 4′-ethyl-2,3-difluoro-5-(3-hydroxybutyl)biphenyl-4-ol as yellow oil which can be reacted without further purification.

### 4th Step: 6-(4-Ethylphenyl)-7,8-difluoro-2-methylchromane

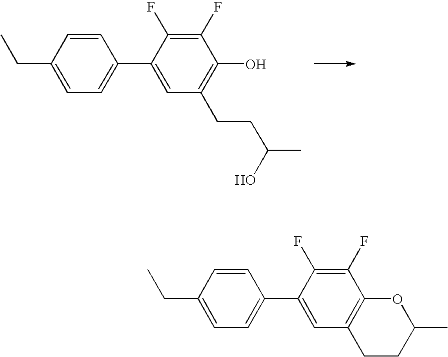

4.5 g (14.7 mmol) of 4′-ethyl-2,3-difluoro-5-(3-hydroxybutyl)biphenyl-4-ol are dissolved in 25 ml of glacial acetic acid and 25 ml of 50 percent sulfuric acid and warmed at 60° C. for 30 minutes. The batch is added to ice-water, neutralised using sodium hydroxide solution and extracted with MTBE. The combined organic phases are dried over sodium sulfate and evaporated, and the residue is purified by chromatography on silica gel, giving 3.05 g (72%) of 6-(4-ethylphenyl)-7,8-difluoro-2-methylchromane as colourless solid.

Δ∈=−8.7

Δn=0.191

### EXAMPLE 4

### 7,8-Difluoro-2-methyl-6-(4-trans-propylcyclohexyl)chromane

### 1st Step: 2,3-Difluoro-6-(3-hydroxybutyl)-4-(4-trans-propylcyclohexyl)phenol

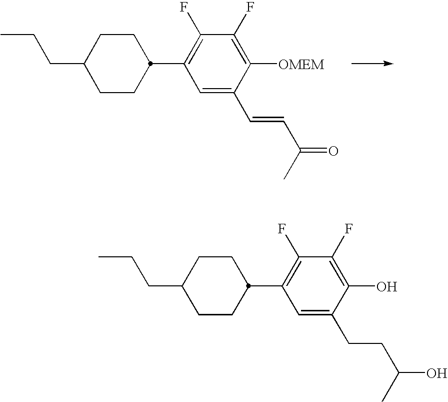

15.9 g (39 mmol) of 4-[3,4-difluoro-2-(2-methoxyethoxymethoxy)-5-(4-propylcyclohexyl)phenyl]but-3-en-2-one are hydrogenated to completion in 150 ml of tetrahydrofuran on palladium/activated carbon catalyst at 4 bar and room temperature. The solution is filtered, 150 ml of methanol and 15 ml of conc. hydrochloric acid are added, and the mixture is left to stir overnight at room temperature. The batch is subsequently added to water and extracted three times with MTB ether. The combined organic phases are washed with water and dried over sodium sulfate. The solvent is removed under reduced pressure and the residue is chromatographed on silica gel using heptane/MTB ether (1:1), giving 12.3 g (77%) of 2,3-difluoro-6-(3-hydroxybutyl)-4-(4-propylcyclohexyl)phenol as colourless oil.

### 2nd Step: 7,8-Difluoro-2-methyl-6-(4-trans-propylcyclohexyl)chromane

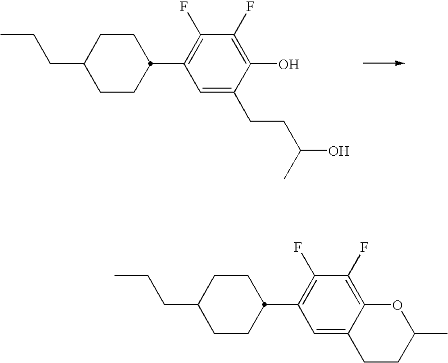

2.30 g (7.05 mmol) of 2,3-difluoro-6-(3-hydroxybutyl)-4-(4-propylcyclohexyl)phenol and 1.94 g (7.40 mmol) of triphenylphosphine are dissolved in 20 ml of tetrahydrofuran, and 1.64 ml (8.46 mmol) of diisopropyl azodicarboxylate in 10 ml of tetrahydrofuran are added dropwise. The batch is stirred overnight at room temperature, diluted with 30 ml of MTB ether and added to water. The organic phase is separated off and extracted three times with MTB ether. The combined organic phases are washed with water and dried over sodium sulfate. The solvent is removed under reduced pressure, and the residue is purified by chromatography on silica gel using heptane/MTB ether (4:1) and subsequently recrystallised from ethanol, giving 1.3 g (55%) of colourless crystals of m.p. 69° C.

Δ∈=−7.7

Δn=0.0759

### EXAMPLE 5

### 6-(4-trans-Ethylcyclohexyl)-7,8-difluoro-2-p-tolylchromane

### 1st Step: 5-(4-trans-Ethylcyclohexyl)-3,4-difluoro-2-(1-p-tolylallyloxy)benzaldehyde

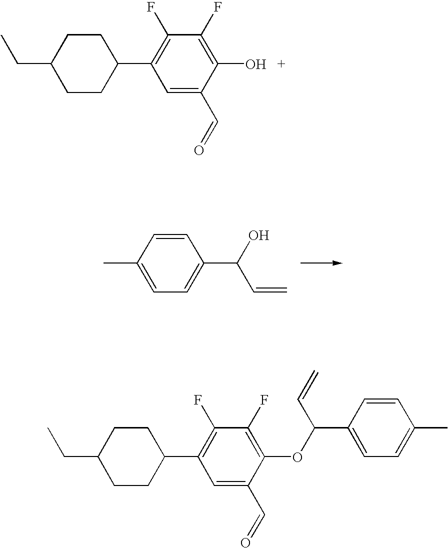

Analogously to the synthesis described under Example 2, the Mitsunobu reaction of 3,4-difluoro-2-hydroxy-5-(4-trans-pentylcyclohexyl)benzaldehyde and 1-p-tolylprop-2-en-1-ol gives 5-(4-trans-ethylcyclohexyl)-3,4-difluoro-2-(1-p-tolylallyloxy)benzaldehyde in 53 percent yield as colourless solid.

### 2nd Step: 1-(4-trans-Ethylcyclohexyl)-2,3-difluoro-4-(1-p-tolylallyloxy)-5-vinylbenzene

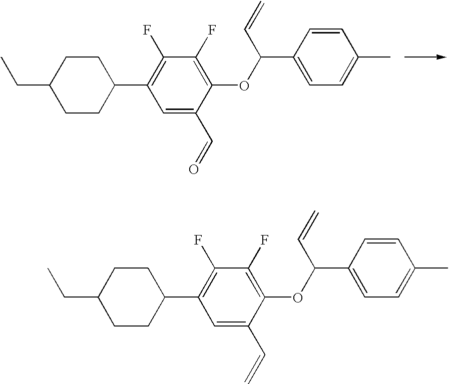

Analogously to the synthesis described under Example 1; the Wittig reaction of 5-(4-trans-ethylcyclohexyl)-3,4-difluoro-2-(1-p-tolylallyloxy)benzaldehyde with methylenetriphenyl-λ⁵-phosphane gives 1-(4-trans-ethylcyclohexyl)-2,3-difluoro-4-(1-p-tolylallyloxy)-5-vinylbenzene in 83 percent yield as colourless solid.

### 3rd Step: 6-(4-trans-Ethylcyclohexyl)-7,8-difluoro-2-p-tolyl-2H-chromene

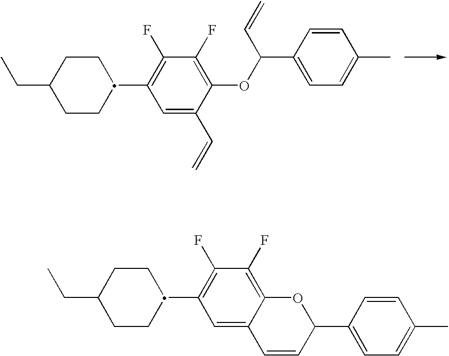

Analogously to the synthesis described in Example 1, ring-closure metathesis of 1-(4-trans-ethylcyclohexyl)-2,3-difluoro-4-(1-p-tolylallyloxy)-5-vinylbenzene gives 6-(4-trans-ethylcyclohexyl)-7,8-difluoro-2-p-tolyl-2H-chromene in 69 percent yield as colourless crystals.

### 4th Step: 6-(4-trans-Ethylcyclohexyl)-7,8-difluoro-2-p-tolylchromane

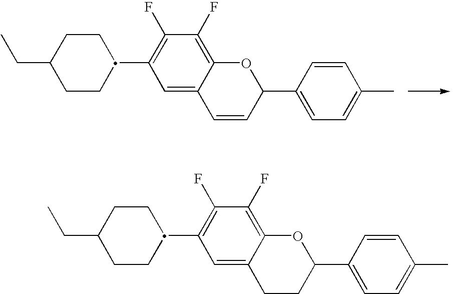

6-(4-trans-Ethylcyclohexyl)-7,8-difluoro-2-p-tolyl-2H-chromene is hydrogenated analogously to the synthesis described in Example 1, giving 6-(4-trans-ethylcyclohexyl)-7,8-difluoro-2-p-tolylchromane in 92 percent yield as colourless crystals.

Δ∈=−4.1

Δn=0.1561

### EXAMPLE 6

### trans-3-(4-Ethylcyclohexyl)oxetane

The compound of the following formula 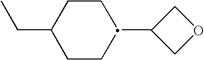
is prepared as follows:

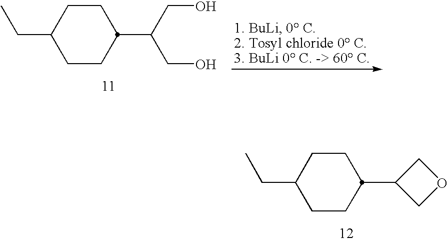

67.4 ml of BuLi (15% in hexane) are added dropwise at 0° C. to a solution of 20 g of diol (11) in 100 ml of THF. After 30 minutes, a solution of 19 g of tosyl chloride in 100 ml of THF is added dropwise (exothermic, warming to about 40° C.), and the resultant mixture is stirred at room temperature for 1 hour, before a further 67.4 ml of BuLi are added with ice cooling. The reaction is heated at 60° C. for 4 hours. The THF is removed in a rotary evaporator, the residue is treated with water and MTBE, the organic phase is separated off, dried and evaporated in a rotary evaporator. Purification of the residue by column chromatography (heptane/EA 6:1) gives 11.1 g of a colourless oil (12).

Yield: E 61%

The following compounds of Examples 7 and 8 are obtained analogously to Example 6 using the corresponding precursors:

### EXAMPLE 7

### trans-3-(4-n-Propylcyclohexyl)oxetane

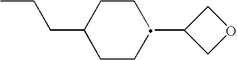

Yield: 68%

### EXAMPLE 8

### trans-3-(4′-n-Propylbicyclohexyl-4-yl)oxetane

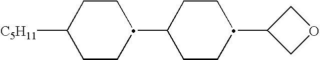

Yield: 57%

### EXAMPLE 9

### ORTHO-LITHIATION

### trans-2-(4-Ethylcyclohexyl)-3-(2,3,4-trifluorophenyl)propan-1-ol

The compound of the following formula 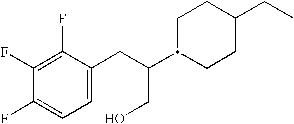
is prepared as follows:

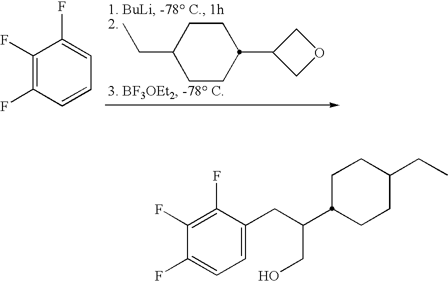

40 ml of BuLi (15% in hexane) are slowly added dropwise at −78° C. under nitrogen to a solution of 8.7 g of trifluorobenzene in 100 ml of THF, and the mixture is stirred at this temperature for a further 1 hour. Firstly 7.6 g of the oxetane mentioned (HPLC content 90% trans, 10% cis) are injected into the dark-yellow solution, and, after 15 minutes, 5.1 ml of BF₃ etherate are added dropwise. During addition of the BF₃ etherate, the mixture must be well cooled in order to keep the reaction temperature below 70° C. After stirring at −80° C. for a further 60 minutes (TLC monitoring, complete conversion), the reaction is quenched at −78° C. using 50 ml of ammonium chloride solution. MTBE is added to the thawed reaction mixture, the mixture is slightly acidified using 2N HCl, the aqueous phase is separated off and subsequently extracted a number of times with MTBE. The combined organic phases are washed with water and saturated sodium chloride solution, dried over sodium sulfate and evaporated in a rotary evaporator. Purification of the residue by chromatography over 500 ml of silica gel (eluent: toluene) gives 10.7 g of a colourless oil.

According to HPLC, the content of the desired trans compound is 81%.

Yield: 71%.

The following compounds of Examples 10 to 17 are obtained analogously to Example 9 using the corresponding oxetane precursors. The lithiated aromatic compound here is prepared by halogen-metal exchange, thus diethyl ether is used as solvent.

| Ex- | Starting | | | |
|---|---|---|---|---|
| ample | compound | Solvent | Compound | Yield [%] |
| 10 | 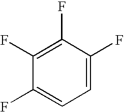 | THF | 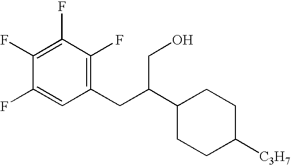 | 85 |
| 11 | 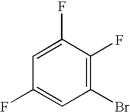 | Diethyl ether | 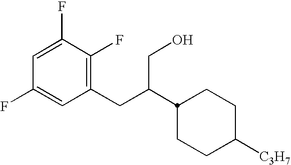 | 95 |
| 12 | 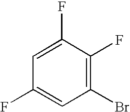 | Diethyl ether | 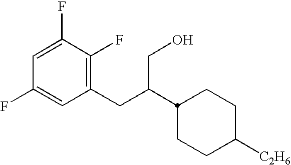 | 99 |
| 13 | 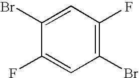 | Diethyl ether | 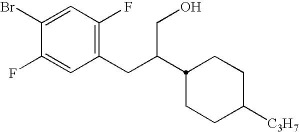 | 83 |
| 14 | 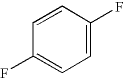 | THF | 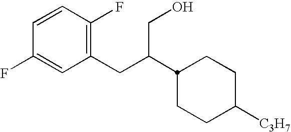 | 94 |
| 15 | 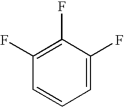 | Diethyl ether | 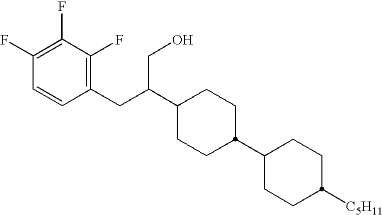 | 79 |
| 16 | 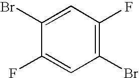 | DiethyI ether | 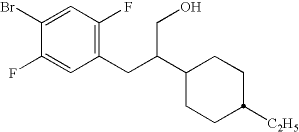 | 91 |
| 17 | 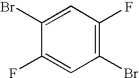 | Diethyl ether | 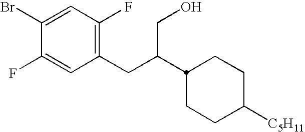 | 62 |

### EXAMPLE 18

### Cyclisation

### trans-7,8-Difluoro-3-(4-(ethyl)cyclohexyl)chromane

The compound of the following formula 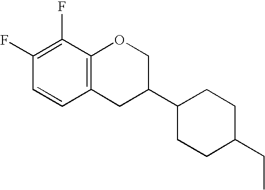
is prepared as follows:

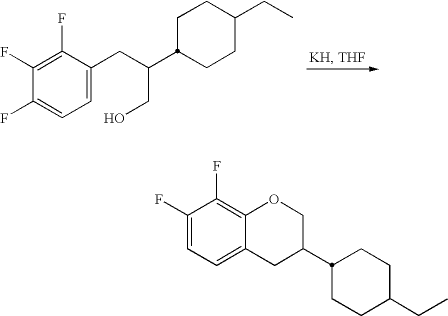

A solution of 10 g of the alcohol (content 95%) in 250 ml of THF is slowly added dropwise at 40° C. under N₂ to a suspension of 4.6 g of KH (30% in paraffin oil) in 500 ml of THF. After a further 2 hours at 55° C., the reaction is complete according to TLC monitoring. The reaction is quenched using 10 ml of saturated ammonium chloride solution, the majority of the THF is removed, toluene is added, the mixture is extracted with water, and the organic phase is separated off. The aqueous phase is subsequently extracted a further three times with toluene. The combined organic phases are dried over sodium sulfate and evaporated in a rotary evaporator, and the residue is purified by column chromatography (heptane/toluene 19:1), giving 6.8 g of a colourless solid.

Yield: 76%

Recrystallisation gives the pure trans compound:

C 82 I

Δn: 0.0729

Δ∈: 11.8

cl.p.: −9.1° C.

The following compounds of Examples 19 to 25 are obtained analogously using the corresponding precursors:

| | | Reaction |
|---|---|---|
| Ex- | | conds., |
| ample | Starting compound | solvent |
| 19 | 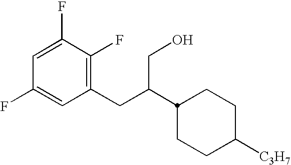 | 4h/40° C. KH/THF 5h/50° C. KH/THF |
| 20 | 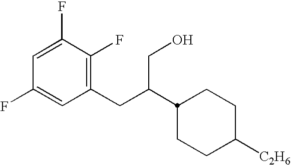 | 5h/60° C. KH/THF |
| 21 | 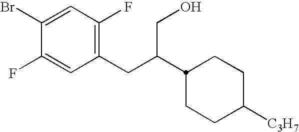 | 6h/50° C. KH/THF |
| 22 | 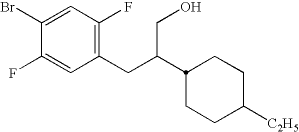 | 3h/50° C. KH/THF |
| 23 | 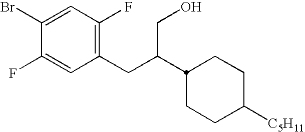 | 16h/55° C. NaH/THF 4h/120° C. NaH/ triglyme 4h/55° C. KH/THF |
| 24 | 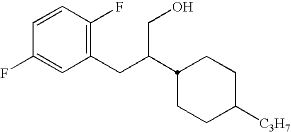 | 4h/50° C. 16h/60° C. |
| 25 | 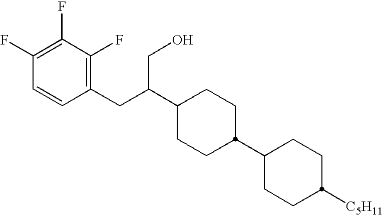 | 1h/40° C. 8h/60° C. |

| Ex- | | Yield |
|---|---|---|
| ample | Compound | [%] |
| 19 | 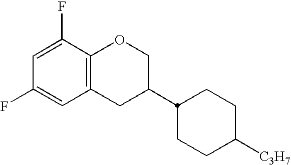 | 23 51 |
| 20 | 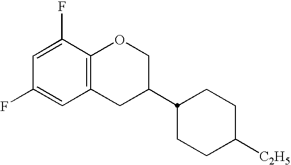 | 49 |
| 21 | 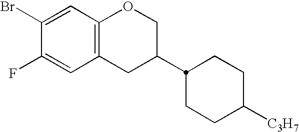 | 87 |
| 22 | 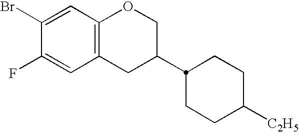 | 80 |
| 23 | 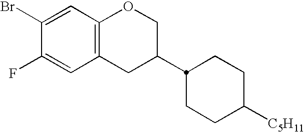 | 30 30 81 |
| 24 | 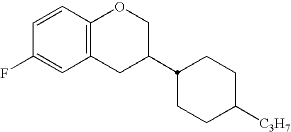 | 90 |
| 25 | 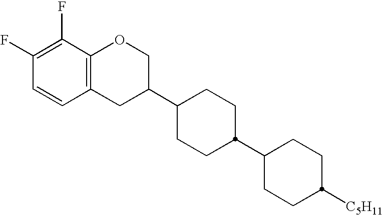 | 55 |

### EXAMPLE 26

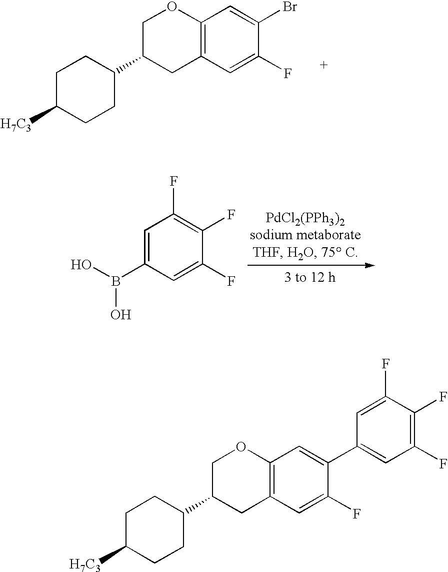

1.01 g of 3,4,5-trifluorophenylboronic acid (1.1 equiv.), 2.00 g of 6-fluoro-3-(4-propylcyclohexyl)-7-bromochromane (1.0 equiv.), 1.3 g of sodium metaborate octahydrate (0.84 equiv.) and 141 mg of bis(triphenylphosphine)palladium(II) chloride (3.5 mol %) are suspended in 20 ml of THF and 5 ml of water under nitrogen. The mixture is heated at 75° C. with vigorous stirring until the bromochromane has completely reacted (3 to 12 hours). After cooling, the aqueous phase is separated off and subsequently extracted three times with MTBE. The combined organic phases are washed with sodium chloride solution dried and evaporated in a rotary evaporator. The crude product is purified by column chromatography on silica gel using heptane/toluene (6:1) as eluent.

2.05 g of product having a content of 91%, corresponding to a yield of 82%, are obtained. Recrystallisation from heptane/isopropanol gives 1.4 g of product having a content of >99.5%.

C 102 N 115.1 I

Δn: 0.1397

Δ∈: 24.7

cl.p.: 88.5° C.

γ₁: 996 mPas

The following compounds of Examples 27 to 31 are obtained analogously to Example 26 using the corresponding precursors:

### EXAMPLE 27

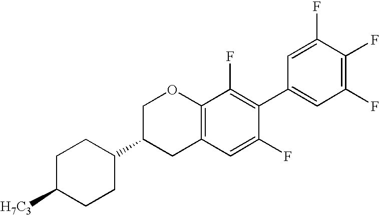

Yield: 68%

C 101 N (81, 9) I

Δn: 0.1305

Δ∈: 27.4

cl.p.: 76.3° C.

### EXAMPLE 28

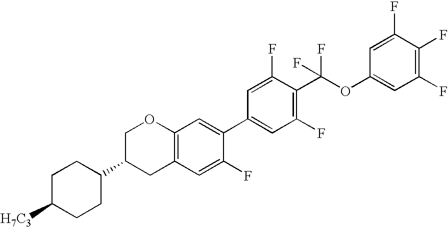

Yield: 86%

C 100 N 180.1 I

Δn: 0.1442

Δ∈: 35.1

cl.p.: 165.7° C.

γ₁: 1261 mPas

### EXAMPLE 29

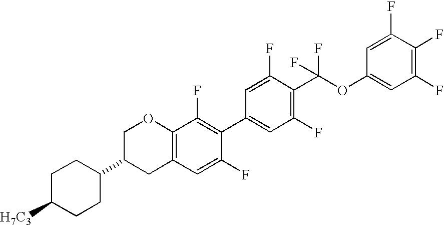

Yield: 72%

C 106 N 166.6 I

Δn: 0.1320

Δ∈: 36.2

cl.p.: 155.4° C.

### EXAMPLE 30

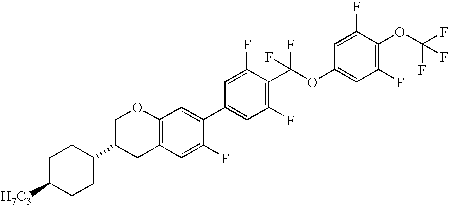

Yield: 88%

C 95 SmA 141 N 190 I

Δn: 0.1388

Δ∈: 35.8

cl.p.: 177.6° C.

### EXAMPLE 31

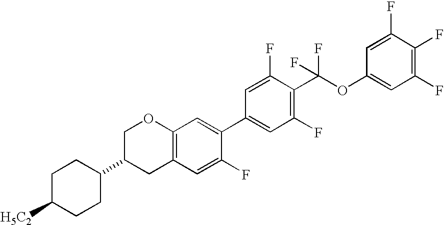

Yield: 71%

C 109 N 161.5 I

Δn: 0.1404

Δ∈: 34.8

cl.p.: 132.0° C.

The following compounds of Examples 32 to 2506 are obtained analogously to Examples 1 to 5 using the corresponding precursors:

Examples 32-106

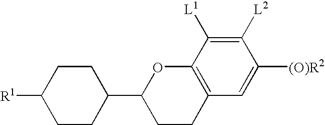

Examples 107-181

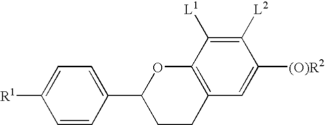

Examples 132-256

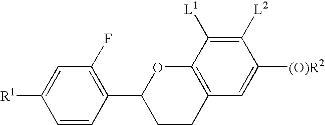

Examples 257-331

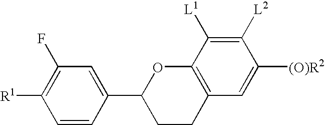

Examples 332-405

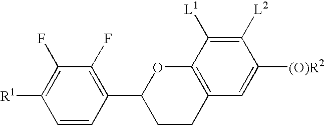

| Examples | | | | | R¹ | R² | L¹ | L² |
|---|---|---|---|---|---|---|---|---|
| 32, | 107, | 132, | 257, | 332, | CH₃ | CH₃ | H | F |
| 33, | 103, | 183, | 258, | 333, | CH₃ | CH₃ | F | H |
| 34, | 109, | 184, | 259, | 334, | CH₃ | CH₃ | F | F |
| 35, | 110, | 135, | 260, | 335, | CH₃ | C₂H₅ | H | F |
| 36, | 111, | 186, | 261, | 336, | CH₃ | C₂H₅ | F | H |
| 37, | 112, | 187, | 262, | 337, | CH₃ | C₂H₅ | F | F |
| 38, | 113, | 133, | 263, | 338, | CH₃ | C₃H₇ | H | F |
| 39, | 114, | 189, | 264, | 339, | CH₃ | C₃H₇ | F | H |
| 40, | 115, | 190, | 265, | 340, | CH₃ | C₃H₇ | F | F |
| 41, | 116, | 191, | 266, | 341, | CH₃ | C₄H₉ | H | F |
| 42, | 117, | 192, | 267, | 342, | CH₃ | C₄H₉ | F | H |
| 43, | 113, | 193, | 268, | 343, | CH₃ | C₄H₉ | F | F |
| 44, | 119, | 194, | 269, | 344, | CH₃ | C₅H₁₁ | H | F |
| 45, | 120, | 195, | 270, | 345, | CH₃ | C₅H₁₁ | F | H |
| 46, | 121, | 196, | 271, | 346, | CH₃ | C₅H₁₁ | F | F |
| 47, | 122, | 197, | 272, | 347, | C₂H₅ | CH₃ | H | F |
| 48, | 123, | 198, | 273, | 348, | C₂H₅ | CH₃ | F | H |
| 49, | 124, | 199, | 274, | 349, | C₂H₅ | CH₃ | F | F |
| 50, | 125, | 200, | 275, | 350, | C₂H₅ | C₂H₅ | H | F |
| 51, | 126, | 201, | 276, | 351, | C₂H₅ | C₂H₅ | F | H |
| 52, | 127, | 202, | 277, | 352, | C₂H₅ | C₂H₅ | F | F |
| 53, | 128, | 203, | 278, | 353, | C₂H₅ | C₃H₇ | H | F |
| 54, | 129, | 204, | 279, | 354, | C₂H₅ | C₃H₇ | F | H |
| 55, | 130, | 205, | 280, | 355, | C₂H₅ | C₃H₇ | F | F |
| 56, | 131, | 206, | 281, | 356, | C₂H₅ | C₄H₉ | H | F |
| 57, | 132, | 207, | 282, | 357, | C₂H₅ | C₄H₉ | F | H |
| 58, | 133, | 208, | 283, | 356, | C₂H₅ | C₄H₉ | F | F |
| 59, | 134, | 209, | 284, | 359, | C₂H₅ | C₅H₁₁ | H | F |
| 60, | 135, | 210, | 285, | 360, | C₂H₅ | C₅H₁₁ | F | H |
| 61, | 136, | 211, | 286, | 361, | C₂H₅ | C₅H₁₁ | F | F |
| 62, | 137, | 212, | 287, | 362, | C₃H₇ | CH₃ | H | F |
| 63, | 138, | 213, | 288, | 363, | C₃H₇ | CH₃ | F | H |
| 64, | 139, | 214, | 289, | 364, | C₃H₇ | CH₃ | F | F |
| 65, | 140, | 215, | 290, | 365, | C₃H₇ | C₂H₅ | H | F |
| 66, | 141, | 216, | 291, | 366, | C₃H₇ | C₂H₅ | F | H |
| 67, | 142, | 217, | 292, | 367, | C₃H₇ | C₂H₅ | F | F |
| 68, | 143, | 218, | 293, | 368, | C₃H₇ | C₃H₇ | H | F |
| 69, | 144, | 219, | 294, | 369, | C₃H₇ | C₃H₇ | F | H |
| 70, | 145, | 220, | 295, | 370, | C₃H₇ | C₃H₇ | F | F |
| 71, | 146, | 221, | 296, | 371, | C₃H₇ | C₄H₉ | H | F |
| 72, | 147, | 222, | 297, | 372, | C₃H₇ | C₄H₉ | F | H |
| 73, | 148, | 223, | 298, | 373, | C₃H₇ | C₄H₉ | F | F |
| 74, | 149, | 224, | 299, | 374, | C₃H₇ | C₅H₁₁ | H | F |
| 75, | 150, | 225, | 300, | 375, | C₃H₇ | C₅H₁₁ | F | H |
| 76, | 151, | 226, | 301, | 376, | C₃H₇ | C₅H₁₁ | F | F |
| 77, | 152, | 227, | 302, | 377, | C₄H₉ | CH₃ | H | F |
| 78, | 153, | 228, | 303, | 378, | C₄H₉ | CH₃ | F | H |
| 79, | 154, | 229, | 304, | 379, | C₄H₉ | CH₃ | F | F |
| 80, | 155, | 230, | 305, | 380, | C₄H₉ | C₂H₅ | H | F |
| 81, | 156, | 231, | 306, | 381, | C₄H₉ | C₂H₅ | F | H |
| 82, | 157, | 232, | 307, | 382, | C₄H₉ | C₂H₅ | F | F |
| 83, | 158, | 233, | 308, | 383, | C₄H₉ | C₃H₇ | H | F |
| 84, | 159, | 234, | 309, | 384, | C₄H₉ | C₃H₇ | F | H |
| 85, | 160, | 235, | 310, | 385, | C₄H₉ | C₃H₇ | F | F |
| 86, | 161, | 236, | 311, | 386, | C₄H₉ | C₄H₉ | H | F |
| 87, | 162, | 237, | 312, | 387, | C₄H₉ | C₄H₉ | F | H |
| 88, | 163, | 238, | 313, | 388, | C₄H₉ | C₄H₉ | F | F |
| 89, | 164, | 239, | 314, | 389, | C₄H₉ | C₅H₁₁ | H | F |
| 90, | 165, | 240, | 315, | 390, | C₄H₉ | C₅H₁₁ | F | H |
| 91, | 166, | 241, | 316, | 391, | C₄H₉ | C₅H₁₁ | F | F |
| 92, | 167, | 242, | 317, | 392, | C₅H₁₁ | CH₃ | H | F |
| 93, | 168, | 243, | 318, | 393, | C₅H₁₁ | CH₃ | F | H |
| 94, | 169, | 244, | 319, | 394, | C₅H₁₁ | CH₃ | F | F |
| 95, | 170, | 245, | 320, | 395, | C₅H₁₁ | C₂H₅ | H | F |
| 96, | 171, | 246, | 321, | 396, | C₅H₁₁ | C₂H₅ | F | H |
| 97, | 172, | 247, | 322, | 397, | C₅H₁₁ | C₂H₅ | F | F |
| 98, | 173, | 248, | 323, | 398, | C₅H₁₁ | C₃H₇ | H | F |
| 99, | 174, | 249, | 324, | 399, | C₅H₁₁ | C₃H₇ | F | H |
| 100, | 175, | 250, | 325, | 400, | C₅H₁₁ | C₃H₇ | F | F |
| 101, | 176, | 251, | 326, | 401, | C₅H₁₁ | C₄H₉ | H | F |
| 102, | 177, | 252, | 327, | 402, | C₅H₁₁ | C₄H₉ | F | H |
| 103, | 178, | 253, | 328, | 403, | C₅H₁₁ | C₄H₉ | F | F |
| 104, | 179, | 254, | 329, | 404, | C₅H₁₁ | C₅H₁₁ | H | F |
| 105, | 180, | 255, | 330, | 405, | C₅H₁₁ | C₅H₁₁ | F | H |
| 106, | 181, | 256, | 331, | 406, | C₅H₁₁ | C₅H₁₁ | F | F |

Examples 407-481

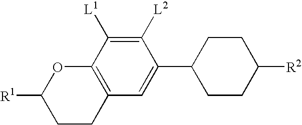

Examples 482-556

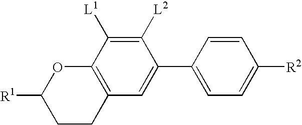

Examples 557-631

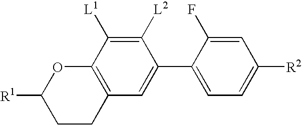

Examples 632-706

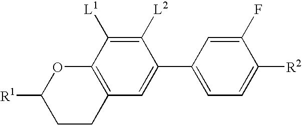

Examples 707-781

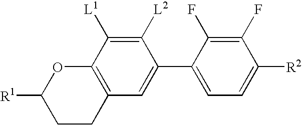

| Examples | | | | | R¹ | R² | L¹ | L² |
|---|---|---|---|---|---|---|---|---|
| 407, | 482, | 557, | 632, | 707, | CH₃ | CH₃ | H | F |
| 408, | 483, | 558, | 633, | 708, | CH₃ | CH₃ | F | H |
| 409, | 484, | 559, | 634, | 709, | CH₃ | CH₃ | F | F |
| 410, | 485, | 560, | 635, | 710, | CH₃ | C₂H₅ | H | F |
| 411, | 486, | 561, | 636, | 711, | CH₃ | C₂H₅ | F | H |
| 412, | 487, | 562, | 637, | 712, | CH₃ | C₂H₅ | F | F |
| 413, | 488, | 563, | 638, | 713, | CH₃ | C₃H₇ | H | F |
| 414, | 489, | 564, | 639, | 714, | CH₃ | C₃H₇ | F | H |
| 415, | 490, | 565, | 640, | 715, | CH₃ | C₃H₇ | F | F |
| 416, | 491, | 566, | 641, | 716, | CH₃ | C₄H₉ | H | F |
| 417, | 492, | 567, | 642, | 717, | CH₃ | C₄H₉ | F | H |
| 418, | 493, | 568, | 643, | 718, | CH₃ | C₄H₉ | F | F |
| 419, | 494, | 569, | 644, | 719, | CH₃ | C₅H₁₁ | H | F |
| 420, | 495, | 570, | 645, | 720, | CH₃ | C₅H₁₁ | F | H |
| 421, | 496, | 571, | 646, | 721, | CH₃ | C₅H₁₁ | F | F |
| 422, | 497, | 572, | 647, | 722, | C₂H₅ | CH₃ | H | F |
| 423, | 498, | 573, | 648, | 723, | C₂H₅ | CH₃ | F | H |
| 424, | 499, | 574, | 649, | 724, | C₂H₅ | CH₃ | F | F |
| 425, | 500, | 575, | 650, | 725, | C₂H₅ | C₂H₅ | H | F |
| 426, | 501, | 576, | 651, | 726, | C₂H₅ | C₂H₅ | F | H |
| 427, | 502, | 577, | 652, | 727, | C₂H₅ | C₂H₅ | F | F |
| 428, | 503, | 578, | 653, | 728, | C₂H₅ | C₃H₇ | H | F |
| 429, | 504, | 579, | 654, | 729, | C₂H₅ | C₃H₇ | F | H |
| 430, | 505, | 580, | 655, | 730, | C₂H₅ | C₃H₇ | F | F |
| 431, | 506, | 581, | 656, | 731, | C₂H₅ | C₄H₉ | H | F |
| 432, | 507, | 582, | 657, | 732, | C₂H₅ | C₄H₉ | F | H |
| 433, | 508, | 583, | 658, | 733, | C₂H₅ | C₄H₉ | F | F |
| 434, | 509, | 584, | 659, | 734, | C₂H₅ | C₅H₁₁ | H | F |
| 435, | 510, | 585, | 660, | 735, | C₂H₅ | C₅H₁₁ | F | H |
| 436, | 511, | 586, | 661, | 736, | C₂H₅ | C₅H₁₁ | F | F |
| 437, | 512, | 587, | 662, | 737, | C₃H₇ | CH₃ | H | F |
| 438, | 513, | 588, | 663, | 738, | C₃H₇ | CH₃ | F | H |
| 439, | 514, | 589, | 664, | 739, | C₃H₇ | CH₃ | F | F |
| 440, | 515, | 590, | 665, | 740, | C₃H₇ | C₂H₅ | H | F |
| 441, | 516, | 591, | 666, | 741, | C₃H₇ | C₂H₅ | F | H |
| 442, | 517, | 592, | 667, | 742, | C₃H₇ | C₂H₅ | F | F |
| 443, | 518, | 593, | 668, | 743, | C₃H₇ | C₃H₇ | H | F |
| 444, | 519, | 594, | 669, | 744, | C₃H₇ | C₃H₇ | F | H |
| 445, | 520, | 595, | 670, | 745, | C₃H₇ | C₃H₇ | F | F |
| 446, | 521, | 596, | 671, | 746, | C₃H₇ | C₄H₉ | H | F |
| 447, | 522, | 597, | 672, | 747, | C₃H₇ | C₄H₉ | F | H |
| 448, | 523, | 598, | 673, | 748, | C₃H₇ | C₄H₉ | F | F |
| 449, | 524, | 599, | 674, | 749, | C₃H₇ | C₅H₁₁ | H | F |
| 450, | 525, | 600, | 675, | 750, | C₃H₇ | C₅H₁₁ | F | H |
| 451, | 526, | 601, | 676, | 751, | C₃H₇ | C₅H₁₁ | F | F |
| 452, | 527, | 602, | 677, | 752, | C₄H₉ | CH₃ | H | F |
| 453, | 528, | 603, | 678, | 753, | C₄H₉ | CH₃ | F | H |
| 454, | 529, | 604, | 679, | 754, | C₄H₉ | CH₃ | F | F |
| 455, | 530, | 605, | 680, | 755, | C₄H₉ | C₂H₅ | H | F |
| 456, | 531, | 606, | 681, | 756, | C₄H₉ | C₂H₅ | F | H |
| 457, | 532, | 607, | 682, | 757, | C₄H₉ | C₂H₅ | F | F |
| 458, | 533, | 608, | 683, | 758, | C₄H₉ | C₃H₇ | H | F |
| 459, | 534, | 609, | 684, | 759, | C₄H₉ | C₃H₇ | F | H |
| 460, | 535, | 610, | 685, | 760, | C₄H₉ | C₃H₇ | F | F |
| 461, | 536, | 611, | 686, | 761, | C₄H₉ | C₄H₉ | H | F |
| 462, | 537, | 612, | 687, | 762, | C₄H₉ | C₄H₉ | F | H |
| 463, | 538, | 613, | 688, | 763, | C₄H₉ | C₄H₉ | F | F |
| 464, | 539, | 614, | 689, | 764, | C₄H₉ | C₅H₁₁ | H | F |
| 465, | 540, | 615, | 690, | 765, | C₄H₉ | C₅H₁₁ | F | H |
| 466, | 541, | 616, | 691, | 766, | C₄H₉ | C₅H₁₁ | F | F |
| 467, | 542, | 617, | 692, | 767, | C₅H₁₁ | CH₃ | H | F |
| 468, | 543, | 618, | 693, | 768, | C₅H₁₁ | CH₃ | F | H |
| 469, | 544, | 619, | 694, | 769, | C₅H₁₁ | CH₃ | F | F |
| 470, | 545, | 620, | 695, | 770, | C₅H₁₁ | C₂H₅ | H | F |
| 471, | 546, | 621, | 696, | 771, | C₅H₁₁ | C₂H₅ | F | H |
| 472, | 547, | 622, | 697, | 772, | C₅H₁₁ | C₂H₅ | F | F |
| 473, | 548, | 623, | 698, | 773, | C₅H₁₁ | C₃H₇ | H | F |
| 474, | 549, | 624, | 699, | 774, | C₅H₁₁ | C₃H₇ | F | H |
| 475, | 550, | 625, | 700, | 775, | C₅H₁₁ | C₃H₇ | F | F |
| 476, | 551, | 626, | 701, | 776, | C₅H₁₁ | C₄H₉ | H | F |
| 477, | 552, | 627, | 702, | 777, | C₅H₁₁ | C₄H₉ | F | H |
| 478, | 553, | 628, | 703, | 778, | C₅H₁₁ | C₄H₉ | F | F |
| 479, | 554, | 629, | 704, | 779, | C₅H₁₁ | C₅H₁₁ | H | F |
| 480, | 555, | 630, | 705, | 780, | C₅H₁₁ | C₅H₁₁ | F | H |
| 481, | 556, | 631, | 706, | 781, | C₅H₁₁ | C₅H₁₁ | F | F |

Examples 752-856

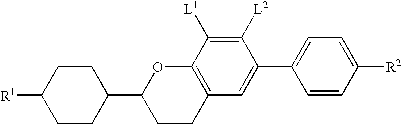

Examples 857-931

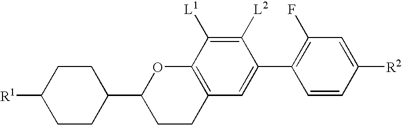

Examples 932-1006

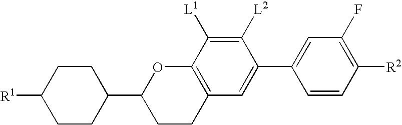

Examples 1007-1081

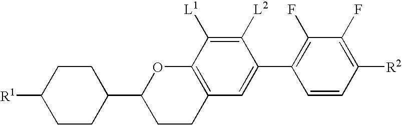

| Examples | | | | R¹ | R² | L¹ | L² |
|---|---|---|---|---|---|---|---|
| 782, | 857, | 932, | 1007, | CH₃ | CH₃ | H | F |
| 783, | 855, | 933, | 1008, | CH₃ | CH₃ | F | H |
| 784, | 859, | 934, | 1009, | CH₃ | CH₃ | F | F |
| 785, | 860, | 935, | 1010, | CH₃ | C₂H₅ | H | F |
| 786, | 861, | 936, | 1011, | CH₃ | C₂H₅ | F | H |
| 787, | 862, | 937, | 1012, | CH₃ | C₂H₅ | F | F |
| 788, | 863, | 938, | 1013, | CH₃ | C₃H₇ | H | F |
| 789, | 864, | 939, | 1014, | CH₃ | C₃H₇ | F | H |
| 790, | 865, | 940, | 1015, | CH₃ | C₃H₇ | F | F |
| 791, | 866, | 941, | 1016, | CH₃ | C₄H₉ | H | F |
| 792, | 867, | 942, | 1017, | CH₃ | C₄H₉ | F | H |
| 793, | 868, | 943, | 1018, | CH₃ | C₄H₉ | F | F |
| 794, | 869, | 944, | 1019, | CH₃ | C₅H₁₁ | H | F |
| 795, | 870, | 945, | 1020, | CH₃ | C₅H₁₁ | F | H |
| 796, | 871, | 946, | 1021, | CH₃ | C₅H₁₁ | F | F |
| 797, | 872, | 947, | 1022, | C₂H₅ | CH₃ | H | F |
| 798, | 873, | 948, | 1023, | C₂H₅ | CH₃ | F | H |
| 799, | 874, | 949, | 1024, | C₂H₅ | CH₃ | F | F |
| 800, | 875, | 950, | 1025, | C₂H₅ | C₂H₅ | H | F |
| 801, | 876, | 951, | 1026, | C₂H₅ | C₂H₅ | F | H |
| 802, | 877, | 952, | 1027, | C₂H₅ | C₂H₅ | F | F |
| 803, | 878, | 953, | 1028, | C₂H₅ | C₃H₇ | H | F |
| 804, | 879, | 954, | 1029, | C₂H₅ | C₃H₇ | F | H |
| 805, | 880, | 955, | 1030, | C₂H₅ | C₃H₇ | F | F |
| 806, | 881, | 956, | 1031, | C₂H₅ | C₄H₉ | H | F |
| 807, | 882, | 957, | 1032, | C₂H₅ | C₄H₉ | F | H |
| 808, | 883, | 958, | 1033, | C₂H₅ | C₄H₉ | F | F |
| 809, | 884, | 959, | 1034, | C₂H₅ | C₅H₁₁ | H | F |
| 810, | 885, | 960, | 1035, | C₂H₅ | C₅H₁₁ | F | H |
| 811, | 886, | 961, | 1036, | C₂H₅ | C₅H₁₁ | F | F |
| 812, | 887, | 962, | 1037, | C₃H₇ | CH₃ | H | F |
| 813, | 888, | 963, | 1038, | C₃H₇ | CH₃ | F | H |
| 814, | 889, | 964, | 1039, | C₃H₇ | CH₃ | F | F |
| 815, | 890, | 965, | 1040, | C₃H₇ | C₂H₅ | H | F |
| 816, | 891, | 966, | 1041, | C₃H₇ | C₂H₅ | F | H |
| 817, | 892, | 967, | 1042, | C₃H₇ | C₂H₅ | F | F |
| 818, | 893, | 968, | 1043, | C₃H₇ | C₃H₇ | H | F |
| 819, | 894, | 969, | 1044, | C₃H₇ | C₃H₇ | F | H |
| 820, | 895, | 970, | 1045, | C₃H₇ | C₃H₇ | F | F |
| 821, | 896, | 971, | 1046, | C₃H₇ | C₄H₉ | H | F |
| 822, | 897, | 972, | 1047, | C₃H₇ | C₄H₉ | F | H |
| 823, | 898, | 973, | 1048, | C₃H₇ | C₄H₉ | F | F |
| 824, | 899, | 974, | 1049, | C₃H₇ | C₅H₁₁ | H | F |
| 825, | 900, | 975, | 1050, | C₃H₇ | C₅H₁₁ | F | H |
| 826, | 901, | 976, | 1051, | C₃H₇ | C₅H₁₁ | F | F |
| 827, | 902, | 977, | 1052, | C₄H₉ | CH₃ | H | F |
| 828, | 903, | 978, | 1053, | C₄H₉ | CH₃ | F | H |
| 829, | 904, | 979, | 1054, | C₄H₉ | CH₃ | F | F |
| 830, | 905, | 980, | 1055, | C₄H₉ | C₂H₅ | H | F |
| 831, | 906, | 981, | 1056, | C₄H₉ | C₂H₅ | F | H |
| 832, | 907, | 982, | 1057, | C₄H₉ | C₂H₅ | F | F |
| 833, | 908, | 983, | 1058, | C₄H₉ | C₃H₇ | H | F |
| 834, | 909, | 984, | 1059, | C₄H₉ | C₃H₇ | F | H |
| 835, | 910, | 985, | 1060, | C₄H₉ | C₃H₇ | F | F |
| 836, | 911, | 986, | 1061, | C₄H₉ | C₄H₉ | H | F |
| 837, | 912, | 987, | 1062, | C₄H₉ | C₄H₉ | F | H |
| 838, | 913, | 988, | 1063, | C₄H₉ | C₄H₉ | F | F |
| 839, | 914, | 989, | 1064, | C₄H₉ | C₅H₁₁ | H | F |
| 840, | 916, | 990, | 1065, | C₄H₉ | C₅H₁₁ | F | H |
| 841, | 916, | 991, | 1066, | C₄H₉ | C₅H₁₁ | F | F |
| 842, | 917, | 992, | 1067, | C₅H₁₁ | CH₃ | H | F |
| 843, | 918, | 993, | 1068, | C₅H₁₁ | CH₃ | F | H |
| 844, | 919, | 994, | 1069, | C₅H₁₁ | CH₃ | F | F |
| 845, | 920, | 995, | 1070, | C₅H₁₁ | C₂H₅ | H | F |
| 846, | 921, | 996, | 1071, | C₅H₁₁ | C₂H₅ | F | H |
| 847, | 922, | 997, | 1072, | C₅H₁₁ | C₂H₅ | F | F |
| 848, | 923, | 998, | 1073, | C₅H₁₁ | C₃H₇ | H | F |
| 849, | 924, | 999, | 1074, | C₅H₁₁ | C₃H₇ | F | H |
| 850, | 925, | 1000, | 1075, | C₅H₁₁ | C₃H₇ | F | F |
| 851, | 926, | 1001, | 1076, | C₅H₁₁ | C₄H₉ | H | F |
| 852, | 927, | 1002, | 1077, | C₅H₁₁ | C₄H₉ | F | H |
| 853, | 928, | 1003, | 1078, | C₅H₁₁ | C₄H₉ | F | F |
| 854, | 929, | 1004, | 1079, | C₅H₁₁ | C₅H₁₁ | H | F |
| 855, | 930, | 1005, | 1080, | C₅H₁₁ | C₅H₁₁ | F | H |
| 856, | 931, | 1006, | 1081, | C₅H₁₁ | C₅H₁₁ | F | F |

Examples 1082-1156

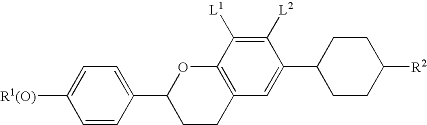

Examples 1157-1231

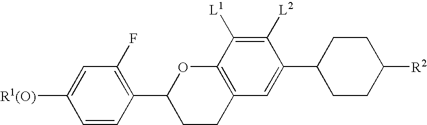

Examples 1232-1306

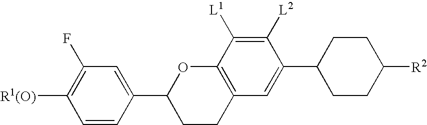

Examples 1307-1381

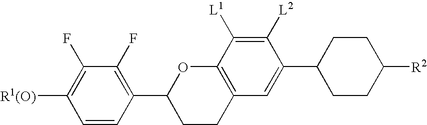

| Examples | | | | R¹ | R² | L¹ | L² |
|---|---|---|---|---|---|---|---|
| 1082, | 1157, | 1232, | 1307, | CH₃ | CH₃ | H | F |
| 1083, | 1158, | 1233, | 1308, | CH₃ | CH₃ | F | H |
| 1084, | 1159, | 1234, | 1309, | CH₃ | CH₃ | F | F |
| 1085, | 1160, | 1235, | 1310, | CH₃ | C₂H₅ | H | F |
| 1086, | 1161, | 1236, | 1311, | CH₃ | C₂H₅ | F | H |
| 1087, | 1162, | 1237, | 1312, | CH₃ | C₂H₅ | F | F |
| 1088, | 1163, | 1238, | 1313, | CH₃ | C₃H₇ | H | F |
| 1089, | 1164, | 1239, | 1314, | CH₃ | C₃H₇ | F | H |
| 1090, | 1165, | 1240, | 1315, | CH₃ | C₃H₇ | F | F |
| 1091, | 1166, | 1241, | 1316, | CH₃ | C₄H₉ | H | F |
| 1092, | 1167, | 1242, | 1317, | CH₃ | C₄H₉ | F | H |
| 1093, | 1168, | 1243, | 1318, | CH₃ | C₄H₉ | F | F |
| 1094, | 1169, | 1244, | 1319, | CH₃ | C₅H₁₁ | H | F |
| 1095, | 1170, | 1245, | 1320, | CH₃ | C₅H₁₁ | F | H |
| 1096, | 1171, | 1246, | 1321, | CH₃ | C₅H₁₁ | F | F |
| 1097, | 1172, | 1247, | 1322, | C₂H₅ | CH₃ | H | F |
| 1098, | 1173, | 1248, | 1323, | C₂H₅ | CH₃ | F | H |
| 1099, | 1174, | 1249, | 1324, | C₂H₅ | CH₃ | F | F |
| 1100, | 1175, | 1250, | 1325, | C₂H₅ | C₂H₅ | H | F |
| 1101, | 1176, | 1251, | 1326, | C₂H₅ | C₂H₅ | F | H |
| 1102, | 1177, | 1252, | 1327, | C₂H₅ | C₂H₅ | F | F |
| 1103, | 1178, | 1253, | 1328, | C₂H₅ | C₃H₇ | H | F |
| 1104, | 1179, | 1254, | 1329, | C₂H₅ | C₃H₇ | F | H |
| 1105, | 1180, | 1255, | 1330, | C₂H₅ | C₃H₇ | F | F |
| 1106, | 1181, | 1256, | 1331, | C₂H₅ | C₄H₉ | H | F |
| 1107, | 1182, | 1257, | 1332, | C₂H₅ | C₄H₉ | F | H |
| 1108, | 1183, | 1258, | 1333, | C₂H₅ | C₄H₉ | F | F |
| 1109, | 1184, | 1259, | 1334, | C₂H₅ | C₅H₁₁ | H | F |
| 1110, | 1185, | 1260, | 1335, | C₂H₅ | C₅H₁₁ | F | H |
| 1111, | 1186, | 1261, | 1336, | C₂H₅ | C₅H₁₁ | F | F |
| 1112, | 1187, | 1262, | 1337, | C₃H₇ | CH₃ | H | F |
| 1113, | 1188, | 1263, | 1338, | C₃H₇ | CH₃ | F | H |
| 1114, | 1189, | 1264, | 1339, | C₃H₇ | CH₃ | F | F |
| 1115, | 1190, | 1265, | 1340, | C₃H₇ | C₂H₅ | H | F |
| 1116, | 1191, | 1266, | 1341, | C₃H₇ | C₂H₅ | F | H |
| 1117, | 1192, | 1267, | 1342, | C₃H₇ | C₂H₅ | F | F |
| 1118, | 1193, | 1268, | 1343, | C₃H₇ | C₃H₇ | H | F |
| 1119, | 1194, | 1269, | 1344, | C₃H₇ | C₃H₇ | F | H |
| 1120, | 1195, | 1270, | 1345, | C₃H₇ | C₃H₇ | F | F |
| 1121, | 1196, | 1271, | 1346, | C₃H₇ | C₄H₉ | H | F |
| 1122, | 1197, | 1272, | 1347, | C₃H₇ | C₄H₉ | F | H |
| 1123, | 1198, | 1273, | 1348, | C₃H₇ | C₄H₉ | F | F |
| 1124, | 1199, | 1274, | 1349, | C₃H₇ | C₅H₁₁ | H | F |
| 1125, | 1200, | 1275, | 1350, | C₃H₇ | C₅H₁₁ | F | H |
| 1126, | 1201, | 1276, | 1351, | C₃H₇ | C₅H₁₁ | F | F |
| 1127, | 1202, | 1277, | 1352, | C₄H₉ | CH₃ | H | F |
| 1128, | 1203, | 1278, | 1353, | C₄H₉ | CH₃ | F | H |
| 1129, | 1204, | 1279, | 1354, | C₄H₉ | CH₃ | F | F |
| 1130, | 1205, | 1280, | 1355, | C₄H₉ | C₂H₅ | H | F |
| 1131, | 1206, | 1281, | 1356, | C₄H₉ | C₂H₅ | F | H |
| 1132, | 1207, | 1262, | 1357, | C₄H₉ | C₂H₅ | F | F |
| 1133, | 1208, | 1283, | 1358, | C₄H₉ | C₃H₇ | H | F |
| 1134, | 1209, | 1284, | 1359, | C₄H₉ | C₃H₇ | F | H |
| 1135, | 1210, | 1285, | 1360, | C₄H₉ | C₃H₇ | F | F |
| 1136, | 1211, | 1286, | 1361, | C₄H₉ | C₄H₉ | H | F |
| 1137, | 1212, | 1287, | 1362, | C₄H₉ | C₄H₉ | F | H |
| 1138, | 1213, | 1288, | 1363, | C₄H₉ | C₄H₉ | F | F |
| 1139, | 1214, | 1289, | 1364, | C₄H₉ | C₅H₁₁ | H | F |
| 1140, | 1215, | 1290, | 1365, | C₄H₉ | C₅H₁₁ | F | H |
| 1141, | 1216, | 1291, | 1366, | C₄H₉ | C₅H₁₁ | F | F |
| 1142, | 1217, | 1292, | 1367, | C₅H₁₁ | CH₃ | H | F |
| 1143, | 1218, | 1293, | 1368, | C₅H₁₁ | CH₃ | F | H |
| 1144, | 1219, | 1294, | 1369, | C₅H₁₁ | CH₃ | F | F |
| 1145, | 1220, | 1295, | 1370, | C₅H₁₁ | C₂H₅ | H | F |
| 1146, | 1221, | 1296, | 1371, | C₅H₁₁ | C₂H₅ | F | H |
| 1147, | 1222, | 1297, | 1372, | C₅H₁₁ | C₂H₅ | F | F |
| 1148, | 1223, | 1298, | 1373, | C₅H₁₁ | C₃H₇ | H | F |
| 1149, | 1224, | 1299, | 1374, | C₅H₁₁ | C₃H₇ | F | H |
| 1150, | 1225, | 1300, | 1375, | C₅H₁₁ | C₃H₇ | F | F |
| 1151, | 1226, | 1301, | 1376, | C₅H₁₁ | C₄H₉ | H | F |
| 1152, | 1227, | 1302, | 1377, | C₅H₁₁ | C₄H₉ | F | H |
| 1153, | 1228, | 1303, | 1378, | C₅H₁₁ | C₄H₉ | F | F |
| 1154, | 1229, | 1304, | 1379, | C₅H₁₁ | C₅H₁₁ | H | F |
| 1155, | 1230, | 1305, | 1380, | C₅H₁₁ | C₅H₁₁ | F | H |
| 1156, | 1231, | 1306, | 1381, | C₅H₁₁ | C₅H₁₁ | F | F |

Examples 1382-1456

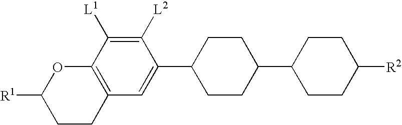

Examples 1457-1531

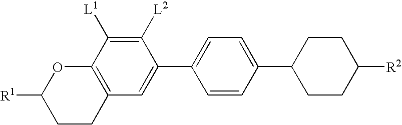

Examples 1532-1606

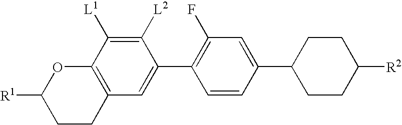

Examples 1607-1681

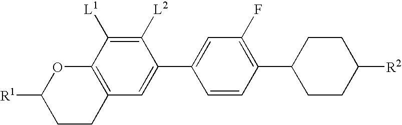

Examples 1682-1756

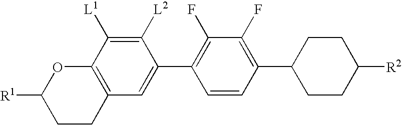

| Examples | | | | | R¹ | R² | L¹ | L² |
|---|---|---|---|---|---|---|---|---|
| 1382, | 1457, | 1532, | 1607, | 1682, | CH₃ | CH₃ | H | F |
| 1383, | 1458, | 1533, | 1608, | 1683, | CH₃ | CH₃ | F | H |
| 1384, | 1459, | 1534, | 1609, | 1684, | CH₃ | CH₃ | F | F |
| 1385, | 1460, | 1535, | 1610, | 1685, | CH₃ | C₂H₅ | H | F |
| 1386, | 1461, | 1536; | 1611, | 1686, | CH₃ | C₂H₅ | F | H |
| 1387, | 1462, | 1537, | 1612, | 1687, | CH₃ | C₂H₅ | F | F |
| 1388, | 1463, | 1538, | 1613, | 1688, | CH₃ | C₃H₇ | H | F |
| 1389, | 1464, | 1539, | 1614, | 1689, | CH₃ | C₃H₇ | F | H |
| 1390, | 1465, | 1540, | 1615, | 1690, | CH₃ | C₃H₇ | F | F |
| 1391, | 1466, | 1541, | 1616, | 1691, | CH₃ | C₄H₉ | H | F |
| 1392, | 1467, | 1542, | 1617, | 1692, | CH₃ | C₄H₉ | F | H |
| 1393, | 1468, | 1543, | 1618, | 1693, | CH₃ | C₄H₉ | F | F |
| 1394, | 1469, | 1544, | 1619, | 1694, | CH₃ | C₅H₁₁ | H | F |
| 1395, | 1470, | 1545, | 1620, | 1695, | CH₃ | C₅H₁₁ | F | H |
| 1396, | 1471, | 1546, | 1621, | 1696, | CH₃ | C₅H₁₁ | F | F |
| 1397, | 1472, | 1547, | 1622, | 1697, | C₂H₅ | CH₃ | H | F |
| 1398, | 1473, | 1548, | 1623, | 1698, | C₂H₅ | CH₃ | F | H |
| 1399, | 1474, | 1549, | 1624, | 1699, | C₂H₅ | CH₃ | F | F |
| 1400, | 1475, | 1550, | 1625, | 1700, | C₂H₅ | C₂H₅ | H | F |
| 1401, | 1476, | 1551, | 1626, | 1701, | C₂H₅ | C₂H₅ | F | H |
| 1402, | 1477, | 1552, | 1627, | 1702, | C₂H₅ | C₂H₅ | F | F |
| 1403, | 1478, | 1553, | 1628, | 1703, | C₂H₅ | C₃H₇ | H | F |
| 1404, | 1479, | 1554, | 1629, | 1704, | C₂H₅ | C₃H₇ | F | H |
| 1405, | 1480, | 1555, | 1630, | 1705, | C₂H₅ | C₃H₇ | F | F |
| 1406, | 1481, | 1556, | 1631, | 1706, | C₂H₅ | C₄H₉ | H | F |
| 1407, | 1482, | 1557, | 1632, | 1707, | C₂H₅ | C₄H₉ | F | H |
| 1408, | 1483, | 1558, | 1633, | 1708, | C₂H₅ | C₄H₉ | F | F |
| 1409, | 1484, | 1559, | 1634, | 1709, | C₂H₅ | C₅H₁₁ | H | F |
| 1410, | 1485, | 1560, | 1635, | 1710, | C₂H₅ | C₅H₁₁ | F | H |
| 1411, | 1486, | 1561, | 1636, | 1711, | C₂H₅ | C₅H₁₁ | F | F |
| 1412, | 1487, | 1562, | 1637, | 1712, | C₃H₇ | CH₃ | H | F |
| 1413, | 1488, | 1563, | 1638, | 1713, | C₃H₇ | CH₃ | F | H |
| 1414, | 1489, | 1564, | 1639, | 1714, | C₃H₇ | CH₃ | F | F |
| 1415, | 1490, | 1565, | 1640, | 1715, | C₃H₇ | C₂H₅ | H | F |
| 1416, | 1491, | 1566, | 1641, | 1716, | C₃H₇ | C₂H₅ | F | H |
| 1417, | 1492, | 1567, | 1642, | 1717, | C₃H₇ | C₂H₅ | F | F |
| 1418, | 1493, | 1568, | 1643, | 1718, | C₃H₇ | C₃H₇ | H | F |
| 1419, | 1494, | 1569, | 1644, | 1719, | C₃H₇ | C₃H₇ | F | H |
| 1420, | 1495, | 1570, | 1645, | 1720, | C₃H₇ | C₃H₇ | F | F |
| 1421, | 1496, | 1571, | 1646, | 1721, | C₃H₇ | C₄H₉ | H | F |
| 1422, | 1497, | 1572, | 1647, | 1722, | C₃H₇ | C₄H₉ | F | H |
| 1423, | 1498, | 1573, | 1648, | 1723, | C₃H₇ | C₄H₉ | F | F |
| 1424, | 1499, | 1574, | 1649, | 1724, | C₃H₇ | C₅H₁₁ | H | F |
| 1425, | 1500, | 1575, | 1650, | 1725, | C₃H₇ | C₅H₁₁ | F | H |
| 1426, | 1501, | 1576, | 1651, | 1726, | C₃H₇ | C₅H₁₁ | F | F |
| 1427, | 1502, | 1577, | 1652, | 1727, | C₄H₉ | CH₃ | H | F |
| 1428, | 1503, | 1578, | 1653, | 1728, | C₄H₉ | CH₃ | F | H |
| 1429, | 1504, | 1579, | 1654, | 1729, | C₄H₉ | CH₃ | F | F |
| 1430, | 1505, | 1580, | 1655, | 1730, | C₄H₉ | C₂H₅ | H | F |
| 1431, | 1506, | 1581, | 1656, | 1731, | C₄H₉ | C₂H₅ | F | H |
| 1432, | 1507, | 1582, | 1657, | 1732, | C₄H₉ | C₂H₅ | F | F |
| 1433, | 1508, | 1583, | 1658, | 1733, | C₄H₉ | C₃H₇ | H | F |
| 1434, | 1509, | 1584, | 1659, | 1734, | C₄H₉ | C₃H₇ | F | H |
| 1435, | 1510, | 1585, | 1660, | 1735, | C₄H₉ | C₃H₇ | F | F |
| 1436, | 1511, | 1586, | 1661, | 1736, | C₄H₉ | C₄H₉ | H | F |
| 1437, | 1512, | 1587, | 1662, | 1737, | C₄H₉ | C₄H₉ | F | H |
| 1438, | 1513, | 1588, | 1663, | 1738, | C₄H₉ | C₄H₉ | F | F |
| 1439, | 1514, | 1589, | 1664, | 1739, | C₄H₉ | C₅H₁₁ | H | F |
| 1440, | 1515, | 1590, | 1665, | 1740, | C₄H₉ | C₅H₁₁ | F | H |
| 1441, | 1516, | 1591, | 1666, | 1741, | C₄H₉ | C₅H₁₁ | F | F |
| 1442, | 1517, | 1592, | 1667, | 1742, | C₅H₁₁ | CH₃ | H | F |
| 1443, | 1518, | 1593, | 1668, | 1743, | C₅H₁₁ | CH₃ | F | H |
| 1444, | 1619, | 1594, | 1669, | 1744, | C₅H₁₁ | CH₃ | F | F |
| 1445, | 1520, | 1595, | 1670, | 1745, | C₅H₁₁ | C₂H₅ | H | F |
| 1446, | 1521, | 1596, | 1671, | 1746, | C₅H₁₁ | C₂H₅ | F | H |
| 1447, | 1522, | 1597, | 1672, | 1747, | C₅H₁₁ | C₂H₅ | F | F |
| 1448, | 1523, | 1598, | 1673, | 1748, | C₅H₁₁ | C₃H₇ | H | F |
| 1449, | 1524, | 1599, | 1674, | 1749, | C₅H₁₁ | C₃H₇ | F | H |
| 1450, | 1525, | 1600, | 1675, | 1750, | C₅H₁₁ | C₃H₇ | F | F |
| 1451, | 1526, | 1601, | 1676, | 1751, | C₅H₁₁ | C₄H₉ | H | F |
| 1452, | 1527, | 1602, | 1677, | 1752, | C₅H₁₁ | C₄H₉ | F | H |
| 1453, | 1528, | 1603, | 1678, | 1753, | C₅H₁₁ | C₄H₉ | F | F |
| 1454, | 1529, | 1604, | 1679, | 1754, | C₅H₁₁ | C₅H₁₁ | H | F |
| 1455, | 1530, | 1605, | 1680, | 1755, | C₅H₁₁ | C₅H₁₁ | F | H |
| 1456, | 1531, | 1606, | 1681, | 1756, | C₅H₁₁ | C₅H₁₁ | F | F |

| Examples | R¹ | R² | L¹ | L² |
|---|---|---|---|---|
| 1757, 1832, 1907, 1982, 2057, | CH₃ | CH₃ | H | F |
| 1758, 1833, 1908, 1983, 2058, | CH₃ | CH₃ | F | H |
| 1759, 1834, 1909, 1984, 2059, | CH₃ | CH₃ | F | F |
| 1760, 1835, 1910, 1985, 2060, | CH₃ | C₂H₅ | H | F |
| 1761, 1836, 1911, 1986, 2061, | CH₃ | C₂H₅ | F | H |
| 1762, 1837, 1912, 1987, 2062, | CH₃ | C₂H₅ | F | F |
| 1763, 1838, 1913, 1988, 2063, | CH₃ | C₃H₇ | H | F |
| 1764, 1839, 1914, 1989, 2064, | CH₃ | C₃H₇ | F | H |
| 1765, 1840, 1915, 1990, 2065, | CH₃ | C₃H₇ | F | F |
| 1766, 1841, 1916, 1991, 2066, | CH₃ | C₄H₉ | H | F |
| 1767, 1842, 1917, 1992, 2067, | CH₃ | C₄H₉ | F | H |
| 1768, 1843, 1918, 1993, 2068, | CH₃ | C₄H₉ | F | F |
| 1769, 1844, 1919, 1994, 2069, | CH₃ | C₅H₁₁ | H | F |
| 1770, 1845, 1920, 1995, 2070, | CH₃ | C₅H₁₁ | F | H |
| 1771, 1846, 1921, 1996, 2071, | CH₃ | C₅H₁₁ | F | F |
| 1772, 1847, 1922, 1997, 2072, | C₂H₅ | CH₃ | H | F |
| 1773, 1848, 1923, 1998, 2073, | C₂H₅ | CH₃ | F | H |
| 1774, 1849, 1924, 1999, 2074, | C₂H₅ | CH₃ | F | F |
| 1775, 1850, 1925, 2000, 2075, | C₂H₅ | C₂H₅ | H | F |
| 1776, 1851, 1926, 2001, 2076, | C₂H₅ | C₂H₅ | F | H |
| 1777, 1852, 1927, 2002, 2077, | C₂H₅ | C₂H₅ | F | F |
| 1778, 1853, 1928, 2003, 2078, | C₂H₅ | C₃H₇ | H | F |
| 1779, 1854, 1929, 2004, 2079, | C₂H₅ | C₃H₇ | F | H |
| 1780, 1855, 1930, 2005, 2080, | C₂H₅ | C₃H₇ | F | F |
| 1781, 1856, 1931, 2006, 2081, | C₂H₅ | C₄H₉ | H | F |
| 1782, 1857, 1932, 2007, 2082, | C₂H₅ | C₄H₉ | F | H |
| 1783, 1858, 1933, 2008, 2083, | C₂H₅ | C₄H₉ | F | F |
| 1784, 1859, 1934, 2009, 2084, | C₂H₅ | C₅H₁₁ | H | F |
| 1785, 1860, 1935, 2010, 2085, | C₂H₅ | C₅H₁₁ | F | H |
| 1786, 1861, 1936, 2011, 2086, | C₂H₅ | C₅H₁₁ | F | F |
| 1787, 1862, 1937, 2012, 2087, | C₃H₇ | CH₃ | H | F |
| 1788, 1863, 1938, 2013, 2088, | C₃H₇ | CH₃ | F | H |
| 1789, 1864, 1939, 2014, 2089, | C₃H₇ | CH₃ | F | F |
| 1790, 1865, 1940, 2015, 2090, | C₃H₇ | C₂H₅ | H | F |
| 1791, 1866, 1941, 2016, 2091, | C₃H₇ | C₂H₅ | F | H |
| 1792, 1867, 1942, 2017, 2092, | C₃H₇ | C₂H₅ | F | F |
| 1793, 1868, 1943, 2018, 2093, | C₃H₇ | C₃H₇ | H | F |
| 1794, 1869, 1944, 2019, 2094, | C₃H₇ | C₃H₇ | F | H |
| 1795, 1870, 1945, 2020, 2095, | C₃H₇ | C₃H₇ | F | F |
| 1796, 1871, 1946, 2021, 2096, | C₃H₇ | C₄H₉ | H | F |
| 1797, 1872, 1947, 2022, 2097, | C₃H₇ | C₄H₉ | F | H |
| 1798, 1873, 1948, 2023, 2098, | C₃H₇ | C₄H₉ | F | F |
| 1799, 1874, 1949, 2024, 2099, | C₃H₇ | C₅H₁₁ | H | F |
| 1800, 1875, 1950, 2025, 2100, | C₃H₇ | C₅H₁₁ | F | H |
| 1801, 1876, 1951, 2026, 2101, | C₃H₇ | C₅H₁₁ | F | F |
| 1802, 1877, 1952, 2027, 2102, | C₄H₉ | CH₃ | H | F |
| 1803, 1878, 1953, 2028, 2103, | C₄H₉ | CH₃ | F | H |
| 1804, 1879, 1954, 2029, 2104, | C₄H₉ | CH₃ | F | F |
| 1805, 1880, 1955, 2030, 2105, | C₄H₉ | C₂H₅ | H | F |
| 1806, 1881, 1956, 2031, 2106, | C₄H₉ | C₂H₅ | F | H |
| 1807, 1882, 1957, 2032, 2107, | C₄H₉ | C₂H₅ | F | F |
| 1808, 1883, 1958, 2033, 2108, | C₄H₉ | C₃H₇ | H | F |
| 1809, 1884, 1959, 2034, 2109, | C₄H₉ | C₃H₇ | F | H |
| 1810, 1885, 1960, 2035, 2110, | C₄H₉ | C₃H₇ | F | F |
| 1811, 1886, 1961, 2036, 2111, | C₄H₉ | C₄H₉ | H | F |
| 1812, 1887, 1962, 2037, 2112, | C₄H₉ | C₄H₉ | F | H |
| 1813, 1888, 1963, 2038, 2113, | C₄H₉ | C₄H₉ | F | F |
| 1814, 1889, 1964, 2039, 2114, | C₄H₉ | C₅H₁₁ | H | F |
| 1815, 1890, 1965, 2040, 2115, | C₄H₉ | C₅H₁₁ | F | H |
| 1816, 1891, 1966, 2041, 2116, | C₄H₉ | C₅H₁₁ | F | F |
| 1817, 1892, 1967, 2042, 2117, | C₅H₁₁ | CH₃ | H | F |
| 1818, 1893, 1968, 2043, 2118, | C₅H₁₁ | CH₃ | F | H |
| 1819, 1894, 1969, 2044, 2119, | C₅H₁₁ | CH₃ | F | F |
| 1820, 1895, 1970, 2045, 2120, | C₅H₁₁ | C₂H₅ | H | F |
| 1821, 1896, 1971, 2046, 2121, | C₅H₁₁ | C₂H₅ | F | H |
| 1822, 1897, 1972, 2047, 2122, | C₅H₁₁ | C₂H₅ | F | F |
| 1823, 1898, 1973, 2048, 2123, | C₅H₁₁ | C₃H₇ | H | F |
| 1824, 1899, 1974, 2049, 2124, | C₅H₁₁ | C₃H₇ | F | H |
| 1825, 1900, 1975, 2050, 2125, | C₅H₁₁ | C₃H₇ | F | F |
| 1826, 1901, 1976, 2051, 2126, | C₅H₁₁ | C₄H₉ | H | F |
| 1827, 1902, 1977, 2052, 2127, | C₅H₁₁ | C₄H₉ | F | H |
| 1828, 1903, 1978, 2053, 2128, | C₅H₁₁ | C₄H₉ | F | F |
| 1829, 1904, 1979, 2054, 2129, | C₅H₁₁ | C₅H₁₁ | H | F |
| 1830, 1905, 1980, 2055, 2130, | C₅H₁₁ | C₅H₁₁ | F | H |
| 1831, 1906, 1981, 2056, 2131, | C₅H₁₁ | C₅H₁₁ | F | F |

Examples 1757-1831

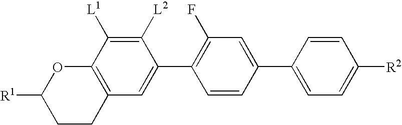

Examples 1832-1906

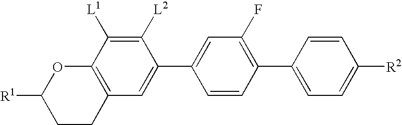

Examples 1907-1981

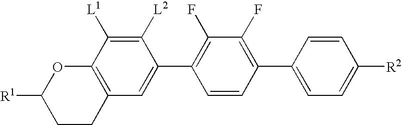

Examples 1982-2056

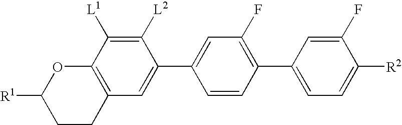

Examples 2057-2131

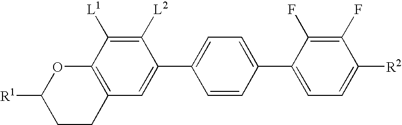

| Examples | R¹ | R² | L¹ | L² |
|---|---|---|---|---|
| 2132, 2207, 2282, 2357, 2432, | CH₃ | CH₃ | H | F |
| 2133, 2208, 2283, 2358, 2433, | CH₃ | CH₃ | F | H |
| 2134, 2209, 2284, 2359, 2434, | CH₃ | CH₃ | F | F |
| 2135, 2210, 2285, 2360, 2435, | CH₃ | C₂H₅ | H | F |
| 2136, 2211, 2286, 2361, 2436, | CH₃ | C₂H₅ | F | H |
| 2137, 2212, 2287, 2362, 2437, | CH₃ | C₂H₅ | F | F |
| 2138, 2213, 2288, 2363, 2433, | CH₃ | C₃H₇ | H | F |
| 2139, 2214, 2289, 2364, 2439, | CH₃ | C₃H₇ | F | H |
| 2140, 2215, 2290, 2365, 2440, | CH₃ | C₃H₇ | F | F |
| 2141, 2216, 2291, 2366, 2441, | CH₃ | C₄H₉ | H | F |
| 2142, 2217, 2292, 2367, 2442, | CH₃ | C₄H₉ | F | H |
| 2143, 2218, 2293, 2368, 2443, | CH₃ | C₄H₉ | F | F |
| 2144, 2219, 2294, 2369, 2444, | CH₃ | C₅H₁₁ | H | F |
| 2145, 2220, 2295, 2370, 2445, | CH₃ | C₅H₁₁ | F | H |
| 2146, 2221, 2296, 2371, 2446, | CH₃ | C₅H₁₁ | F | F |
| 2147, 2222, 2297, 2372, 2447, | C₂H₅ | CH₃ | H | F |
| 2148, 2223, 2298, 2373, 2448, | C₂H₅ | CH₃ | F | H |
| 2149, 2224, 2299, 2374, 2449, | C₂H₅ | CH₃ | F | F |
| 2150, 2225, 2300, 2375, 2450, | C₂H₅ | C₂H₅ | H | F |
| 2151, 2226, 2301, 2376, 2451, | C₂H₅ | C₂H₅ | F | H |
| 2152, 2227, 2302, 2377, 2452, | C₂H₅ | C₂H₅ | F | F |
| 2153, 2228, 2303, 2378, 2453, | C₂H₅ | C₃H₇ | H | F |
| 2154, 2229, 2304, 2379, 2454, | C₂H₅ | C₃H₇ | F | H |
| 2155, 2230, 2305, 2380, 2455, | C₂H₅ | C₃H₇ | F | F |
| 2156, 2231, 2306, 2381, 2456, | C₂H₅ | C₄H₉ | H | F |
| 2157, 2232, 2307, 2382, 2457, | C₂H₅ | C₄H₉ | F | H |
| 2158, 2233, 2308, 2383, 2458, | C₂H₅ | C₄H₉ | F | F |
| 2159, 2234, 2309, 2384, 2459, | C₂H₅ | C₅H₁₁ | H | F |
| 2160, 2235, 2310, 2385, 2460, | C₂H₅ | C₅H₁₁ | F | H |
| 2161, 2236, 2311, 2386, 2461, | C₂H₅ | C₅H₁₁ | F | F |
| 2162, 2237, 2312, 2387, 2462, | C₃H₇ | CH₃ | H | F |
| 2163, 2238, 2313, 2388, 2463, | C₃H₇ | CH₃ | F | H |
| 2164, 2239, 2314, 2389, 2464, | C₃H₇ | CH₃ | F | F |
| 2165, 2240, 2315, 2390, 2465, | C₃H₇ | C₂H₅ | H | F |
| 2166, 2241, 2316, 2391, 2466, | C₃H₇ | C₂H₅ | F | H |
| 2167, 2242, 2317, 2392, 2467, | C₃H₇ | C₂H₅ | F | F |
| 2168, 2243, 2318, 2393, 2468, | C₃H₇ | C₃H₇ | H | F |
| 2169, 2244, 2319, 2394, 2469, | C₃H₇ | C₃H₇ | F | H |
| 2170, 2245, 2320, 2395, 2470, | C₃H₇ | C₃H₇ | F | F |
| 2171, 2246, 2321, 2396, 2471, | C₃H₇ | C₄H₉ | H | F |
| 2172, 2247, 2322, 2397, 2472, | C₃H₇ | C₄H₉ | F | H |
| 2173, 2248, 2323, 2398, 2473, | C₃H₇ | C₄H₉ | F | F |
| 2174, 2249, 2324, 2399, 2474, | C₃H₇ | C₅H₁₁ | H | F |
| 2175, 2250, 2325, 2400, 2475, | C₃H₇ | C₅H₁₁ | F | H |
| 2176, 2251, 2326, 2401, 2476, | C₃H₇ | C₅H₁₁ | F | F |
| 2177, 2252, 2327, 2402, 2477, | C₄H₉ | CH₃ | H | F |
| 2178, 2253, 2328, 2403, 2478, | C₄H₉ | CH₃ | F | H |
| 2179, 2254, 2329, 2404, 2479, | C₄H₉ | CH₃ | F | F |
| 2180, 2255, 2330, 2405, 2480, | C₄H₉ | C₂H₅ | H | F |
| 2181, 2256, 2331, 2406, 2481, | C₄H₉ | C₂H₅ | F | H |
| 2182, 2257, 2332, 2407, 2482, | C₄H₉ | C₂H₅ | F | F |
| 2183, 2258, 2333, 2408, 2483, | C₄H₉ | C₃H₇ | H | F |
| 2184, 2259, 2334, 2409, 2484, | C₄H₉ | C₃H₇ | F | H |
| 2185, 2260, 2335, 2410, 2485, | C₄H₉ | C₃H₇ | F | F |
| 2186, 2261, 2336, 2411, 2486, | C₄H₉ | C₄H₉ | H | F |
| 2187, 2262, 2337, 2412, 2487, | C₄H₉ | C₄H₉ | F | H |
| 2188, 2263, 2338, 2413, 2488, | C₄H₉ | C₄H₉ | F | F |
| 2189, 2264, 2339, 2414, 2489, | C₄H₉ | C₅H₁₁ | H | F |
| 2190, 2265, 2340, 2415, 2490, | C₄H₉ | C₅H₁₁ | F | H |
| 2191, 2266, 2341, 2416, 2491, | C₄H₉ | C₅H₁₁ | F | F |
| 2192, 2267, 2342, 2417, 2492, | C₅H₁₁ | CH₃ | H | F |
| 2193, 2268, 2343, 2418, 2493, | C₅H₁₁ | CH₃ | F | H |
| 2194, 2269, 2344, 2419, 2494, | C₅H₁₁ | CH₃ | F | F |
| 2195, 2270, 2345, 2420, 2495, | C₅H₁₁ | C₂H₅ | H | F |
| 2196, 2271, 2346, 2421, 2496, | C₅H₁₁ | C₂H₅ | F | H |
| 2197, 2272, 2347, 2422, 2497, | C₅H₁₁ | C₂H₅ | F | F |
| 2198, 2273, 2348, 2423, 2498, | C₅H₁₁ | C₃H₇ | H | F |
| 2199, 2274, 2349, 2424, 2499, | C₅H₁₁ | C₃H₇ | F | H |
| 2200, 2275, 2350, 2425, 2500, | C₅H₁₁ | C₃H₇ | F | F |
| 2201, 2276, 2351, 2426, 2501, | C₅H₁₁ | C₄H₉ | H | F |
| 2202, 2277, 2352, 2427, 2502, | C₅H₁₁ | C₄H₉ | F | H |
| 2203, 2278, 2353, 2428, 2503, | C₅H₁₁ | C₄H₉ | F | F |
| 2204, 2279, 2354, 2429, 2504, | C₅H₁₁ | C₅H₁₁ | H | F |
| 2205, 2280, 2355, 2430, 2505, | C₅H₁₁ | C₅H₁₁ | F | H |
| 2206, 2281, 2356, 2431, 2506, | C₅H₁₁ | C₅H₁₁ | F | F |

Examples 2132-2206

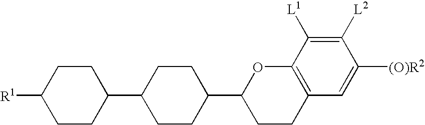

Examples 2207-2281

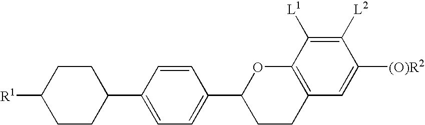

Examples 2282-2356

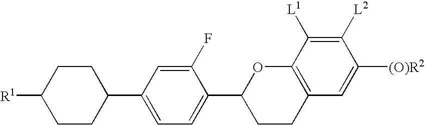

Examples 2357-2431

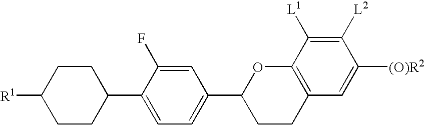

Examples 2432-2506

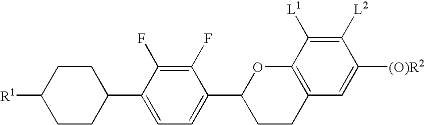

The following compounds of Examples 2507 to 4306 are obtained analogously to Examples 6 to 31 using the corresponding precursors:

| Examples | L¹ | L² | L³ | L⁴ | R² |
|---|---|---|---|---|---|
| 2507, 2552, 2597, 2642, | H | H | H | H | F |
| 2508, 2553, 2598, 2643, | H | H | H | F | F |
| 2509, 2554, 2599, 2644, | H | F | H | H | F |
| 2510, 2555, 2600, 2645, | F | F | H | H | F |
| 2511, 2556, 2601, 2646, | H | H | F | F | F |
| 2512, 2557, 2602, 2647, | H | F | H | F | F |
| 2513, 2558, 2603, 2648, | H | F | F | F | F |
| 2514, 2559, 2604, 2649, | F | F | H | F | F |
| 2515, 2560, 2605, 2650, | F | F | F | F | F |
| 2516, 2561, 2606, 2651, | H | H | H | H | CF₃ |
| 2517, 2562, 2607, 2652, | H | H | H | F | CF₃ |
| 2518, 2563, 2608, 2653, | H | F | H | H | CF₃ |
| 2519, 2564, 2609, 2654, | F | F | H | H | CF₃ |
| 2520, 2565, 2610, 2655, | H | H | F | F | CF₃ |
| 2521, 2566, 2611, 2656, | H | F | H | F | CF₃ |
| 2522, 2567, 2612, 2657, | H | F | F | F | CF₃ |
| 2523, 2568, 2613, 2658, | F | F | H | F | CF₃ |
| 2524, 2569, 2614, 2659, | F | F | F | F | CF₃ |
| 2525, 2570, 2615, 2660, | H | H | H | H | OCF₃ |
| 2526, 2571, 2616, 2661, | H | H | H | F | OCF₃ |
| 2527, 2572, 2617, 2662, | H | F | H | H | OCF₃ |
| 2528, 2573, 2618, 2663, | F | F | H | H | OCF₃ |
| 2529, 2574, 2619, 2664, | H | H | F | F | OCF₃ |
| 2530, 2575, 2620, 2665, | H | F | H | F | OCF₃ |
| 2531, 2576, 2621, 2666, | H | F | F | F | OCF₃ |
| 2532, 2577, 2622, 2667, | F | F | H | F | OCF₃ |
| 2533, 2578, 2623, 2668, | F | F | F | F | OCF₃ |
| 2534, 2579, 2624, 2669, | H | H | H | H | Cl |
| 2535, 2580, 2625, 2670, | H | H | H | F | Cl |
| 2536, 2581, 2626, 2671, | H | F | H | H | Cl |
| 2537, 2582, 2627, 2672, | F | F | H | H | Cl |
| 2538, 2583, 2628, 2673, | H | H | F | F | Cl |
| 2539, 2584, 2629, 2674, | H | F | H | F | Cl |
| 2540, 2585, 2630, 2675, | H | F | F | F | Cl |
| 2541, 2586, 2631, 2676, | F | F | H | F | Cl |
| 2542, 2587, 2632, 2677, | F | F | F | F | Cl |
| 2543, 2588, 2633, 2678, | H | H | H | H | CN |
| 2544, 2589, 2634, 2679, | H | H | H | F | CN |
| 2545, 2590, 2635, 2680, | H | F | H | H | CN |
| 2546, 2591, 2636, 2681, | F | F | H | H | CN |
| 2547, 2592, 2637, 2682, | H | H | F | F | CN |
| 2548, 2593, 2638, 2683, | H | F | H | F | CN |
| 2549, 2594, 2639, 2684, | H | F | F | F | CN |
| 2550, 2595, 2640, 2685, | F | F | H | F | CN |
| 2551, 2596, 2641, 2686, | F | F | F | F | CN |

where ak(en)yl is selected from: CH₃, C₂H₅, C₃H₇, C₄H₉, C₅H₁₁,
CH═CH₂, CH═CH—CH₃ and CH═CH—C₃H₇.
Examples 2507 to 2551

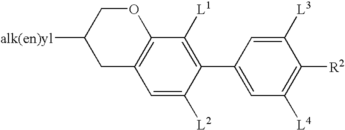

Examples 2552 to 2596

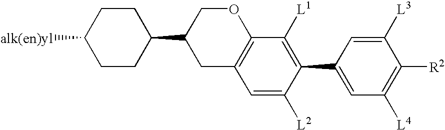

Examples 2597 to 2641

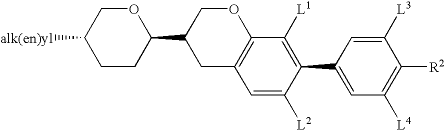

Examples 2642 to 2686

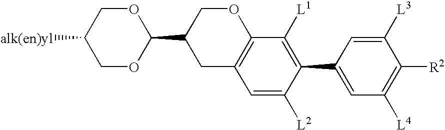

| Examples | L¹ | L² | L³ | L⁴ | R² |
|---|---|---|---|---|---|
| 2687, 2732, 2777, | H | H | H | H | F |
| 2688, 2733, 2778, | H | H | H | F | F |
| 2689, 2734, 2779, | H | F | H | H | F |
| 2690, 2735, 2780, | F | F | H | H | F |
| 2691, 2736, 2781, | H | H | F | F | F |
| 2692, 2737, 2782, | H | F | H | F | F |
| 2693, 2738, 2783, | H | F | F | F | F |
| 2694, 2739, 2784, | F | F | H | F | F |
| 2695, 2740, 2785, | F | F | F | F | F |
| 2696, 2741, 2786, | H | H | H | H | CF₃ |
| 2697, 2742, 2787, | H | H | H | F | CF₃ |
| 2698, 2743, 2788, | H | F | H | H | CF₃ |
| 2699, 2744, 2789, | F | F | H | H | CF₃ |
| 2700, 2745, 2790, | H | H | F | F | CF₃ |
| 2701, 2746, 2791, | H | F | H | F | CF₃ |
| 2702, 2747, 2792, | H | F | F | F | CF₃ |
| 2703, 2748, 2793, | F | F | H | F | CF₃ |
| 2704, 2749, 2794, | F | F | F | F | CF₃ |
| 2705, 2750, 2795, | H | H | H | H | OCF₃ |
| 2706, 2751, 2796, | H | H | H | F | OCF₃ |
| 2707, 2752, 2797, | H | F | H | H | OCF₃ |
| 2708, 2753, 2798, | F | F | H | H | OCF₃ |
| 2709, 2754, 2799, | H | H | F | F | OCF₃ |
| 2710, 2755, 2800, | H | F | H | F | OCF₃ |
| 2711, 2756, 2801, | H | F | F | F | OCF₃ |
| 2712, 2757, 2802, | F | F | H | F | OCF₃ |
| 2713, 2758, 2803, | F | F | F | F | OCF₃ |
| 2714, 2759, 2804, | H | H | H | H | Cl |
| 2715, 2760, 2805, | H | H | H | F | Cl |
| 2716, 2761, 2806; | H | F | H | H | Cl |
| 2717, 2762, 2807, | F | F | H | H | Cl |
| 2718, 2763, 2808, | H | H | F | F | Cl |
| 2719, 2764, 2809, | H | F | H | F | Cl |
| 2720, 2765, 2810, | H | F | F | F | Cl |
| 2721, 2766, 2811, | F | F | H | F | Cl |
| 2722, 2767, 2812, | F | F | F | F | Cl |
| 2723, 2768, 2813, | H | H | H | H | CN |
| 2724, 2769, 2814, | H | H | H | F | CN |
| 2725, 2770, 2815, | H | F | H | H | CN |
| 2726, 2771, 2816, | F | F | H | H | CN |
| 2727, 2772, 2317, | H | H | F | F | CN |
| 2728, 2773, 2818, | H | F | H | F | CN |
| 2729, 2774, 2819, | H | F | F | F | CN |
| 2730, 2775, 2820, | F | F | H | F | CN |
| 2731, 2776, 2821, | F | F | F | F | CN |

where alk(en)yl is selected from: CH₃, C₂H₅, C₃H₇,
C₄H₉, C₅H₁₁, CH═CH₂, CH═CH—CH₃ and CH═C₃H₇.
Examples 2687 to 2731

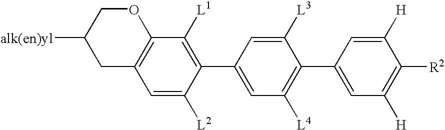

Examples 2732 to 2776

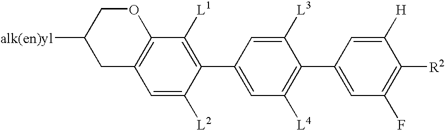

Examples 2777 to 2821

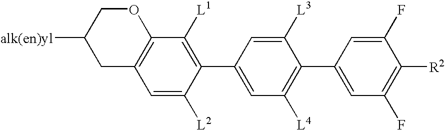

| Examples | L¹ | L² | L³ | L⁴ | R² |
|---|---|---|---|---|---|
| 2822, 2867, 2912, | H | H | H | H | F |
| 2823, 2868, 2913, | H | H | H | F | F |
| 2824, 2869, 2914, | H | F | H | H | F |
| 2825, 2870, 2915, | F | F | H | H | F |
| 2826, 2871, 2916, | H | H | F | F | F |
| 2827, 2872, 2917, | H | F | H | F | F |
| 2828, 2873, 2918, | H | F | F | F | F |
| 2829, 2874, 2919, | F | F | H | F | F |
| 2830, 2875, 2920, | F | F | F | F | F |
| 2831, 2876, 2921, | H | H | H | H | CF₃ |
| 2832, 2877, 2922, | H | H | H | F | CF₃ |
| 2833, 2878, 2923, | H | F | H | H | CF₃ |
| 2834, 2879, 2924, | F | F | H | H | CF₃ |
| 2835, 2880, 2925, | H | H | F | F | CF₃ |
| 2836, 2881, 2926, | H | F | H | F | CF₃ |
| 2837, 2882, 2927, | H | F | F | F | CF₃ |
| 2838, 2883, 2928, | F | F | H | F | CF₃ |
| 2839, 2884, 2929, | F | F | F | F | CF₃ |
| 2840, 2885, 2930, | H | H | H | H | OCF₃ |
| 2841, 2886, 2931, | H | H | H | F | OCF₃ |
| 2842, 2887, 2932, | H | F | H | H | OCF₃ |
| 2843, 2888, 2933, | F | F | H | H | OCF₃ |
| 2844, 2889, 2934, | H | H | F | F | OCF₃ |
| 2845, 2890, 2935, | H | F | H | F | OCF₃ |
| 2846, 2891, 2936, | H | F | F | F | OCF₃ |
| 2847, 2892, 2937, | F | F | H | F | OCF₃ |
| 2848, 2893, 2938, | F | F | F | F | OCF₃ |
| 2849, 2894, 2939, | H | H | H | H | Cl |
| 2850, 2895, 2940, | H | H | H | F | Cl |
| 2851, 2896, 2941, | H | F | H | H | Cl |
| 2852, 2897, 2942, | F | F | H | H | Cl |
| 2853, 2898, 2943, | H | H | F | F | Cl |
| 2854, 2899, 2944, | H | F | H | F | Cl |
| 2855, 2900, 2945, | H | F | F | F | Cl |
| 2856, 2901, 2946, | F | F | H | F | Cl |
| 2857, 2902, 2947, | F | F | F | F | Cl |
| 2858, 2903, 2948, | H | H | H | H | CN |
| 2859, 2904, 2949, | H | H | H | F | CN |
| 2860, 2905, 2950, | H | F | H | H | CN |
| 2861, 2906, 2951, | F | F | H | H | CN |
| 2862, 2907, 2952, | H | H | F | F | CN |
| 2863, 2908, 2953, | H | F | H | F | CN |
| 2864, 2909, 2954, | H | F | F | F | CN |
| 2865, 2910, 2955, | F | F | H | F | CN |
| 2866, 2911, 2956, | F | F | F | F | CN |

where alk(en)yl is selected from: CH₃, C₂H₅, C₃H₇,
C₄H₉, C₅H₁₁, CH═CH₂, CH═CH—CH₃ and CH═C₃H₇.
Examples 2822 to 2866

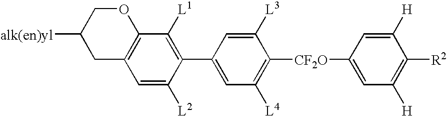

Examples 2867 to 2911

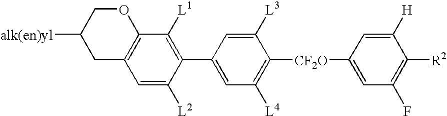

Examples 2912 to 2956

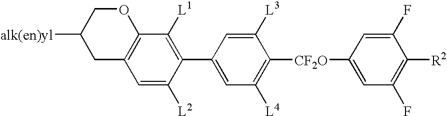

| Examples | L¹ | L² | L³ | L⁴ | R² |
|---|---|---|---|---|---|
| 2957, 3002, 3047, | H | H | H | H | F |
| 2958, 3003, 3048, | H | H | H | F | F |
| 2959, 3004, 3049, | H | F | H | H | F |
| 2960, 3005, 3050, | F | F | H | H | F |
| 2961, 3006, 3051, | H | H | F | F | F |
| 2962, 3007, 3052, | H | F | H | F | F |
| 2963, 3008, 3053, | H | F | F | F | F |
| 2964, 3009, 3054, | F | F | H | F | F |
| 2965, 3010, 3055, | F | F | F | F | F |
| 2966, 3011, 3056, | H | H | H | H | CF₃ |
| 2967, 3012, 3057, | H | H | H | F | CF₃ |
| 2968, 3013, 3058, | H | F | H | H | CF₃ |
| 2969, 3014, 3059, | F | F | H | H | CF₃ |
| 2970, 3015, 3060, | H | H | F | F | CF₃ |
| 2971, 3016, 3061, | H | F | H | F | CF₃ |
| 2972, 3017, 3062, | H | F | F | F | CF₃ |
| 2973, 3018, 3063, | F | F | H | F | CF₃ |
| 2974, 3019, 3064, | F | F | F | F | CF₃ |
| 2975, 3020, 3065, | H | H | H | H | OCF₃ |
| 2976, 3021, 3066, | H | H | H | F | OCF₃ |
| 2977, 3022, 3067, | H | F | H | H | OCF₃ |
| 2978, 3023, 3068, | F | F | H | H | OCF₃ |
| 2979, 3024, 3069, | H | H | F | F | OCF₃ |
| 2980, 3025, 3070, | H | F | H | F | OCF₃ |
| 2981, 3026, 3071, | H | F | F | F | OCF₃ |
| 2982, 3027, 3072, | F | F | H | F | OCF₃ |
| 2983, 3028, 3073, | F | F | F | F | OCF₃ |
| 2984, 3029, 3074, | H | H | H | H | Cl |
| 2985, 3030, 3075, | H | H | H | F | Cl |
| 2986, 3031, 3076, | H | F | H | H | Cl |
| 2987, 3032, 3077, | F | F | H | H | Cl |
| 2988, 3033, 3078, | H | H | F | F | Cl |
| 2989, 3034, 3079, | H | F | H | F | Cl |
| 2990, 3035, 3080, | H | F | F | F | Cl |
| 2991, 3036, 3081, | F | F | H | F | Cl |
| 2992, 3037, 3082, | F | F | F | F | Cl |
| 2993, 3038, 3083, | H | H | H | H | CN |
| 2994, 3039, 3084, | H | H | H | F | CN |
| 2995, 3040, 3085, | H | F | H | H | CN |
| 2996, 3041, 3086, | F | F | H | H | CN |
| 2997, 3042, 3087, | H | H | F | F | CN |
| 2998, 3043, 3088, | H | F | H | F | CN |
| 2999, 3044, 3089, | H | F | F | F | CN |
| 3000, 3045, 3090, | F | F | H | F | CN |
| 3001, 3046, 3091, | F | F | F | F | CN |

where alk(en)yl is selected from: CH₃, C₂H₅, C₃H₇,
C₄H₉, C₅H₁₁, CH═CH₂, CH═CH—CH₃ and CH═C₃H₇.
Examples 2957 to 3001

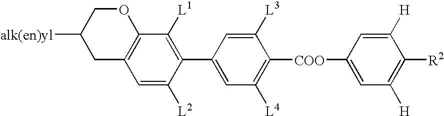

Examples 3002 to 3046

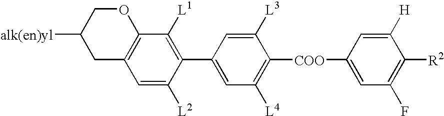

Examples 3047 to 3091

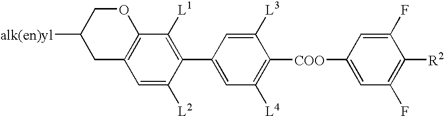

| Examples | L¹ | L² | L³ | L⁴ | R² |
|---|---|---|---|---|---|
| 3092, 3137, 3182, | H | H | H | H | F |
| 3093, 3138, 3183, | H | H | H | F | F |
| 3094, 3139, 3184, | H | F | H | H | F |
| 3095, 3140, 3185, | F | F | H | H | F |
| 3096, 3141, 3186, | H | H | F | F | F |
| 3097, 3142, 3187, | H | F | H | F | F |
| 3098, 3143, 3188, | H | F | F | F | F |
| 3099, 3144, 3189, | F | F | H | F | F |
| 3100, 3145, 3190, | F | F | F | F | F |
| 3101, 3146, 3191, | H | H | H | H | CF₃ |
| 3102, 3147, 3192, | H | H | H | F | CF₃ |
| 3103, 3148, 3193, | H | F | H | H | CF₃ |
| 3104, 3149, 3194, | F | F | H | H | CF₃ |
| 3105, 3150, 3195, | H | H | F | F | CF₃ |
| 3106, 3151, 3196, | H | F | H | F | CF₃ |
| 3107, 3152, 3197, | H | F | F | F | CF₃ |
| 3108, 3153, 3198, | F | F | H | F | CF₃ |
| 3109, 3154, 3199, | F | F | F | F | CF₃ |
| 3110, 3155, 3200, | H | H | H | H | OCF₃ |
| 3111, 3156, 3201, | H | H | H | F | OCF₃ |
| 3112, 3157, 3202, | H | F | H | H | OCF₃ |
| 3113, 3158, 3203, | F | F | H | H | OCF₃ |
| 3114, 3159, 3204, | H | H | F | F | OCF₃ |
| 3115, 3160, 3205, | H | F | H | F | OCF₃ |
| 3116, 3161, 3206, | H | F | F | F | OCF₃ |
| 3117, 3162, 3207, | F | F | H | F | OCF₃ |
| 3118, 3163, 3208, | F | F | F | F | OCF₃ |
| 3119, 3164, 3209, | H | H | H | H | Cl |
| 3120, 3165, 3210, | H | H | H | F | Cl |
| 3121, 3166, 3211, | H | F | H | H | Cl |
| 3122, 3167, 3212, | F | F | H | H | Cl |
| 3123, 3168, 3213, | H | H | F | F | Cl |
| 3124, 3169, 3214, | H | F | H | F | Cl |
| 3125, 3170, 3215, | H | F | F | F | Cl |
| 3126, 3171, 3216, | F | F | H | F | Cl |
| 3127, 3172, 3217, | F | F | F | F | Cl |
| 3128, 3173, 3218, | H | H | H | H | CN |
| 3129, 3174, 3219, | H | H | H | F | CN |
| 3130, 3175, 3220, | H | F | H | H | CN |
| 3131, 3176, 3221, | F | F | H | H | CN |
| 3132, 3177, 3222, | H | H | F | F | CN |
| 3133, 3178, 3223, | H | F | H | F | CN |
| 3134, 3179, 3224, | H | F | F | F | CN |
| 3135, 3180, 3225, | F | F | H | F | CN |
| 3136, 3181, 3226, | F | F | F | F | CN |

where alk(en)yl is selected from: CH₃, C₂H₅, C₃H₇,
C₄H₉, C₅H₁₁, CH═CH₂, CH═CH—CH₃ and CH═C₃H₇.
Examples 3092 to 3136

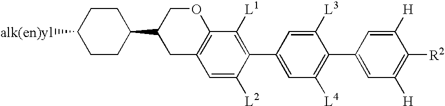

Examples 3137 to 3181

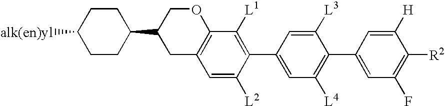

Examples 3182 to 3226

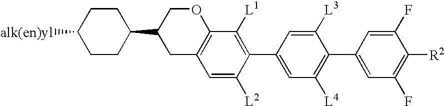

| Examples | L¹ | L² | L³ | L⁴ | R² |
|---|---|---|---|---|---|
| 3227, 3272, 3317, | H | H | H | H | F |
| 3228, 3273, 3318, | H | H | H | F | F |
| 3229, 3274, 3319, | H | F | H | H | F |
| 3230, 3275, 3320, | F | F | H | H | F |
| 3231, 3276, 3321, | H | H | F | F | F |
| 3232, 3277, 3322, | H | F | H | F | F |
| 3233, 3278, 3323, | H | F | F | F | F |
| 3234, 3279, 3324, | F | F | H | F | F |
| 3235, 3280, 3325, | F | F | F | F | F |
| 3236, 3281, 3326, | H | H | H | H | CF₃ |
| 3237, 3282, 3327, | H | H | H | F | CF₃ |
| 3238, 3283, 3328, | H | F | H | H | CF₃ |
| 3239, 3284, 3329, | F | F | H | H | CF₃ |
| 3240, 3285, 3330, | H | H | F | F | CF₃ |
| 3241, 3286, 3331, | H | F | H | F | CF₃ |
| 3242, 3287, 3332, | H | F | F | F | CF₃ |
| 3243, 3288, 3333, | F | F | H | F | CF₃ |
| 3244, 3289, 3334, | F | F | F | F | CF₃ |
| 3245, 3290, 3335, | H | H | H | H | OCF₃ |
| 3246, 3291, 3336, | H | H | H | F | OCF₃ |
| 3247, 3292, 3337, | H | F | H | H | OCF₃ |
| 3248, 3293, 3338, | F | F | H | H | OCF₃ |
| 3249, 3294, 3339, | H | H | F | F | OCF₃ |
| 3250, 3295, 3340, | H | F | H | F | OCF₃ |
| 3251, 3296, 3341, | H | F | F | F | OCF₃ |
| 3252, 3297, 3342, | F | F | H | F | OCF₃ |
| 3253, 3298, 3343, | F | F | F | F | OCF₃ |
| 3254, 3299, 3344, | H | H | H | H | Cl |
| 3255, 3300, 3345, | H | H | H | F | Cl |
| 3256, 3301, 3346, | H | F | H | H | Cl |
| 3257, 3302, 3347, | F | F | H | H | Cl |
| 3258, 3303, 3348, | H | H | F | F | Cl |
| 3259, 3304, 3349, | H | F | H | F | Cl |
| 3260, 3305, 3350, | H | F | F | F | Cl |
| 3261, 3306, 3351, | F | F | H | F | Cl |
| 3262, 3307, 3352, | F | F | F | F | Cl |
| 3263, 3308, 3353, | H | H | H | H | CN |
| 3264, 3309, 3354, | H | H | H | F | CN |
| 3265, 3310, 3355, | H | F | H | H | CN |
| 3266, 3311, 3356, | F | F | H | H | CN |
| 3267, 3312, 3357, | H | H | F | F | CN |
| 3268, 3313, 3358, | H | F | H | F | CN |
| 3269, 3314, 3359, | H | F | F | F | CN |
| 3270, 3315, 3360, | F | F | H | F | CN |
| 3271, 3316, 3361, | F | F | F | F | CN |

where alk(en)yl is selected from: CH₃, C₂H₅, C₃H₇,
C₄H₉, C₅H₁₁, CH═CH₂, CH═CH—CH₃ and CH═CH—C₃H₇.
Examples 3227 to 3271

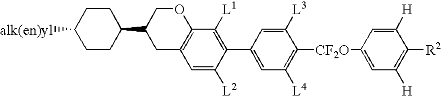

Examples 3272 to 3316

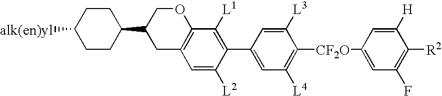

Examples 3317 to 3361

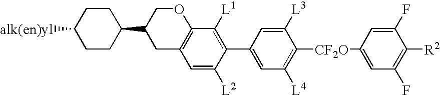

| Examples | L¹ | L² | L³ | L⁴ | R² |
|---|---|---|---|---|---|
| 3362, 3407, 3452, | H | H | H | H | F |
| 3363, 3408, 3453, | H | H | H | F | F |
| 3364, 3409, 3454, | H | F | H | H | F |
| 3365, 3410; 3455, | F | F | H | H | F |
| 3366, 3411, 3456, | H | H | F | F | F |
| 3367, 3412, 3457, | H | F | H | F | F |
| 3368, 3413, 3458, | H | F | F | F | F |
| 3369, 3414, 3459, | F | F | H | F | F |
| 3370, 3415, 3460, | F | F | F | F | F |
| 3371, 3416, 3461, | H | H | H | H | CF₃ |
| 3372, 3417, 3462, | H | H | H | F | CF₃ |
| 3373, 3418, 3463, | H | F | H | H | CF₃ |
| 3374, 3419, 3464, | F | F | H | H | CF₃ |
| 3375, 3420, 3465, | H | H | F | F | CF₃ |
| 3376, 3421, 3466, | H | F | H | F | CF₃ |
| 3377, 3422, 3467, | H | F | F | F | CF₃ |
| 3378, 3423, 3468, | F | F | H | F | CF₃ |
| 3379, 3424, 3469, | F | F | F | F | CF₃ |
| 3380, 3425, 3470, | H | H | H | H | OCF₃ |
| 3381, 3426, 3471, | H | H | H | F | OCF₃ |
| 3382, 3427, 3472, | H | F | H | H | OCF₃ |
| 3383, 3428, 3473, | F | F | H | H | OCF₃ |
| 3384, 3429, 3474, | H | H | F | F | OCF₃ |
| 3385, 3430, 3475, | H | F | H | F | OCF₃ |
| 3386, 3431, 3476, | H | F | F | F | OCF₃ |
| 3387, 3432, 3477, | F | F | H | F | OCF₃ |
| 3388, 3433, 3478, | F | F | F | F | OCF₃ |
| 3389, 3434, 3479, | H | H | H | H | Cl |
| 3390, 3435, 3480, | H | H | H | F | Cl |
| 3391, 3436, 3481, | H | F | H | H | Cl |
| 3392, 3437, 3482, | F | F | H | H | Cl |
| 3393, 3438, 3483, | H | H | F | F | Cl |
| 3394, 3439, 3484, | H | F | H | F | Cl |
| 3395, 3440, 3485, | H | F | F | F | Cl |
| 3396, 3441, 3486, | F | F | H | F | Cl |
| 3397, 3442, 3487, | F | F | F | F | Cl |
| 3398, 3443, 3488, | H | H | H | H | CN |
| 3399, 3444, 3489, | H | H | H | F | CN |
| 3400, 3445, 3490, | H | F | H | H | CN |
| 3401, 3446, 3491, | F | F | H | H | CN |
| 3402, 3447, 3492, | H | H | F | F | CN |
| 3403, 3448, 3493, | H | F | H | F | CN |
| 3404, 3449, 3494, | H | F | F | F | CN |
| 3405, 3450, 3495, | F | F | H | F | CN |
| 3406, 3451, 3496, | F | F | F | F | CN |

where alk(en)yl is selected from: CH₃, C₂H₅, C₃H₇,
C₄H₉, C₅H₁₁, CH═CH₂, CH═CH—CH₃ and CH═CH—C₃H₇.
Examples 3362 to 3406

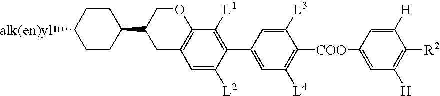

Examples 3407 to 3451

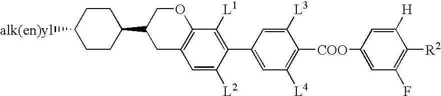

Examples 3452 to 3496

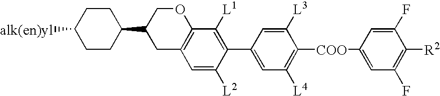

| Examples | L¹ | L² | L³ | L⁴ | R² |
|---|---|---|---|---|---|
| 3497, 3542, 3587, | H | H | H | H | F |
| 3498, 3543, 3588, | H | H | H | F | F |
| 3499, 3544, 3589, | H | F | H | H | F |
| 3500, 3545, 3590, | F | F | H | H | F |
| 3501, 3546, 3591, | H | H | F | F | F |
| 3502, 3547, 3592, | H | F | H | F | F |
| 3503, 3548, 3593, | H | F | F | F | F |
| 3504, 3549, 3594, | F | F | H | F | F |
| 3505, 3550, 3595, | F | F | F | F | F |
| 3506, 3551, 3596, | H | H | H | H | CF₃ |
| 3507, 3552, 3597, | H | H | H | F | CF₃ |
| 3508, 3553, 3598, | H | F | H | H | CF₃ |
| 3509, 3554, 3599, | F | F | H | H | CF₃ |
| 3510, 3555, 3600, | H | H | F | F | CF₃ |
| 3511, 3556, 3601, | H | F | H | F | CF₃ |
| 3512, 3557, 3602, | H | F | F | F | CF₃ |
| 3513, 3558, 3603, | F | F | H | F | CF₃ |
| 3514, 3559, 3604, | F | F | F | F | CF₃ |
| 3515, 3560, 3605, | H | H | H | H | OCF₃ |
| 3516, 3561, 3606, | H | H | H | F | OCF₃ |
| 3517, 3562, 3607, | H | F | H | H | OCF₃ |
| 3518, 3563, 3608, | F | F | H | H | OCF₃ |
| 3519, 3564, 3609, | H | H | F | F | OCF₃ |
| 3520, 3565, 3610, | H | F | H | F | OCF₃ |
| 3521, 3566, 3611, | H | F | F | F | OCF₃ |
| 3522, 3567, 3612, | F | F | H | F | OCF₃ |
| 3523, 3566, 3613, | F | F | F | F | OCF₃ |
| 3524, 3569, 3614, | H | H | H | H | Cl |
| 3525, 3570, 3615, | H | H | H | F | Cl |
| 3526, 3571, 3616, | H | F | H | H | Cl |
| 3527, 3572, 3617, | F | F | H | H | Cl |
| 3528, 3573, 3618, | H | H | F | F | Cl |
| 3529, 3574, 3619, | H | F | H | F | Cl |
| 3530, 3575, 3620, | H | F | F | F | Cl |
| 3531, 3576, 3621, | F | F | H | F | Cl |
| 3532, 3577, 3622, | F | F | F | F | Cl |
| 3533, 3578, 3623, | H | H | H | H | CN |
| 3534, 3579, 3624, | H | H | H | F | CN |
| 3535, 3580, 3625, | H | F | H | H | CN |
| 3536, 3581, 3626, | F | F | H | H | CN |
| 3537, 3582, 3627, | H | H | F | F | CN |
| 3538, 3583, 3628, | H | F | H | F | CN |
| 3539, 3584, 3629, | H | F | F | F | CN |
| 3540, 3585, 3630, | F | F | H | F | CN |
| 3541, 3586, 3631, | F | F | F | F | CN |

where alk(en)yl is selected from: CH₃, C₂H₅, C₃H₇,
C₄H₉, C₅H₁₁, CH═CH₂, CH═CH—CH₃ and CH═CH—C₃H₇.
Examples 3497 to 3541

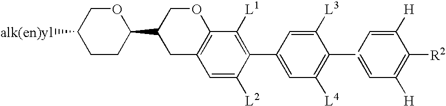

Examples 3542 to 3586

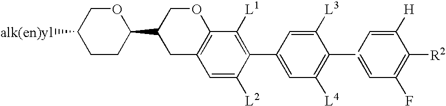

Examples 3587 to 3631

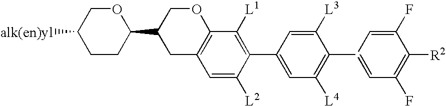

Examples 3632 to 3676

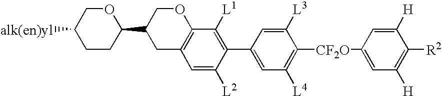

Examples 3677 to 3721

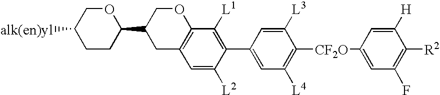

Examples 3722 to 3766

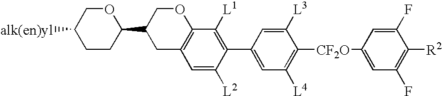

| Examples | | | L¹ | L² | L³ | L⁴ | R² |
|---|---|---|---|---|---|---|---|
| 3632, | 3677, | 3722, | H | H | H | H | F |
| 3633, | 3678, | 3723, | H | H | H | F | F |
| 3634, | 3679, | 3724, | H | F | H | H | F |
| 3635, | 3680, | 3725, | F | F | H | H | F |
| 3636, | 3681, | 3726, | H | H | F | F | F |
| 3637, | 3682, | 3727, | H | F | H | F | F |
| 3638, | 3683, | 3728, | H | F | F | F | F |
| 3639, | 3684, | 3729, | F | F | H | F | F |
| 3640, | 3685, | 3730, | F | F | F | F | F |
| 3641, | 3686, | 3731, | H | H | H | H | CF₃ |
| 3642, | 3687, | 3732, | H | H | H | F | CF₃ |
| 3643, | 3688, | 3733, | H | F | H | H | CF₃ |
| 3644, | 3689, | 3734, | F | F | H | H | CF₃ |
| 3645, | 3690, | 3735, | H | H | F | F | CF₃ |
| 3646, | 3691, | 3736, | H | F | H | F | CF₃ |
| 3647, | 3692, | 3737, | H | F | F | F | CF₃ |
| 3648, | 3693, | 3738, | F | F | H | F | CF₃ |
| 3649, | 3694, | 3739, | F | F | F | F | CF₃ |
| 3650, | 3695, | 3740, | H | H | H | H | OCF₃ |
| 3651, | 3696, | 3741, | H | H | H | F | OCF₃ |
| 3652, | 3697, | 3742, | H | F | H | H | OCF₃ |
| 3653, | 3698, | 3743, | F | F | H | H | OCF₃ |
| 3654, | 3699, | 3744, | H | H | F | F | OCF₃ |
| 3655, | 3700, | 3745, | H | F | H | F | OCF₃ |
| 3656, | 3701, | 3746, | H | F | F | F | OCF₃ |
| 3657, | 3702, | 3747, | F | F | H | F | OCF₃ |
| 3658, | 3703, | 3748, | F | F | F | F | OCF₃ |
| 3659, | 3704, | 3749, | H | H | H | H | Cl |
| 3660, | 3705, | 3750, | H | H | H | F | Cl |
| 3661, | 3706, | 3751, | H | F | H | H | Cl |
| 3662, | 3707, | 3752, | F | F | H | H | Cl |
| 3663, | 3708, | 3753, | H | H | F | F | Cl |
| 3664, | 3709, | 3754, | H | F | H | F | Cl |
| 3665, | 3710, | 3755, | H | F | F | F | Cl |
| 3666, | 3711, | 3756, | F | F | H | F | Cl |
| 3667, | 3712, | 3757, | F | F | F | F | Cl |
| 3668, | 3713, | 3758, | H | H | H | H | CN |
| 3669, | 3714, | 3759, | H | H | H | F | CN |
| 3670, | 3715, | 3760, | H | F | H | H | CN |
| 3671, | 3716, | 3761, | F | F | H | H | CN |
| 3672, | 3717, | 3762, | H | H | F | F | CN |
| 3673, | 3718, | 3763, | H | F | H | F | CN |
| 3674, | 3719, | 3764, | H | F | F | F | CN |
| 3675, | 3720, | 3765, | F | F | H | F | CN |
| 3676, | 3721, | 3766, | F | F | F | F | CN |

where alk(en)yl is selected from: CH₃, C₂H₅, C₃H₇, C₄H₉, C₅H₁₁, CH═CH₂, CH═CH—CH₃ and CH═CH—C₃H₇.

Examples 3767 to 3811

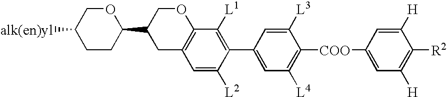

Examples 3812 to 3856

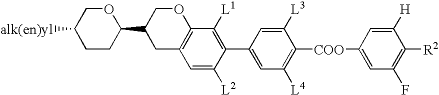

Examples 3857 to 3901

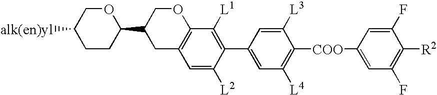

| Examples | | | L¹ | L² | L³ | L⁴ | R² |
|---|---|---|---|---|---|---|---|
| 3767, | 3812, | 3857, | H | H | H | H | F |
| 3768, | 3813, | 3858, | H | H | H | F | F |
| 3769, | 3814, | 3859, | H | F | H | H | F |
| 3770, | 3815, | 3860, | F | F | H | H | F |
| 3771, | 3816, | 3861, | H | H | F | F | F |
| 3772, | 3817, | 3862, | H | F | H | F | F |
| 3773, | 3818, | 3863, | H | F | F | F | F |
| 3774, | 3819, | 3854, | F | F | H | F | F |
| 3775, | 3820, | 3865, | F | F | F | F | F |
| 3776, | 3821, | 3866, | H | H | H | H | CF₃ |
| 3777, | 3822, | 3867, | H | H | H | F | CF₃ |
| 3778, | 3823, | 3868, | H | F | H | H | CF₃ |
| 3779, | 3824, | 3859, | F | F | H | H | CF₃ |
| 3780, | 3825, | 3870, | H | H | F | F | CF₃ |
| 3781, | 3826, | 3871, | H | F | H | F | CF₃ |
| 3782, | 3827, | 3872, | H | F | F | F | CF₃ |
| 3783, | 3828, | 3873, | F | F | H | F | CF₃ |
| 3784, | 3829, | 3874, | F | F | F | F | CF₃ |
| 3785, | 3830, | 3875, | H | H | H | H | OCF₃ |
| 3786, | 3831, | 3876, | H | H | H | F | OCF₃ |
| 3787, | 3832, | 3877, | H | F | H | H | OCF₃ |
| 3788, | 3833, | 3878, | F | F | H | H | OCF₃ |
| 3789, | 3834, | 3879, | H | H | F | F | OCF₃ |
| 3790, | 3835, | 3880, | H | F | H | F | OCF₃ |
| 3791, | 3836, | 3881, | H | F | F | F | OCF₃ |
| 3792, | 3837, | 3882, | F | F | H | F | OCF₃ |
| 3793, | 3838, | 3883, | F | F | F | F | OCF₃ |
| 3794, | 3839, | 3884, | H | H | H | H | Cl |
| 3795, | 3840, | 3885, | H | H | H | F | Cl |
| 3796, | 3841, | 3886, | H | F | H | H | Cl |
| 3797, | 3842, | 3887, | F | F | H | H | Cl |
| 3798, | 3843, | 3888, | H | H | F | F | Cl |
| 3799, | 3844, | 3889, | H | F | H | F | Cl |
| 3800, | 3845, | 3890, | H | F | F | F | Cl |
| 3801, | 3846, | 3891, | F | F | H | F | Cl |
| 3802, | 3847, | 3892, | F | F | F | F | Cl |
| 3803, | 3848, | 3893, | H | H | H | H | CN |
| 3804, | 3849, | 3894, | H | H | H | F | CN |
| 3805, | 3850, | 3895, | H | F | H | H | CN |
| 3806, | 3851, | 3896, | F | F | H | H | CN |
| 3807, | 3852, | 3897, | H | H | F | F | CN |
| 3808, | 3853, | 3898, | H | F | H | F | CN |
| 3809, | 3854, | 3899, | H | F | F | F | CN |
| 3810, | 3855, | 3900, | F | F | H | F | CN |
| 3811, | 3856, | 3901, | F | F | F | F | CN |

where alk(en)yl is selected from: CH₃, C₂H₅, C₃H₇, C₄H₉, C₅H₁₁, CH═CH₂, CH═CH—CH₃ and CH═CH—C₃H₇.

Examples 3902 to 3946

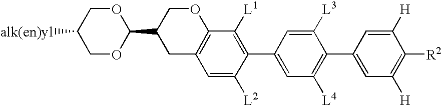

Examples 3947 to 3991

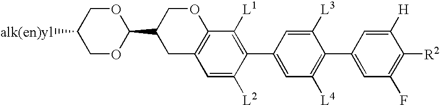

Examples 3992 to 4036

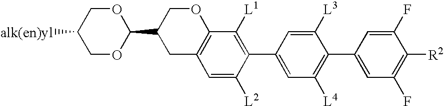

| Examples | | | L¹ | L² | L³ | L⁴ | R² |
|---|---|---|---|---|---|---|---|
| 3902, | 3947, | 3992, | H | H | H | H | F |
| 3903, | 3948, | 3993, | H | H | H | F | F |
| 3904, | 3949, | 3994, | H | F | H | H | F |
| 3905, | 3950, | 3995, | F | F | H | H | F |
| 3906, | 3951, | 3996, | H | H | F | F | F |
| 3907, | 3952, | 3997, | H | F | H | F | F |
| 3908, | 3953, | 3998, | H | F | F | F | F |
| 3909, | 3954, | 3999, | F | F | H | F | F |
| 3910, | 3955, | 4000, | F | F | F | F | F |
| 3911, | 3956, | 4001, | H | H | H | H | CF₃ |
| 3912, | 3957, | 4002, | H | H | H | F | CF₃ |
| 3913, | 3958, | 4003, | H | F | H | H | CF₃ |
| 3914, | 3959, | 4004, | F | F | H | H | CF₃ |
| 3915, | 3960, | 4005, | H | H | F | F | CF₃ |
| 3916, | 3961, | 4006, | H | F | H | F | CF₃ |
| 3917, | 3962, | 4007, | H | F | F | F | CF₃ |
| 3918, | 3963, | 4008, | F | F | H | F | CF₃ |
| 3019, | 3964, | 4009, | F | F | F | F | CF₃ |
| 3920, | 3965, | 4010, | H | H | H | H | OCF₃ |
| 3921, | 3966, | 4011, | H | H | H | F | OCF₃ |
| 3922, | 3967, | 4012, | H | F | H | H | OCF₃ |
| 3923, | 3968, | 4013, | F | F | H | H | OCF₃ |
| 3924, | 3969, | 4014, | H | H | F | F | OCF₃ |
| 3925, | 3970, | 4015, | H | F | H | F | OCF₃ |
| 3926, | 3971, | 4016, | H | F | F | F | OCF₃ |
| 3927, | 3972, | 4017, | F | F | H | F | OCF₃ |
| 3928, | 3973, | 4018, | F | F | F | F | OCF₃ |
| 3929, | 3974, | 4019, | H | H | H | H | Cl |
| 3930, | 3975, | 4020, | H | H | H | F | Cl |
| 3931, | 3976, | 4021, | H | F | H | H | Cl |
| 3932, | 3977, | 4022, | F | F | H | H | Cl |
| 3933, | 3978, | 4023, | H | H | F | F | Cl |
| 3934, | 3979, | 4024, | H | F | H | F | Cl |
| 3935, | 3980, | 4025, | H | F | F | F | Cl |
| 3936, | 3981, | 4026, | F | F | H | F | Cl |
| 3937, | 3982, | 4027, | F | F | F | F | Cl |
| 3938, | 3983, | 4028, | H | H | H | H | CN |
| 3939, | 3984, | 4029, | H | H | H | F | CN |
| 3940, | 3985, | 4030, | H | F | H | H | CN |
| 3941, | 3986, | 4031, | F | F | H | H | CN |
| 3942, | 3987, | 4032, | H | H | F | F | CN |
| 3943, | 3988, | 4033, | H | F | H | F | CN |
| 3944, | 3989, | 4034, | H | F | F | F | CN |
| 3945, | 3990, | 4035, | F | F | H | F | CN |
| 3946, | 3991, | 4036, | F | F | F | F | CN |

where alk(en)yl is selected from: CH₃, C₂H₅, C₃H₇, C₄H₉, C₅H₁₁, CH═CH₂, CH═CH—CH₃ and CH═CH—C₃H₇.

Examples 4037 to 4081

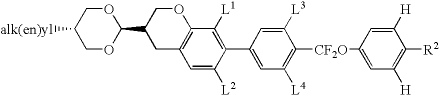

Examples 4082 to 4126

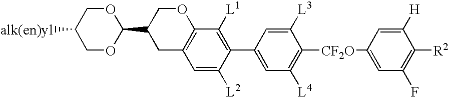

Examples 4127 to 4171

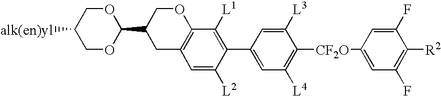

| Examples | | | L¹ | L² | L³ | L⁴ | R² |
|---|---|---|---|---|---|---|---|
| 4037, | 4082, | 4127, | H | H | H | H | F |
| 4038, | 4083, | 4128, | H | H | H | F | F |
| 4039, | 4084, | 4129, | H | F | H | H | F |
| 4040, | 4085, | 4130, | F | F | H | H | F |
| 4041, | 4086, | 4131, | H | H | F | F | F |
| 4042, | 4087, | 4132, | H | F | H | F | F |
| 4043, | 4088, | 4133, | H | F | F | F | F |
| 4044, | 4089, | 4134, | F | F | H | F | F |
| 4045, | 4090, | 4135, | F | F | F | F | F |
| 4046, | 4091, | 4136, | H | H | H | H | CF₃ |
| 4047, | 4092, | 4137, | H | H | H | F | CF₃ |
| 4048, | 4093, | 4138, | H | F | H | H | CF₃ |
| 4049, | 4094, | 4139, | F | F | H | H | CF₃ |
| 4050, | 4095, | 4140, | H | H | F | F | CF₃ |
| 4051, | 4096, | 4141, | H | F | H | F | CF₃ |
| 4052, | 4097, | 4142, | H | F | F | F | CF₃ |
| 4053, | 4098, | 4143, | F | F | H | F | CF₃ |
| 4054, | 4099, | 4144, | F | F | F | F | CF₃ |
| 4055, | 4100, | 4145, | H | H | H | H | OCF₃ |
| 4056, | 4101, | 4146, | H | H | H | F | OCF₃ |
| 4057, | 4102, | 4147, | H | F | H | H | OCF₃ |
| 4058, | 4103, | 4148, | F | F | H | H | OCF₃ |
| 4059, | 4104, | 4149, | H | H | F | F | OCF₃ |
| 4060, | 4105, | 4150, | H | F | H | F | OCF₃ |
| 4061, | 4106, | 4151, | H | F | F | F | OCF₃ |
| 4062, | 4107, | 4152, | F | F | H | F | OCF₃ |
| 4063, | 4108, | 4153, | F | F | F | F | OCF₃ |
| 4064, | 4109, | 4154, | H | H | H | H | Cl |
| 4065, | 4110, | 4155, | H | H | H | F | Cl |
| 4066, | 4111, | 4156, | H | F | H | H | Cl |
| 4067, | 4112, | 4157, | F | F | H | H | Cl |
| 4068, | 4113, | 4158, | H | H | F | F | Cl |
| 4069, | 4114, | 4159, | H | F | H | F | Cl |
| 4070, | 4115, | 4160, | H | F | F | F | Cl |
| 4071, | 4116, | 4161, | F | F | H | F | Cl |
| 4072, | 4117, | 4162, | F | F | F | F | Cl |
| 4073, | 4118, | 4163, | H | H | H | H | CN |
| 4074, | 4119, | 4164, | H | H | H | F | CN |
| 4075, | 4120, | 4165, | H | F | H | H | CN |
| 4076, | 4121, | 4166, | F | F | H | H | CN |
| 4077, | 4122, | 4167, | H | H | F | F | CN |
| 4078, | 4123, | 4168, | H | F | H | F | CN |
| 4079, | 4124, | 4169, | H | F | F | F | CN |
| 4080, | 4125, | 4170, | F | F | H | F | CN |
| 4081, | 4126, | 4171, | F | F | F | F | CN |

where alk(en)yl is selected from: CH₃, C₂H₅, C₃H₇, C₄H₉, C₅H₁₁, CH═CH₂, CH═CH—CH₃ and CH═CH—C₃H₇.

Examples 4172 to 4216

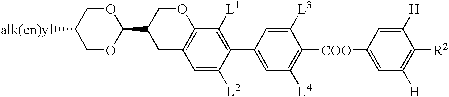

Examples 4217 to 4261

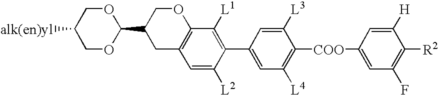

Examples 4262 to 4306

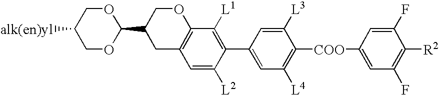

| Examples | | | L¹ | L² | L³ | L⁴ | R² |
|---|---|---|---|---|---|---|---|
| 4172, | 4217, | 4262, | H | H | H | H | F |
| 4173, | 4218, | 4263, | H | H | H | F | F |
| 4174, | 4219, | 4264, | H | F | H | H | F |
| 4175, | 4220, | 4265, | F | F | H | H | F |
| 4176, | 4221, | 4266, | H | H | F | F | F |
| 4177, | 4222, | 4267, | H | F | H | F | F |
| 4178, | 4223, | 4268, | H | F | F | F | F |
| 4179, | 4224, | 4269, | F | F | H | F | F |
| 4180, | 4225, | 4270, | F | F | F | F | F |
| 4181, | 4226, | 4271, | H | H | H | H | CF₃ |
| 4182, | 4227, | 4272, | H | H | H | F | CF₃ |
| 4183, | 4228, | 4273, | H | F | H | H | CF₃ |
| 4184, | 4229, | 4274, | F | F | H | H | CF₃ |
| 4185, | 4230, | 4275, | H | H | F | F | CF₃ |
| 4186, | 4231, | 4276, | H | F | H | F | CF₃ |
| 4187, | 4232, | 4277, | H | F | F | F | CF₃ |
| 4188, | 4233, | 4278, | F | F | H | F | CF₃ |
| 4189, | 4234, | 4279, | F | F | F | F | CF₃ |
| 4190, | 4235, | 4280, | H | H | H | H | OCF₃ |
| 4191, | 4236, | 4281, | H | H | H | F | OCF₃ |
| 4192, | 4237, | 4282, | H | F | H | H | OCF₃ |
| 4193, | 4238, | 4283, | F | F | H | H | OCF₃ |
| 4194, | 4239, | 4284, | H | H | F | F | OCF₃ |
| 4195 | 4240, | 4285, | H | F | H | F | OCF₃ |
| 4196, | 4241, | 4286, | H | F | F | F | OCF₃ |
| 4197, | 4242, | 4287, | F | F | H | F | OCF₃ |
| 4198, | 4243, | 4288, | F | F | F | F | OCF₃ |
| 4199, | 4244, | 4289, | H | H | H | H | Cl |
| 4200, | 4245, | 4290, | H | H | H | F | Cl |
| 4201, | 4246, | 4291, | H | F | H | H | Cl |
| 4202, | 4247, | 4292, | F | F | H | H | Cl |
| 4203, | 4248, | 4293, | H | H | F | F | Cl |
| 4204, | 4249, | 4294, | H | F | H | F | Cl |
| 4205, | 4250, | 4295, | H | F | F | F | Cl |
| 4206, | 4251, | 4296, | F | F | H | F | Cl |
| 4207, | 4252, | 4297, | F | F | F | F | Cl |
| 4208, | 4253, | 4298, | H | H | H | H | CN |
| 4209, | 4254, | 4299, | H | H | H | F | CN |
| 4210, | 4255, | 4300, | H | F | H | H | CN |
| 4211, | 4256, | 4301, | F | F | H | H | CN |
| 4212, | 4257, | 4302, | H | H | F | F | CN |
| 4213, | 4258, | 4303, | H | F | H | F | CN |
| 4214, | 4259, | 4304, | H | F | F | F | CN |
| 4215, | 4260, | 4305, | F | F | H | F | CN |
| 4216, | 4261, | 4306, | F | F | F | F | CN |

where alk(en)yl is selected from: CH₃, C₂H₅, C₃H₇, C₄H₉, C₅H₁₁, CH═CH₂, CH═CH—CH₃ and CH═CH—C₃H₇.

### EXAMPLE 4307

### 7,8-Difluoro-6-(4-pentylcyclohexyl)-2-propylchromane

### 1st Step: 7,8-Difluoro-6-(4-pentylcyclohexyl)-2-propyl-2H-chromene

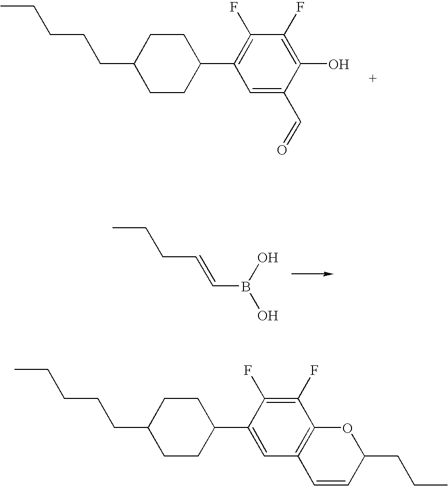

As described by Q. Wang, N. G. Finn, Org. Lett. 2000, pp. 4063-4065, 5 g (16.1 mmol) of 3,4-difluoro-2-hydroxy-5-(4-pentylcyclohexyl)benzaldehyde and 3.8 g (33.3 mmol) of E-pent-1-enylboronic acid are left to stir for 48 hours at 90° C. in the presence of 0.6 ml of dibenzylamine in 80 ml of dioxane. After addition of water, the mixture is extracted with MTB ether and the combined organic phases are evaporated. The residue is chromatographed on silica gel using heptane/chlorobutane (10:1), giving 5.8 g (86%) of 7,8-difluoro-6-(4-pentylcyclohexyl)-2-propyl-2H-chromene as colourless oil.

### 2nd Step: 7,8-Difluoro-6-(4-pentylcyclohexyl)-2-propylchromane

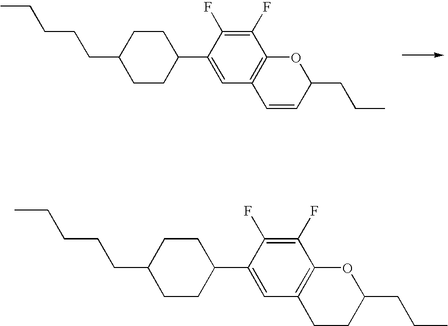

4.8 g (13.1 mmol) of 7,8-difluoro-6-(4-pentylcyclohexyl)-2-propyl-2H-chromene are dissolved in 50 ml of THF and hydrogenated to completion in the presence of palladium/activated carbon catalyst. The solution is filtered, the solvent is removed under reduced pressure, and the residue is recrystallised from ethanol, giving 3.4 g (71%) of 2-ethyl-6-(4-ethylcyclohexyl)-7,8-difluorochromane as colourless crystals.

T_{g}−53 C 55 N (15.3) I

Δ∈=−7.3

Δn=0.0812

The following compounds of Examples 43038 to 4333 are obtained analogously to Example 4307 using the corresponding precursors.

### EXAMPLE 4308

### 7,8-Difluoro-2-pentyl-6-(4-pentylcyclohexyl)chromane

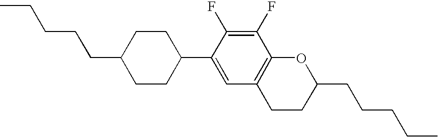

C 34 N (25.2) I

Δ∈=−6.8

Δn=0.0746

### EXAMPLE 4309

### 7,8-Difluoro-2-(4-pentylcyclohexyl)-6-(4′-propylbicyclohexyl-4-yl)chromane

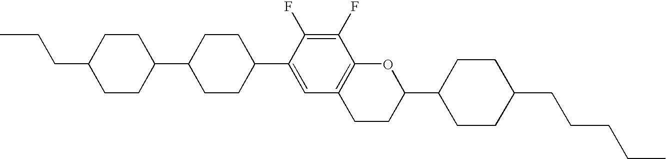

C 143 SmA (139) N 277.5 I

Δ∈=−−5.4

Δn=0.0712

### EXAMPLE 4310

### 7,8-Difluoro-6-(4-pentylphenyl)-2-propylchromane

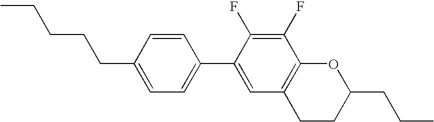

C 51 I

Δ∈=−7.0

Δn=0.1407

### EXAMPLE 4311

### 7,8-Difluoro-2-methyl-6-(4-butylcycohexyl)chromane

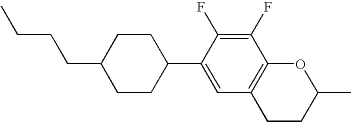

C 75 I

Δ∈=−6.8

Δn=0.0797

### EXAMPLE 4312

### 7,8-Difluoro-2-methyl-6-(4′-propylbicyclohexyl-4-yl)chromane

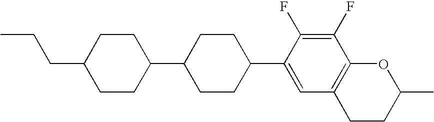

C 150 N 157 I

Δ∈=−6.7

Δn=0.0758

### EXAMPLE 4313

### 2-(4-Butylcyclohexyl)-6-ethoxy-7,8-difluorochromane

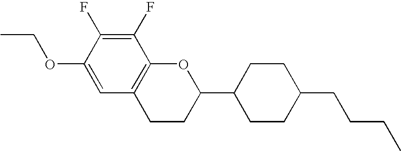

C 100 I

Δ∈=−10.8

Δn=0.0834

### EXAMPLE 4314

### 6-Ethoxy-7,8-difluoro-2-(4′-propylbicyclohexyl-4-yl)chromane

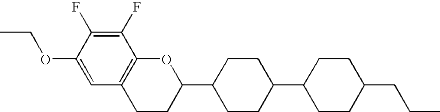

C 155 N 187 I

Δn=0.0766

### EXAMPLE 4315

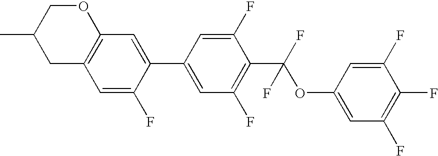

C 119 I

### EXAMPLE 4316

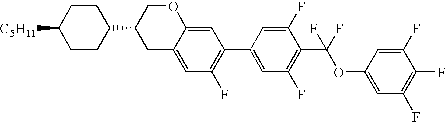

C 81 SmA (75) N 177

Δ∈=31.3

Δn=0.1430

### EXAMPLE 4317

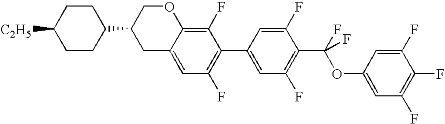

C 110 N 1457 I

Δ∈=36.9

Δn=0.1358

### EXAMPLE 4318

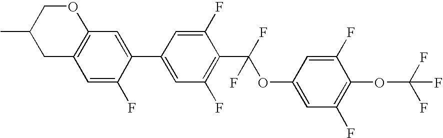

C 102 I

Δ∈=42.3

Δn=0.1354

### EXAMPLE 4319

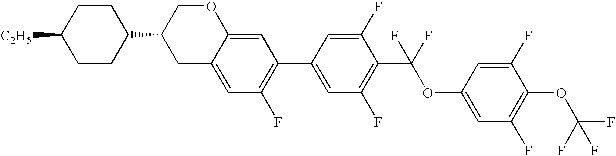

C 99 SmA 126 N 174 I

Δ∈=41.6

Δn=0.1366

### EXAMPLE 4320

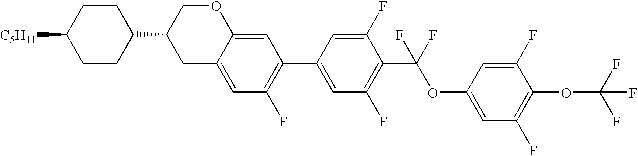

C 89 SmA 150 N 186.6 I

Δ∈=34.3

Δn=0.1351

### EXAMPLE 4321

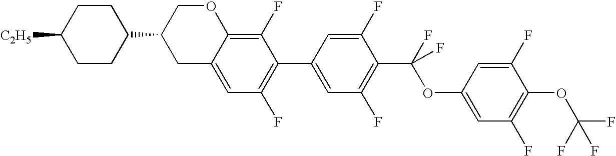

C 98 SmA 110 N 157.8 I

Δ∈=37.9

Δn=0.1295

### EXAMPLE 4332

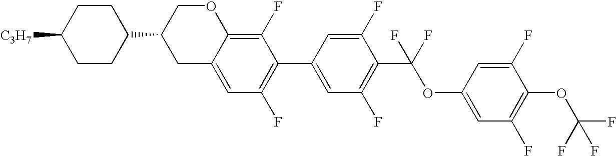

C 88 SmA 124 N 176.8 I

Δ∈=39.1

Δn=0.1317

### EXAMPLE 4323

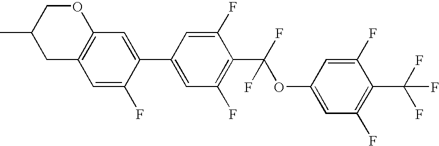

C 121 I

### EXAMPLE 4324

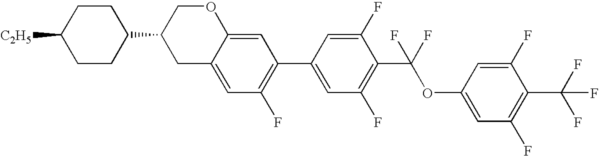

C 122 N 152.3 I

Δ∈=46.0

Δn=0.1406

### EXAMPLE 4325

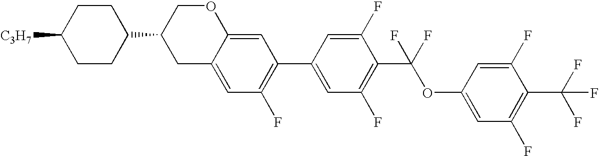

C 107 SmA (83) N 169.4 I

Δ∈=44.2

Δn=0.1404

### EXAMPLE 4326

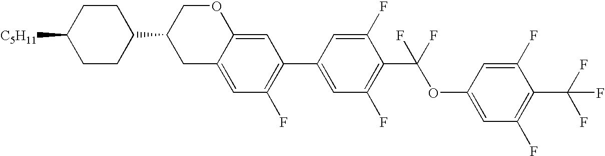

C 83 SmA 104 N 167.7 I

Δ∈=42.6

Δn=0.1392

### EXAMPLE 4327

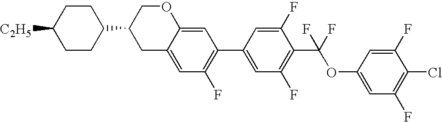

C 134 N 180.7 I

Δ∈=32.3

Δn=0.1498

### EXAMPLE 4328

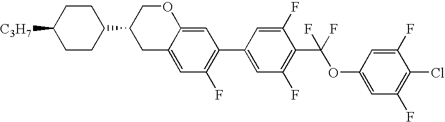

C 110 N 197.8 I

Δ∈=32.5

Δn=0.1535

### EXAMPLE 4329

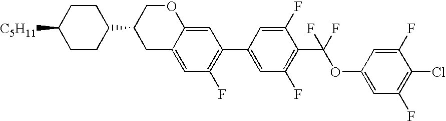

C 106 SmA (104) N 193.8 I

Δ∈=30.3

Δn=0.1501

### EXAMPLE 4330

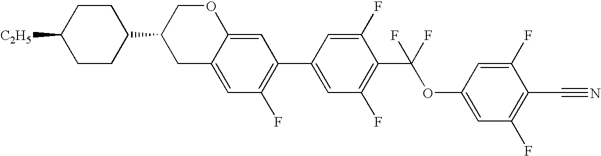

C 145 N 204.1 I

Δn=0.1623

### EXAMPLE 4331

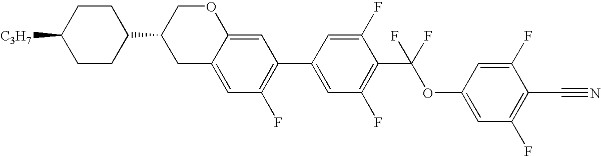

C 142 N 215.2 I

Δn=0.1559

### EXAMPLE 4332

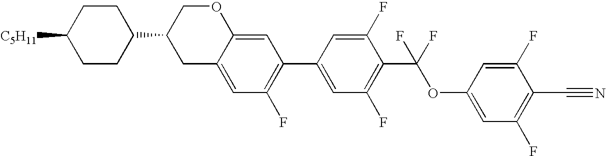

C 132 N 208.9 I

### EXAMPLE 4333

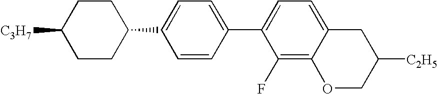

C 104 N 190.7 I

### EXAMPLE 4334

A liquid-crystal mixture comprising

| | |
|---|---|
| BCH—3F•F | 10.80% |
| BCH—5F•F | 9.00% |
| ECCP—3OCF3 | 4.50% |
| ECCP—5OCF3 | 4.50% |
| CBC—33F | 1.80% |
| CBC—53F | 1.80% |
| CBC—55F | 1.80% |
| PCH—6F | 7.20% |
| PCH—7F | 5.40% |
| CCP—2OCF3 | 7.20% |
| CCP—3OCF3 | 10.80% |
| CCP—4OCF3 | 6.30% |
| CCP—5OCF3 | 9.90% |
| PCH—5F | 9.00% |
| Compound of Example 26 | 10.00% |

has the following properties;

| clearing point: | +92.3° C. |
|---|---|
| Δε: | +7.3 |
| Δn: | +0.1012 |

### EXAMPLE 4335

A liquid-crystal mixture comprising

| | |
|---|---|
| BCH—3F•F | 10.80% |
| BCH—5F•F | 9.00% |
| ECCP—3OCF3 | 4.50% |
| ECCP—5OCF3 | 4.50% |
| CBC—33F | 1.80% |
| CBC—53F | 1.80% |
| CBC—55F | 1.80% |
| PCH—6F | 7.20% |
| PCH—7F | 5.40% |
| CCP—2OCF3 | 7.20% |
| CCP—3OCF3 | 10.80% |
| CCP—4OCF3 | 6.30% |
| CCP—5OCF3 | 9.90% |
| PCH—5F | 9.00% |
| Compound of Example 28 | 10.00% |

has time following properties:

| clearing point: | +97.1° C. |
|---|---|
| Δε: | +8.4 |
| Δn: | +0.1028 |

### EXAMPLE 4336

A liquid-crystal mixture comprising

| | |
|---|---|
| BCH—3F•F | 10.80% |
| BCH—5F•F | 9.00% |
| ECCP—3OCF3 | 4.50% |
| ECCP—5OCF3 | 4.50% |
| CBC—33F | 1.80% |
| CBC—53F | 1.80% |
| CBC—55F | 1.80% |
| PCH—6F | 7.20% |
| PCH—7F | 5.40% |
| CCP—2OCF3 | 7.20% |
| CCP—3OCF3 | 10.80% |
| CCP—4OCF3 | 6.30% |
| CCP—5OCF3 | 9.90% |
| PCH—5F | 9.00% |
| Compound of Example 18 | 10.00% |

has the following properties:

| clearing point: | +80.7° C. |
|---|---|
| Δε: | +5.8 |
| Δn: | +0.0916 |

### EXAMPLE 4337

A liquid-crystal mixture comprising

| | |
|---|---|
| PCH—3O1 | 9.00% |
| PCH—3O2 | 9.00% |
| CCH—3O1 | 29.70% |
| CCN—47 | 9.90% |
| CCN—55 | 9.00% |
| CBC—33F | 4.50% |
| CBC—53F | 4.50% |
| CBC—55F | 4.50% |
| CBC—33 | 4.50% |
| CBC—53 | 5.40% |
| Compound of Example 18 | 10.00% |

has the following properties:

| clearing point: | +72.0° C. |
|---|---|
| Δε: | −0.4 |

1. Chromane derivatives of the general formula (I) 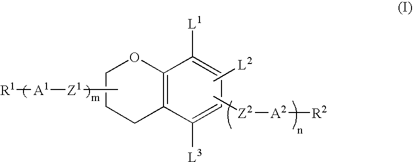
in which
R^{l} denotes H, halogen (F, Cl, Br, I), or a linear or branched, optionally chiral alkyl radical having 1 to 15 C atoms or alkenyl radical having 2 to 15 C atoms which is unsubstituted) monosubstituted by CN or CF₃ or at least monosubstituted by halogen, in which, in addition) one or more CH₂ groups may each) independently of one another) be replaced by —O—, —S—, —CO—, —CO—O—, —O—CO—, —O—CO—O—, —CH═CH—, —CH═CF—, —CF═CH—, —CF═CF—, —C≡C— or 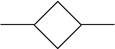
in such a way that hetero atoms are not linked directly to one another,
R² denotes, HF, Cl NCS, CN, SF₅, an alkyl or alkoxy radical having 1 to 15 C atoms, an alkenyl or alkenyloxy radical heaving 2 to 15 C atoms, an alkyl or alkoxy radical having 1 to 15 C atoms which is substituted by one or more fluorine atoms, or an alkenyl or alkenyloxy radical having 2 to 15 C atoms which is substituted by one or more fluorine atoms,
A¹, A², each, independently of one another, identically or differently, denote
a) trans-1,4-cyclohexylene, in which, in addition, one or more non-adjacent CH₂ groups may be replaced by —O—and/or —S—,
b) 1,4-phenylene, in which one or two CH groups may be replaced by N and in which, in addition, one or more H atoms may be replaced by F,
c) a radical from the group 1,4-bicyclo(2,2,2)octylene, piperidine-1,4-diyl, naphthalene-2,6-diyl, decalhydronaphthalene-2,6-diyl and 1,2,3,4-tetrahydronaphthalene-2,6-diyl, or
d) 1,4-cyclohexenylene,
Z¹, Z² each, independently of one another, identically or differently, denote —O—, —CH₂O—, —OCH₂—, —CO—O—, —O—CO—, —CF₂O—, —OCF₂—, —CF₂CF₂—, —CH₂CF₂—, —CF₂CH₂—, —CH₂CF₂O—, —OCF₂CH₂—, —CH₂CH₂—, —CH═CH—, CH═CF—, —CF═CH—, —CF═CF—, —CF═CF—CO—O—, —O—CO—CF═CF—, —C≡C— or a single bond,
L¹, L², L³ each, independently of one another, identically or differently, denote H, F, Cl, NCS, CN, SF₅, an alkyl or alkoxy radical having 1 to 15 C atoms which is substituted by one or more flourine atoms, or an alkenyl or alkenyloxy radical having 2 to 15 C atoms which is substituted by one or more fluorine atoms, preferably H, F, Cl or CN, and particularly preferably H or F,
m denotes 0, 1, 2, 3 or 4, preferably 0, 1, 2 or 3 and particularly preferably 0, 1 or 2, and
n denotes 1, 2, 3 or 4, preferably 1, 2 or 3 and particularly preferably 1 or 2,
but with the proviso that the sum (m+n)=1, 2, 3 or 4, preferably 1, 2 or 3 and particularly preferably 1 or 2,
and of the general formula (II) 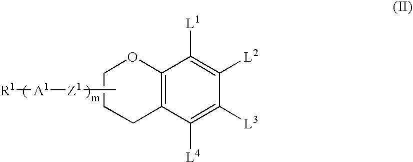
in which R¹, A¹ and Z¹ have the meanings indicated in relation to the formula (I),
L¹, L², L³ and L⁴ each, independently of one another, identically or differently, denote H, F, Cl, NCS, CN, SF₅ an allyl or alkoxy radical, having 1 to 15 C atoms which is substituted by one or more fluorine atoms, or an alkenyl or alkenyloxy radical having 2 to 15 C atoms which is substituted by one or more fluorine atoms, preferably H, F, Cl or CN, and particularly preferably H or F, where
one of the two radicals L² and L³ may additionally also adopt the meaning of R² in relation to the formula (1) and
L² and L³ together may also denote 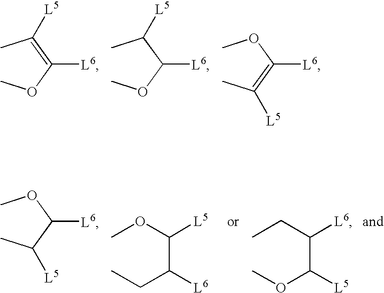
L⁵ and L⁶ each independently of one another, identically or differently, denote H, F, Cl or CN, and one of the two radicals additionally also denotes -(Z²-A²-)ₙR²,
but with the proviso that, if L⁵ and L⁶ each, independently of one another, identically or differently, denote H, F, Cl or CN, m=, 1, 2, 3 or 4, preferably 1, 2 or 3 and particularly preferably 1 or 2, and that, if one of the two radicals denotes -(Z²-A²)ₙR², m and n each, independently of one another, identically or
differently, are 0, 1, 2, 3 or 4, where the sum (m+n) 1, 2, 3 or 4, preferably 1, 2 or 3),
and chromene derivatives of the general formulae (III) and (IV) 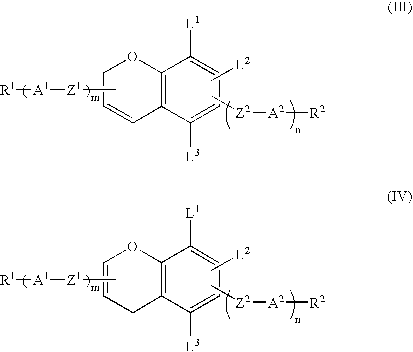
in which R¹, R², A¹, A², Z¹, Z², L¹, L², L³) m and n have the meanings indicated in relation to the formula (I),
and chromone derivatives of the general formulae (V) and (VI) 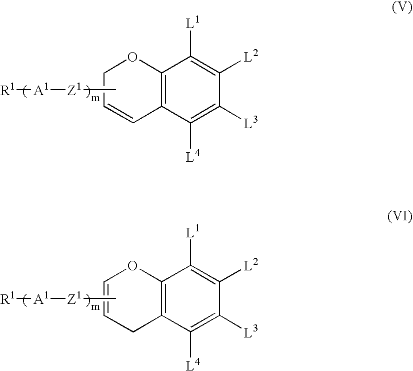
in which R¹, A¹, Z¹, L¹, L², L³, L⁴ and m have the meanings indicated in relation the formula (II).

2. Chromane derivatives according to claim 1, characterised in that the compounds of the sub-formula (I) are compounds of the following sub-formulae: 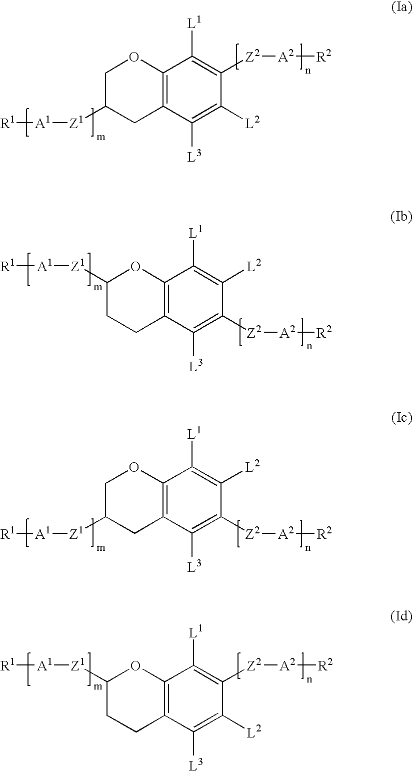
in which R¹, R², A¹, A², Z¹, Z², L¹, L², L³, m and n have the meanings indicated in relation to the formula (I) in claim 1.

3. Chromane derivatives according to claim 2, characterised in that the compounds of the sub-formula (Ia) are compounds of the following sub-formulae: 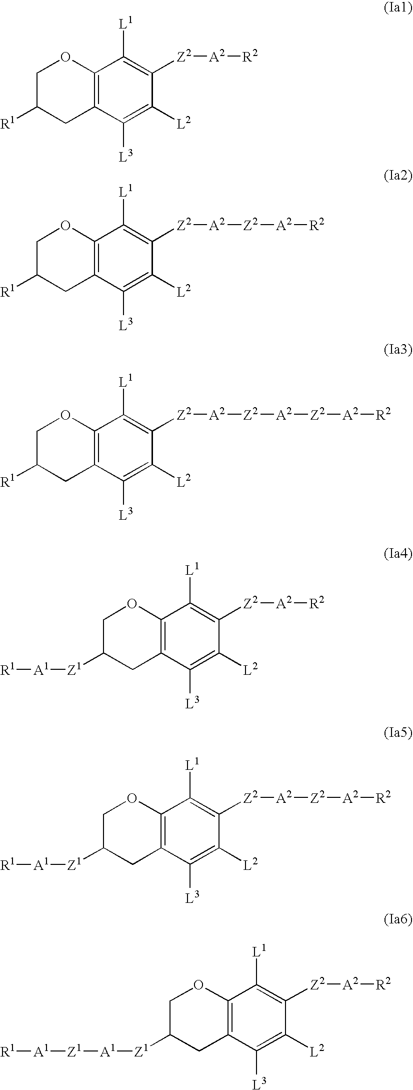
in which R¹, R², A¹, A², Z¹, Z², L¹, L² and L³ have the meanings indicated in relation to the formula (I).

4. Chromane derivatives according to claim 2, characterised in that the compounds of the sub-formula (Ib) are compounds of the following sub-formulae: 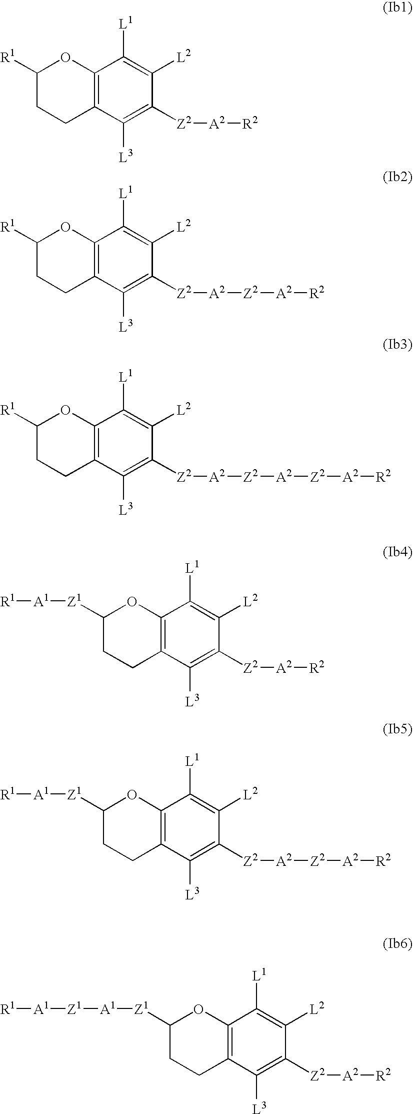
in R¹, R², A¹, A², Z¹, Z², L¹, L² and L³ have the meanings indicated in relation to the formula (I).

5. Chromane derivatives according to claim 1, characterised in that the compounds of the sub-formula (II) are compounds of the following sub-formulae. 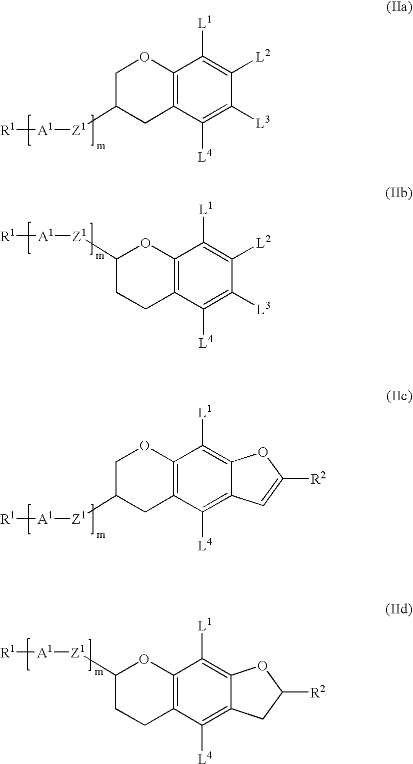
in which R¹, A¹, Z¹, Z², L¹, L², L³, L⁴ and m have the meanings indicated in relation to the formula (II) and R² has the meaning indicated in relation to the formula (I) in claim 1.

6. Chromane deriviatives according to claim 5, characterised in that the compounds of the sub-formula (IIa) are compounds of the following sub-formulae: 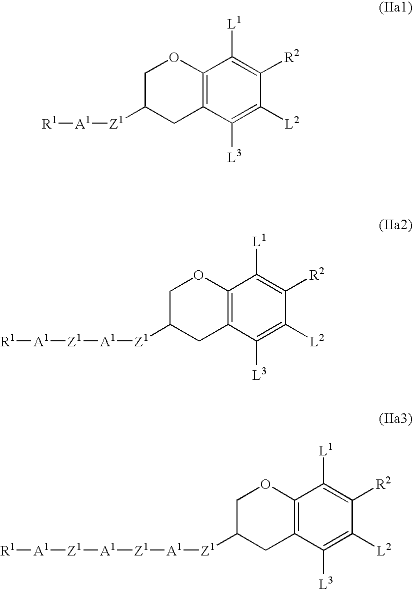
in which R¹, A¹, Z¹, L¹, L² and L³ have the meanings indicated in relation to the formula (II) and R² has the meanings indicated in relation to the formula (I).

7. Chromane derivatives of the general formulae (IIa1) to (IIa3) according to claim 6, characterised in that they have the following structures: 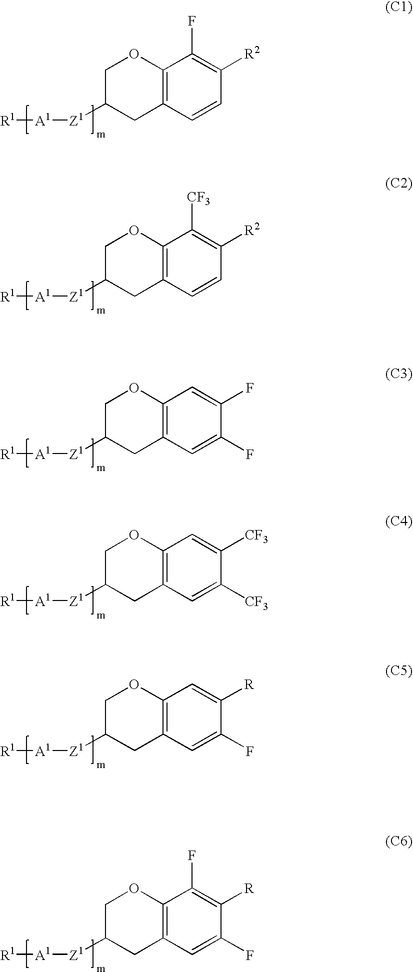
in which R¹, A¹ and Z¹ adopt the meanings indicated in relation to the formula (II), R² adopts the meanings indicated in relation to the formula (I), and m=1, 2 or 3.

8. Chromane derivatives according to claim 5, characterised in that the compounds of the sub-formula (IIb) are compounds of the following sub-formulae: 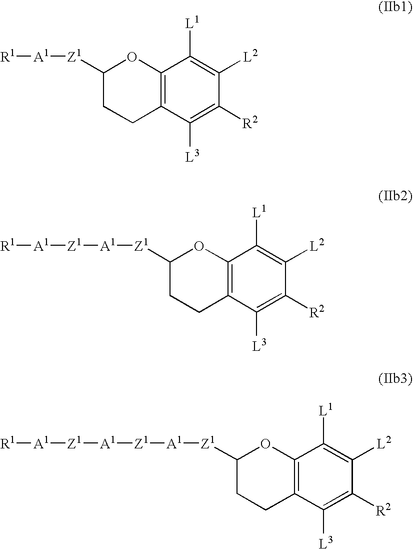
in which R¹, A¹, Z¹, L¹, L², and L³ have the meanings indicated in relation to the formula (II) and R² has the meanings indicated in relation to the formula (I).

9. Chromane derivatives of the general formulae (IIb1) to (IIb3) according to claim 8, characterised in that they have the following structures: 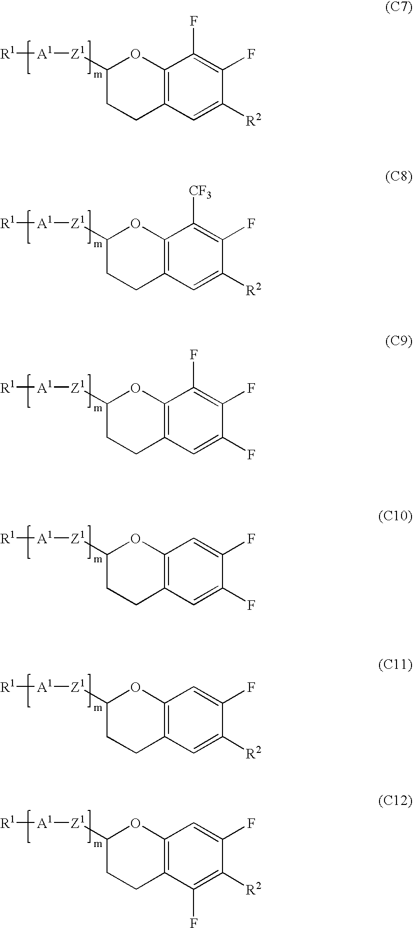
in which R¹, A¹ and Z¹ adopt the meanings indicated in relation to the formula (II), R² adopts the meanings indicated in relation to the formula (I), m=1, 2 or 3.

10. Chromane derivatives of the formula (II) according claim 5, characterised in that the compounds of the formula (II) are compounds having one ring in the mesogenic group R¹(-A¹-Z¹)ₘ- of the sub-formulae a and b
R¹-A²- a
R¹-A²-Z²- b.

11. Chromane derivatives of the formula (II) according to claim 5, characterised in that the compounds of the formula (II) are compounds having two rings in the mesogenic group R¹(-A¹-Z¹)ₘ- of the sub-formulae c to f
R¹-A¹-A²- c
R¹-A¹-A²-Z²- d
R¹-A¹-Z¹-A²- e
R¹-A¹-Z¹-A²-Z²- f.

12. Chromane derivatives of the formula (II) according to claim 5, characterised in that the compounds of the formula (II) are compounds having three rings in the mesogenic group R¹(-A¹-Z¹)ₘ- of the sub-formulae g to o
R¹-A¹-A¹-A²- g
R¹-A¹-Z¹-A¹-A²- h
R¹-A¹-A¹-Z¹-A²- i
R¹-A¹-A¹-A²-Z²- j
R¹-A¹-Z¹-A¹-Z¹-A²- k
R¹-A¹-Z¹-A¹-A²-Z²- m
R¹-A¹-A¹-Z¹-A²-Z²- n
R¹-A¹-Z¹-A¹-Z¹-A²-Z² o.

13. Chromane derivatives according to claim 10, characterised in that the compounds of the su-formula a are compounds of the following sub-formulae:

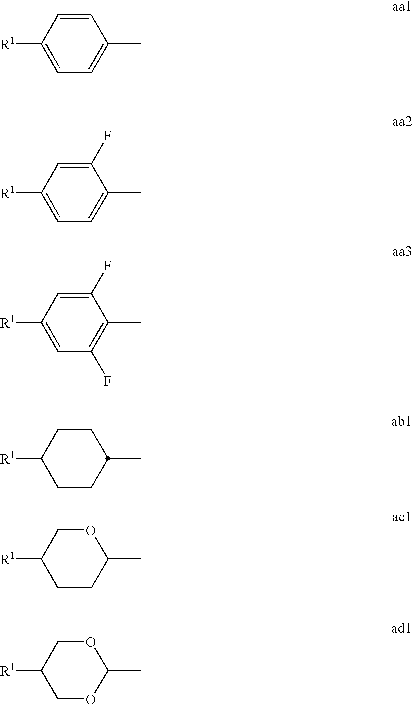

14. Chromane derivatives according to claim 11, characterised in that the compounds of the sub-formula c are compounds of the following sub-formulae:

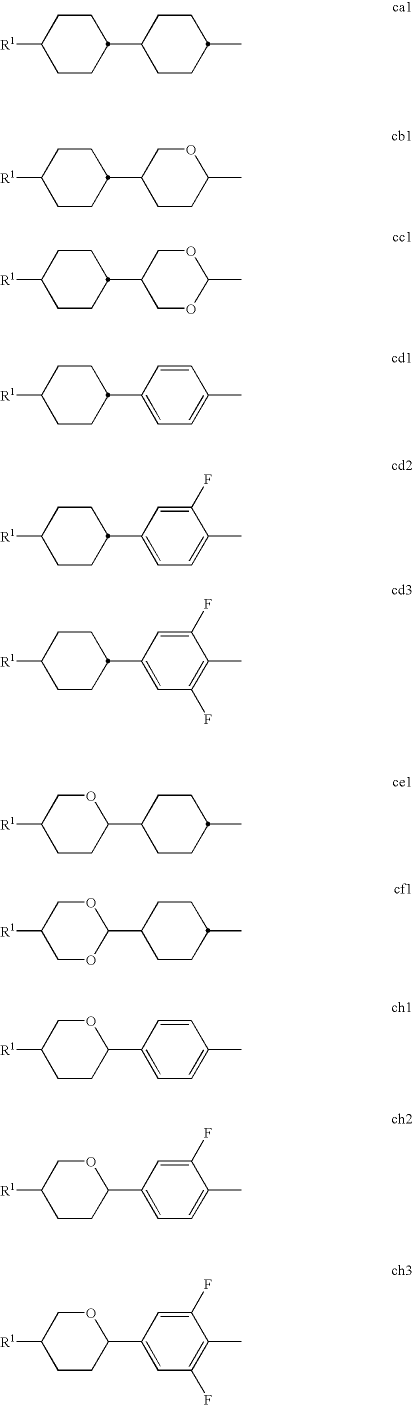

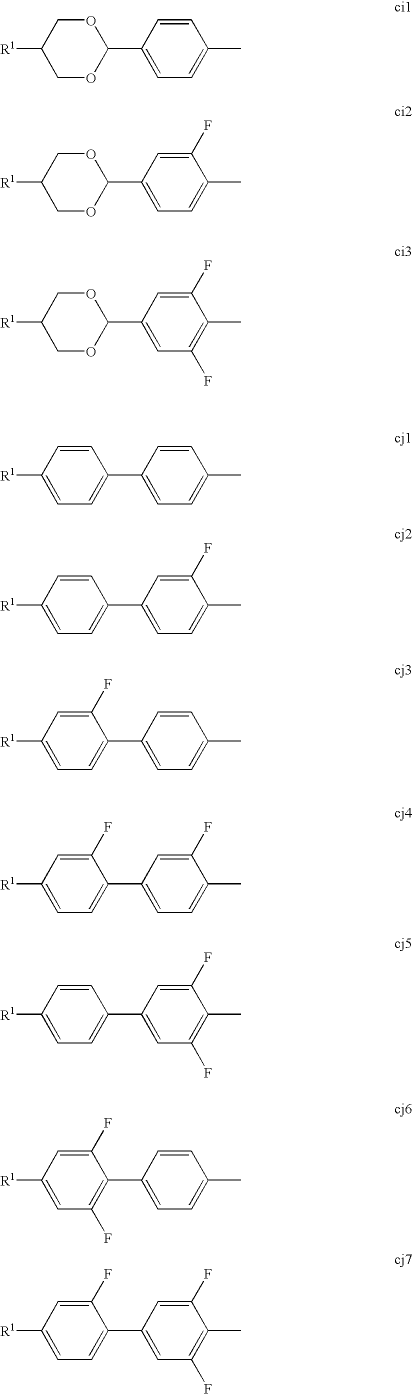

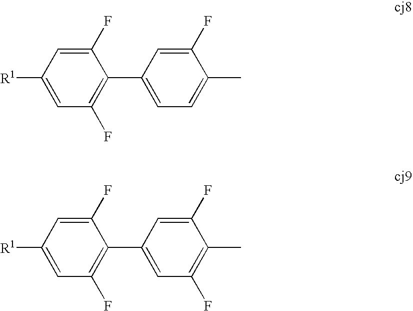

15. Chromane derivatives according to claim 11, characterised in that the compounds of the sub-formula d are compounds of the following sub-formulae:

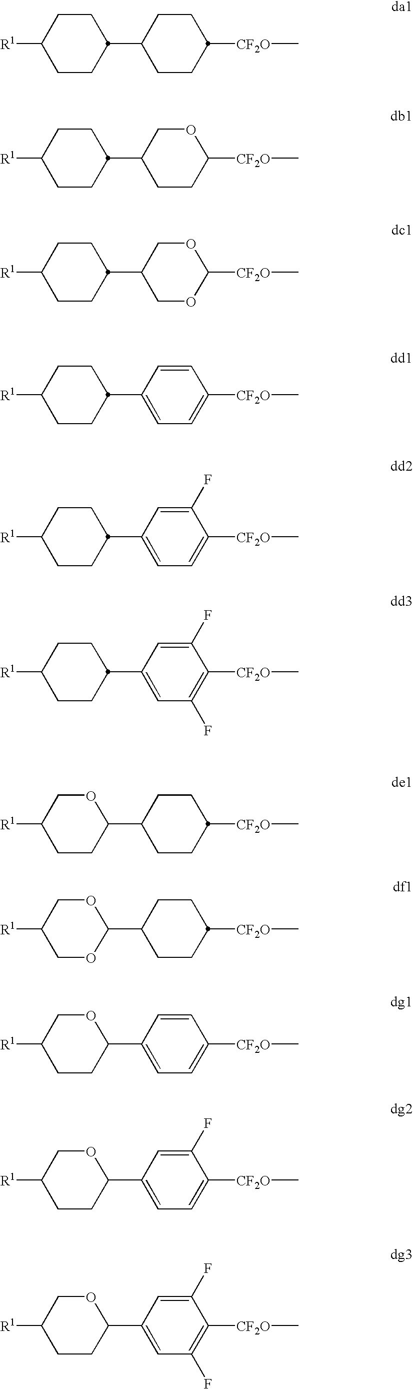

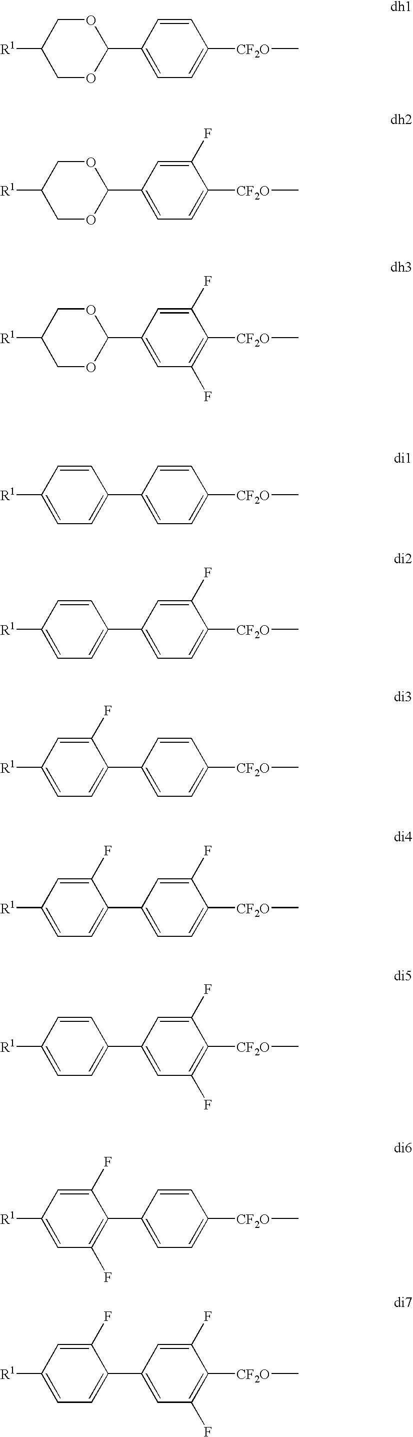

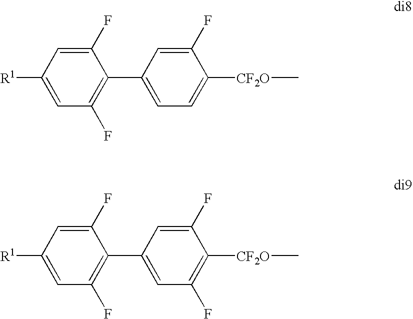

16. Chromane derivatives according to claim 11, characterised in that the compounds of the sub-formula e are compounds of the following sub-formulae:

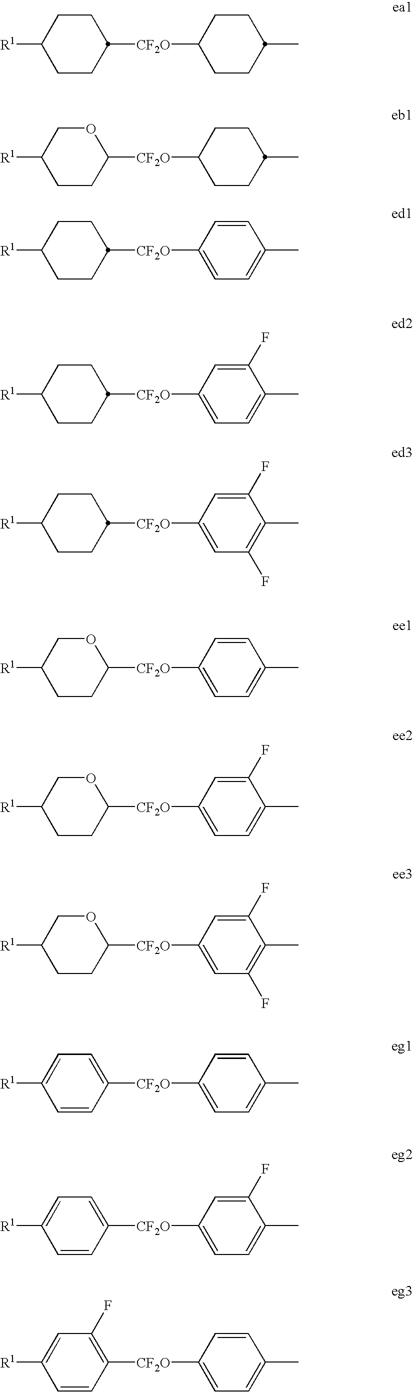

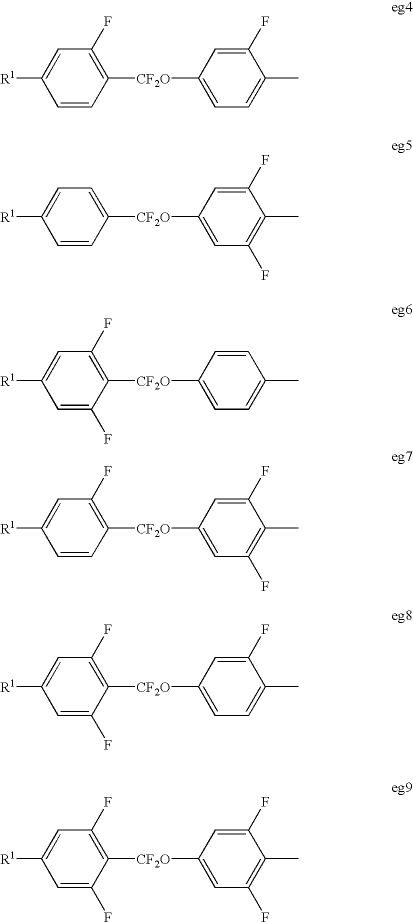

17. Use of chromane derivatives of the formulae (I) and (II) and chromene derivatives of the formulae (III) to (VI) according to claim 1 as component(s) in liquid-crystalline media.

18. Liquid-crystalline medium having at least two liquid-crystalline components characterised in that it comprises at least one chromane and/or chromene derivative of the formulae (I) to (VI) according to claim 1.

19. Liquid-crystal display element, characterised in that it contains a liquid-crystalline medium according to claim 18.

20. Electro-optical display element, characterised in that it contains, as dielectric, a liquid-crystalline medium according to claim 18.
